(19) United States
(12) Patent Application Publication
(10) Pub. No.: US 2018/0243225 A1
(43) Pub. Date: Aug. 30, 2018

(54) EBOLA/MARBURG VACCINES

(71) Applicant: ModernaTX, Inc., Cambridge, MA (US)

(72) Inventor: Giuseppe Ciaramella, Sudbury, MA (US)

(73) Assignee: ModernaTX, Inc., Cambridge, MA (US)

(21) Appl. No.: 15/880,436

(22) Filed: Jan. 25, 2018

Related U.S. Application Data
(60) Provisional application No. 62/450,537, filed on Jan. 25, 2017.

(51) Int. Cl. A61K 9/51; A61K 39/12; C12N 7/00; C07K 14/005; A61K 39/39; A61P 31/14

(52) U.S. Cl. CPC A61K 9/5123; A61K 39/12; C12N 7/00; A61K 2039/53; A61K 39/39; A61P 31/14; C07K 14/005

(57) ABSTRACT

The disclosure relates to Ebola virus and/or Marburg virus ribonucleic acid (RNA) vaccines, as well as methods of using the vaccines and compositions comprising the vaccines. The vaccines include one or more RNA polynucleotides having an open reading frame encoding Ebola virus and/or Marburg virus. Methods for preparing and using such vaccines are also described.

### RELATED APPLICATION

This application claims the benefit under 35 U.S.C. § 119(e) of U.S. provisional application No. 62/450,537, filed Jan. 25, 2017, which is incorporated by reference herein in its entirety.

### BACKGROUND

Ebola virus belongs to the Filoviridae family, similar to the Marburg virus. Filoviruses are relatively simple viruses of 19 Kb genomes and consist of seven genes which encode nucleoprotein (NP), glycoprotein (GP), four smaller viral proteins (VP24, VP30, VP35 and VP40), and the RNA-dependent RNA polymerase (L protein) all in a single strand of negative-sensed RNA. In general, minus-strand (−) RNA viruses, such as Ebola virus and Marburg virus, are major causes of human suffering that cause epidemics of serious human illness. In humans the diseases caused by these viruses include Ebola (Orthomyxoviridae), Marburg virus disease (Marburgvirus), mumps, measles, upper and lower respiratory tract disease (Paramyxoviridae), rabies (Rhabdoviridae), hemorrhagic fever (Filoviridae, Bunyaviridae and Arenaviridae), encephalitis (Bunyaviridae) and neurological illness (Bomaviridae). Due to the severity of disease caused by filoviruses, these viruses are considered a significant world health threat. For instance they have many of the characteristics commonly associated with biological weapons since they can be grown in large quantities, can be fairly stable, are highly infectious as an aerosol, and are exceptionally deadly.

Deoxyribonucleic acid (DNA) vaccination is one technique used to stimulate humoral and cellular immune responses to foreign antigens. The direct injection of genetically engineered DNA (e.g., naked plasmid DNA) into a living host results in a small number of its cells directly producing an antigen, resulting in a protective immunological response. With this technique, however, comes potential problems, including the possibility of insertional mutagenesis, which could lead to the activation of oncogenes or the inhibition of tumor suppressor genes.

### SUMMARY

Provided herein is a ribonucleic acid (RNA) vaccine that builds on the knowledge that RNA (e.g., messenger RNA (mRNA)) can safely direct the body's cellular machinery to produce nearly any protein of interest, from native proteins to antibodies and other entirely novel protein constructs that can have therapeutic activity inside and outside of cells. The RNA vaccines of the present disclosure may be used to induce a balanced immune response against Ebola virus and/or Marburg virus, comprising both cellular and humoral immunity, without risking the possibility of insertional mutagenesis, for example.

The RNA (e.g., mRNA) vaccines may be utilized in various settings depending on the prevalence of the infection or the degree or level of unmet medical need. The RNA (e.g., mRNA) vaccines may be utilized to treat and/or prevent an Ebola virus, a Marburg virus, or a combination of both viruses, of various genotypes, strains, and isolates. The RNA (e.g., mRNA) vaccines have superior properties in that they produce much larger antibody titers and produce responses earlier than commercially available anti-viral therapeutic treatments. As demonstrated in the examples, the mRNA vaccines described herein were capable of providing 100% protection against the Ebola viral infection in an animal model. While not wishing to be bound by theory, it is believed that the RNA vaccines, as mRNA polynucleotides, are better designed to produce the appropriate protein conformation upon translation as the RNA vaccines co-opt natural cellular machinery. Unlike traditional vaccines which are manufactured ex vivo and may trigger unwanted cellular responses, the RNA vaccines are presented to the cellular system in a more native fashion.

Some embodiments of the present disclosure provide Ebola virus (Ebola, EBOV) vaccines that include at least one RNA (e.g., mRNA) polynucleotide having an open reading frame encoding at least one Ebola antigenic polypeptide or an immunogenic fragment thereof (e.g., an immunogenic fragment capable of inducing an immune response to Ebola).

In some embodiments, the antigenic polypeptide is selected from EBOV glycoprotein (GP), surface EBOV GP, wild type EBOV GP, mature EBOV GP, secreted wild type EBOV GP, secreted mature EBOV GP, sGP, delta peptide (Δ-peptide), GP1, GP1,2Δ, or immunogenic fragments thereof or combinations thereof. In other embodiments the antigenic polypeptide is EBOV nucleoprotein NP, viral polymerase L, the polymerase cofactor VP35, the transcriptional activator VP30, VP24, or the matrix protein VP40

In some embodiments, the at least one antigenic polypeptide is from Ebola virus strain subtype Zaire, strain H. sapiens-wt/GIN/2014/Kissidougou-C15; subtype Bundibugyo, strain Uganda 2007; subtype Zaire, strain Mayinga 1976; subtype Sudan, strain Gulu, or a combination thereof.

Some embodiments of the present disclosure provide Marburg virus (Marburg, MARV) vaccines that include at least one RNA (e.g., mRNA) polynucleotide having an open reading frame encoding at least one Marburg antigenic polypeptide or an immunogenic fragment thereof (e.g., an immunogenic fragment capable of inducing an immune response to Marburg).

In some embodiments, the antigenic polypeptide is selected from MARV glycoprotein (GP), surface MARV GP, wild type MARV GP, mature MARV GP, secreted wild type MARV GP, secreted mature MARV GP, or combinations thereof.

In some aspects the invention is an Ebola/Marburg virus vaccine, comprising at least one RNA polynucleotide having an open reading frame encoding at least one Ebola virus or Marburg virus antigenic polypeptide, formulated in a cationic lipid nanoparticle.

In some embodiments, a RNA (e.g., mRNA) vaccine comprises at least one RNA (e.g., mRNA) polynucleotide having an open reading frame encoding at least one EBOV or MARV antigenic polypeptide. In some embodiments, at least one antigenic polypeptide is an EBOV or MARV polyprotein. In some embodiments, at least one antigenic polypeptide is major surface glycoprotein G or an immunogenic fragment thereof. In some embodiments, at least one antigenic polypeptide is sGP, delta peptide (Δ-peptide), GP1, GP1,2Δ, or immunogenic fragments thereof. The predominant products of the GP gene, sGP and delta peptide (Δ-peptide), are generated through furin cleavage from a precursor (pre-sGP) that is produced from nonedited mRNA species and are efficiently released from infected cells. In other embodiments the antigenic polypeptide is EBOV nucleoprotein NP, viral polymerase L, the polymerase cofactor VP35, the transcriptional activator VP30, VP24, or the matrix protein VP40.

In some embodiments, at least one EBOV antigenic polypeptide comprises an amino acid sequence of Tables 3, 5, or 9. In some embodiments, the amino acid sequence of the EBOV antigenic polypeptide is, or is a fragment of, or is a homolog or variant having at least 80% (e.g., 85%, 90%, 95%, 98%, 99%) identity to, the amino acid sequence of Tables 3, 5, or 9.

In some embodiments, at least one EBOV antigenic polypeptide is encoded by a nucleic acid sequence of Tables 3 or 4.

In some embodiments, at least one EBOV RNA (e.g., mRNA) polynucleotide is encoded by a nucleic acid sequence, or a fragment of or is a nucleotide sequence of Tables 6 or 8.

In some embodiments, at least one MARV antigenic polypeptide comprises an amino acid sequence of Table 10. In some embodiments, the amino acid sequence of the MARV antigenic polypeptide is, or is a fragment of, or is a homolog or variant having at least 80% (e.g., 85%, 90%, 95%, 98%, 99%) identity to, the amino acid sequence of Table 10.

In some embodiments, at least one MARV antigenic polypeptide is encoded by a nucleic acid sequence of Table 11.

In some embodiments, at least one MARV RNA (e.g., mRNA) polynucleotide is encoded by a nucleic acid sequence, or a fragment of or is a nucleotide sequence of Table 12.

In some embodiments, an open reading frame of a RNA (e.g., mRNA) vaccine is codon-optimized. In some embodiments, at least one RNA polynucleotide encodes at least one antigenic polypeptide having an amino acid sequence of Tables 6, 8, or 12 (see also amino acid sequences of Table 9) and is codon optimized mRNA.

In some embodiments, a RNA (e.g., mRNA) vaccine further comprises an adjuvant.

Table 9 provides National Center for Biotechnology Information (NCBI) accession numbers of interest. It should be understood that the phrase “an amino acid sequence of Table 9” refers to an amino acid sequence identified by one or more NCBI accession numbers listed in Table 9. Each of the amino acid sequences, and variants having greater than 95% identity or greater than 98% identity to each of the amino acid sequences encompassed by the accession numbers of Table 9 are included within the constructs (polynucleotides/polypeptides) of the present disclosure.

In some embodiments, at least one mRNA polynucleotide is encoded by a nucleic acid of Tables 6, 8, and 12 (see also Table 9) and having less than 80% identity to wild-type mRNA sequence. In some embodiments, at least one mRNA polynucleotide is encoded by a nucleic acid having a sequence of Tables 6, 8, and 12 (see also nucleic acid sequences of Table 9) and having less than 75%, 85% or 95% identity to a wild-type mRNA sequence. In some embodiments, at least one mRNA polynucleotide is encoded by a nucleic acid having a sequence of Tables 6, 8, and 12 (see also nucleic acid sequences of Table 9) and having less than 50-80%, 60-80%, 40-80%, 30-80%, 70-80%, 75-80% or 78-80% identity to wild-type mRNA sequence. In some embodiments, at least one mRNA polynucleotide is encoded by a nucleic acid of Tables 6, 8, and 12 (see also nucleic acid sequences of Table 9) and having less than 40-85%, 50-85%, 60-85%, 30-85%, 70-85%, 75-85% or 80-85% identity to wild-type mRNA sequence. In some embodiments, at least one mRNA polynucleotide is encoded by a nucleic acid having a sequence of Tables 6, 8, and 12 (see also nucleic acid sequences of Table 9) and having less than 40-90%, 50-90%, 60-90%, 30-90%, 70-90%, 75-90%, 80-90%, or 85-90% identity to wild-type mRNA sequence.

In some embodiments, at least one RNA polynucleotide encodes at least one antigenic polypeptide having an amino acid sequence of Tables 5 and 10 (see also amino acid sequences of Table 9) and having at least 80% (e.g., 85%, 90%, 95%, 98%, 99%) identity to wild-type mRNA sequence, but does not include wild-type mRNA sequence.

In some embodiments, at least one RNA polynucleotide encodes at least one antigenic polypeptide having an amino acid sequence of Tables 5 and 10; see also amino acid sequences of Table 9) and has less than 95%, 90%, 85%, 80% or 75% identity to wild-type mRNA sequence. In some embodiments, at least one RNA polynucleotide encodes at least one antigenic polypeptide having an amino acid sequence of Tables 5 and 10 (; see also amino acid sequences of Table 9) and has 30-80%, 40-80%, 50-80%, 60-80%, 70-80%, 75-80% or 78-80%, 30-85%, 40-85%, 50-805%, 60-85%, 70-85%, 75-85% or 78-85%, 30-90%, 40-90%, 50-90%, 60-90%, 70-90%, 75-90%, 80-90% or 85-90% identity to wild-type mRNA sequence.

In some embodiments, at least one RNA polynucleotide encodes at least one antigenic polypeptide having at least 90%, at least 95%, at least 96%, at least 97%, at least 98%, or at least 99% identity to an amino acid sequence of Tables 5 and 10 (see also amino acid sequences of Table 9). In some embodiments, at least one RNA polynucleotide encodes at least one antigenic polypeptide having 95%-99% identity to an amino acid sequence of Tables 5 and 10 (see also amino acid sequences of Table 9).

In some embodiments, at least one RNA polynucleotide encodes at least one antigenic polypeptide having at least 90%, at least 95%, at least 96%, at least 97%, at least 98%, or at least 99% identity to an amino acid sequence of Tables 5 and 10 (see also amino acid sequences of Table 9) and having membrane fusion activity. In some embodiments, at least one RNA polynucleotide encodes at least one antigenic polypeptide having 95%-99% identity to an amino acid sequence of Tables 5 and 10 (see also amino acid sequences of Table 9) and having membrane fusion activity.

In some embodiments, at least one RNA polynucleotide encodes at least one antigenic polypeptide (e.g., at least one EBOV antigenic polypeptide, at least one MARV antigenic polypeptide, or a combination of the foregoing antigenic polypeptides) that attaches to cell receptors.

In some embodiments, at least one RNA polynucleotide encodes at least one antigenic polypeptide (e.g., at least one EBOV antigenic polypeptide, at least one MARV antigenic polypeptide or a combination of the foregoing antigenic polypeptides) that causes fusion of viral and cellular membranes.

In some embodiments, at least one RNA polynucleotide encodes at least one antigenic polypeptide (e.g., at least one EBOV antigenic polypeptide, at least one MARV antigenic polypeptide, or a combination of the foregoing antigenic polypeptides) that is responsible for binding of the virus to a cell being infected.

Some embodiments of the present disclosure provide a vaccine that includes at least one ribonucleic acid (RNA) (e.g., mRNA) polynucleotide having an open reading frame encoding at least one antigenic polypeptide (e.g., at least one EBOV antigenic polypeptide, at least one MARV antigenic polypeptide, or a combination of the foregoing antigenic polypeptides), at least one 5′ terminal cap and at least one chemical modification, formulated within a lipid nanoparticle.

In some embodiments, a 5′ terminal cap is 7mG(5′)ppp(5′)NlmpNp.

In some embodiments, at least one chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, the chemical modification is in the 5-position of the uracil. In some embodiments, the chemical modification is a N1-methylpseudouridine. In some embodiments, the chemical modification is a N1-ethylpseudouridine.

In some embodiments, a lipid nanoparticle comprises a cationic lipid, a PEG-modified lipid, a sterol and a non-cationic lipid. In some embodiments, a cationic lipid is an ionizable cationic lipid and the non-cationic lipid is a neutral lipid, and the sterol is a cholesterol.

In some embodiments, a lipid nanoparticle comprises compounds of Formula (I) and/or Formula (II), discussed below.

In some embodiments, an Ebola/Marburg virus RNA (e.g., mRNA) vaccine is formulated in a lipid nanoparticle that comprises a compound selected from Compounds 3, 18, 20, 25, 26, 29, 30, 60, 108-112 and 122, described below.

Some embodiments of the present disclosure provide a vaccine that includes at least one RNA (e.g., mRNA) polynucleotide having an open reading frame encoding at least one antigenic polypeptide (e.g., at least one EBOV antigenic polypeptide, at least one MARV antigenic polypeptide, or a combination of the foregoing antigenic polypeptides), wherein at least 80% (e.g., 85%, 90%, 95%, 98%, 99%) of the uracil in the open reading frame have a chemical modification, optionally wherein the vaccine is formulated in a lipid nanoparticle (e.g., a lipid nanoparticle comprises a cationic lipid, a PEG-modified lipid, a sterol and a non-cationic lipid).

In some embodiments, 100% of the uracil in the open reading frame have a chemical modification. In some embodiments, a chemical modification is in the 5-position of the uracil. In some embodiments, a chemical modification is a N1-methyl pseudouridine. In some embodiments, 100% of the uracil in the open reading frame have a N1-methyl pseudouridine in the 5-position of the uracil.

In some embodiments, an open reading frame of a RNA (e.g., mRNA) polynucleotide encodes at least two antigenic polypeptides (e.g., at least two EBOV antigenic polypeptides, at least two MARV antigenic polypeptides, or a combination of the foregoing antigenic polypeptides). In some embodiments, the open reading frame encodes at least five or at least ten antigenic polypeptides. In some embodiments, the open reading frame encodes at least 100 antigenic polypeptides. In some embodiments, the open reading frame encodes 2-100 antigenic polypeptides.

In some embodiments, a vaccine comprises at least two RNA (e.g., mRNA) polynucleotides, each having an open reading frame encoding at least one antigenic polypeptide (e.g., at least one EBOV antigenic polypeptide, at least one MARV antigenic polypeptide, or a combination of the foregoing antigenic polypeptides). In some embodiments, the vaccine comprises at least five or at least ten RNA (e.g., mRNA) polynucleotides, each having an open reading frame encoding at least one antigenic polypeptide or an immunogenic fragment thereof. In some embodiments, the vaccine comprises at least 100 RNA (e.g., mRNA) polynucleotides, each having an open reading frame encoding at least one antigenic polypeptide. In some embodiments, the vaccine comprises 2-100 RNA (e.g., mRNA) polynucleotides, each having an open reading frame encoding at least one antigenic polypeptide.

In some embodiments, at least one antigenic polypeptide (e.g., at least one EBOV antigenic polypeptide, at least one MARV antigenic polypeptide, or a combination of the foregoing antigenic polypeptides) is fused to a signal peptide. In some embodiments, the signal peptide is selected from: a HuIgGk signal peptide (METPAQLLFLLLLWLPDTTG; SEQ ID NO: 178); IgE heavy chain epsilon-1 signal peptide (MDWTWILFLVAAATRVHS; SEQ ID NO: 179); Japanese encephalitis PRM signal sequence (MLGSNSGQRVVFTILLLLVAPAYS; SEQ ID NO: 180), VSVg protein signal sequence (MKCLLYLAFLFIGVNCA; SEQ ID NO: 181) and Japanese encephalitis JEV signal sequence (MWLVSLAIVTACAGA; SEQ ID NO: 182).

In some embodiments, the signal peptide is fused to the N-terminus of at least one antigenic polypeptide. In some embodiments, a signal peptide is fused to the C-terminus of at least one antigenic polypeptide.

In some embodiments, at least one antigenic polypeptide (e.g., at least one EBOV antigenic polypeptide, at least one MARV antigenic polypeptide, or a combination of the foregoing antigenic polypeptides) comprises a mutated N-linked glycosylation site.

Also provided herein is a RNA (e.g., mRNA) vaccine of any one of the foregoing paragraphs (e.g., an EBOV vaccine, a MARV vaccine, or a combination of the foregoing vaccines), formulated in a nanoparticle (e.g., a lipid nanoparticle).

In some embodiments, the nanoparticle has a mean diameter of 50-200 nm. In some embodiments, the nanoparticle is a lipid nanoparticle. In some embodiments, the lipid nanoparticle comprises a cationic lipid, a PEG-modified lipid, a sterol and a non-cationic lipid. In some embodiments, the lipid nanoparticle comprises a molar ratio of about 20-60% cationic lipid, 0.5-15% PEG-modified lipid, 25-55% sterol, and 25% non-cationic lipid. In some embodiments, the cationic lipid is an ionizable cationic lipid and the non-cationic lipid is a neutral lipid, and the sterol is a cholesterol. In some embodiments, the cationic lipid is selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

In some embodiments, a lipid nanoparticle comprises compounds of Formula (I) and/or Formula (II), as discussed below.

In some embodiments, a lipid nanoparticle comprises Compounds 3, 18, 20, 25, 26, 29, 30, 60, 108-112, or 122, as discussed below.

In some embodiments, the nanoparticle has a polydispersity value of less than 0.4 (e.g., less than 0.3, 0.2 or 0.1).

In some embodiments, the nanoparticle has a net neutral charge at a neutral pH value.

In some embodiments, the virus vaccine is multivalent.

Some embodiments of the present disclosure provide methods of inducing an antigen specific immune response in a subject, comprising administering to the subject any of the RNA (e.g., mRNA) vaccine as provided herein in an amount effective to produce an antigen-specific immune response. In some embodiments, the RNA (e.g., mRNA) vaccine is a EBOV vaccine or a MARV vaccine. In some embodiments, the RNA (e.g., mRNA) vaccine is a combination vaccine comprising a combination of the foregoing vaccines.

In some embodiments, an antigen-specific immune response comprises a T cell response or a B cell response.

In some embodiments, a method of producing an antigen-specific immune response comprises administering to a subject a single dose (no booster dose) of a RNA (e.g., mRNA) vaccine of the present disclosure. In some embodiments, the RNA (e.g., mRNA) vaccine is a EBOV vaccine or a MARV vaccine. In some embodiments, the RNA (e.g., mRNA) vaccine is a combination vaccine comprising a combination of any two or more of the foregoing vaccines.

In some embodiments, a method further comprises administering to the subject a second (booster) dose of a RNA (e.g., mRNA) vaccine. Additional doses of a RNA (e.g., mRNA) vaccine may be administered.

In some embodiments, the subjects exhibit a seroconversion rate of at least 80% (e.g., at least 85%, at least 90%, or at least 95%) following the first dose or the second (booster) dose of the vaccine. Seroconversion is the time period during which a specific antibody develops and becomes detectable in the blood. After seroconversion has occurred, a virus can be detected in blood tests for the antibody. During an infection or immunization, antigens enter the blood, and the immune system begins to produce antibodies in response. Before seroconversion, the antigen itself may or may not be detectable, but antibodies are considered absent. During seroconversion, antibodies are present but not yet detectable. Any time after seroconversion, the antibodies can be detected in the blood, indicating a prior or current infection.

In some embodiments, a RNA (e.g., mRNA) vaccine is administered to a subject by intradermal or intramuscular injection.

Some embodiments, of the present disclosure provide methods of inducing an antigen specific immune response in a subject, including administering to a subject a RNA (e.g., mRNA) vaccine in an effective amount to produce an antigen specific immune response in a subject. Antigen-specific immune responses in a subject may be determined, in some embodiments, by assaying for antibody titer (for titer of an antibody that binds to an EBOV and/or a MARV antigenic polypeptide) following administration to the subject of any of the RNA (e.g., mRNA) vaccines of the present disclosure. In some embodiments, the anti-antigenic polypeptide antibody titer produced in the subject is increased by at least 1 log relative to a control. In some embodiments, the anti-antigenic polypeptide antibody titer produced in the subject is increased by 1-3 log relative to a control.

In some embodiments, the anti-antigenic polypeptide antibody titer produced in a subject is increased at least 2 times relative to a control. In some embodiments, the anti-antigenic polypeptide antibody titer produced in the subject is increased at least 5 times relative to a control. In some embodiments, the anti-antigenic polypeptide antibody titer produced in the subject is increased at least 10 times relative to a control. In some embodiments, the anti-antigenic polypeptide antibody titer produced in the subject is increased 2-10 times relative to a control.

In some embodiments, the control is an anti-antigenic polypeptide antibody titer produced in a subject who has not been administered a RNA (e.g., mRNA) vaccine of the present disclosure. In some embodiments, the control is an anti-antigenic polypeptide antibody titer produced in a subject who has been administered a live attenuated or inactivated EBOV and/or MARV vaccine and/or BetaCoV vaccine (see, e.g., Ren J. et al. J of Gen. Virol. 2015; 96: 1515-1520), or wherein the control is an anti-antigenic polypeptide antibody titer produced in a subject who has been administered a recombinant or purified EBOV and/or MARV protein vaccine. In some embodiments, the control is an anti-antigenic polypeptide antibody titer produced in a subject who has been administered an EBOV and/or MARV virus-like particle (VLP) vaccine (see, e.g., Cox R G et al., J Virol. 2014 June; 88(11): 6368-6379).

A RNA (e.g., mRNA) vaccine of the present disclosure is administered to a subject in an effective amount (an amount effective to induce an immune response). In some embodiments, the effective amount is a dose equivalent to an at least 2-fold, at least 4-fold, at least 10-fold, at least 100-fold, at least 1000-fold reduction in the standard of care dose of a recombinant EBOV and/or MARV protein vaccine, wherein the anti-antigenic polypeptide antibody titer produced in the subject is equivalent to an anti-antigenic polypeptide antibody titer produced in a control subject administered the standard of care dose of a recombinant EBOV and/or MARV protein vaccine, a purified EBOV and/or MARV protein vaccine, a live attenuated EBOV and/or MARV vaccine, an inactivated EBOV and/or MARV vaccine, or a EBOV and/or MARV VLP vaccine. In some embodiments, the effective amount is a dose equivalent to 2-1000-fold reduction in the standard of care dose of a recombinant EBOV and/or MARV protein vaccine, wherein the anti-antigenic polypeptide antibody titer produced in the subject is equivalent to an anti-antigenic polypeptide antibody titer produced in a control subject administered the standard of care dose of a recombinant EBOV and/or MARV protein vaccine, a purified EBOV and/or MARV protein vaccine, a live attenuated EBOV and/or MARV vaccine, an inactivated EBOV and/or MARV vaccine, or a EBOV and/or MARV VLP vaccine.

In some embodiments, the control is an anti-antigenic polypeptide antibody titer produced in a subject who has been administered a virus-like particle (VLP) vaccine comprising structural proteins of EBOV and/or MARV.

A nucleic acid vaccine having one or more RNA polynucleotides having an open reading frame encoding an Ebola antigen and/or Marburg virus and a pharmaceutically acceptable carrier or excipient are provided in aspects of the invention. In some embodiments the Ebola antigen is an Ebola virus (EBOV) glycoprotein (GP). In other embodiments the Ebola antigen is a surface GP. In yet other embodiments the Ebola antigen is a wild type EBOV pro-GP, a wild type EBOV pro-GP-V5, a mature EBOV GP, a mature EBOV GP-V5, a secreted wild type EBOV pro-GP, a secreted wild type EBOV pro-GP-V5, a secreted mature EBOV GP, or a secreted mature EBOV GP-V5. In some embodiments the Marburg antigen is a Marburg virus (MARV) glycoprotein (GP). In other embodiments the Marburg antigen is a surface GP.

In some embodiments, the efficacy (or effectiveness) of a RNA (e.g., mRNA) vaccine is greater than 60%. In some embodiments, the RNA (e.g., mRNA) polynucleotide of the vaccine at least one EBOV antigenic polypeptide, at least one MARV antigenic polypeptide, or a combination of the foregoing antigenic polypeptides.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

Efficacy=(ARU−ARV)/ARU×100; and

Efficacy=(1−RR)×100.

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

Effectiveness=(1−OR)×100.

In some embodiments, the efficacy (or effectiveness) of a RNA (e.g., mRNA) vaccine is at least 65%, at least 70%, at least 75%, at least 80%, at least 85%, or at least 90%.

In some embodiments, the vaccine immunizes the subject against EBOV, MARV, or a combination of the foregoing viruses for up to 2 years. In some embodiments, the vaccine immunizes the subject against EBOV, MARV, or a combination of the foregoing viruses for more than 2 years, more than 3 years, more than 4 years, or for 5-10 years.

In some embodiments, the subject is about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger.

In some embodiments, the subject was born full term (e.g., about 37-42 weeks). In some embodiments, the subject was born prematurely, for example, at about 36 weeks of gestation or earlier (e.g., about 36, 35, 34, 33, 32, 31, 30, 29, 28, 27, 26 or 25 weeks). For example, the subject may have been born at about 32 weeks of gestation or earlier. In some embodiments, the subject was born prematurely between about 32 weeks and about 36 weeks of gestation. In such subjects, a RNA (e.g., mRNA) vaccine may be administered later in life, for example, at the age of about 6 months to about 5 years, or older.

In some embodiments, the subject is pregnant (e.g., in the first, second or third trimester) when administered an RNA (e.g., mRNA) vaccine. Thus, the present disclosure provides RNA (e.g., mRNA) vaccines for maternal immunization to improve mother-to-child transmission of protection against the virus.

In some embodiments, the subject is a young adult between the ages of about 20 years and about 50 years (e.g., about 20, 25, 30, 35, 40, 45 or 50 years old).

In some embodiments, the subject is an elderly subject about 60 years old, about 70 years old, or older (e.g., about 60, 65, 70, 75, 80, 85 or 90 years old).

In some embodiments, the subject is has a chronic pulmonary disease (e.g., chronic obstructive pulmonary disease (COPD) or asthma). Two forms of COPD include chronic bronchitis, which involves a long-term cough with mucus, and emphysema, which involves damage to the lungs over time. Thus, a subject administered a RNA (e.g., mRNA) vaccine may have chronic bronchitis or emphysema.

In some embodiments, the subject has been exposed to EBOV, MARV, or both viruses; the subject is infected with EBOV, MARV, or both viruses; or subject is at risk of infection by EBOV, MARV, or both viruses.

In some embodiments, the subject is immunocompromised (has an impaired immune system, e.g., has an immune disorder or autoimmune disorder).

In some embodiments the nucleic acid vaccines described herein are chemically modified. In other embodiments the nucleic acid vaccines are unmodified.

Yet other aspects provide compositions for and methods of vaccinating a subject comprising administering to the subject a nucleic acid vaccine comprising one or more RNA polynucleotides having an open reading frame encoding a first virus antigenic polypeptide, wherein the RNA polynucleotide does not include a stabilization element, and wherein an adjuvant is not coformulated or co-administered with the vaccine.

In some embodiments, the nucleic acid vaccine is administered to the subject by intradermal or intramuscular injection. In some embodiments, the nucleic acid vaccine is administered to the subject on day zero. In some embodiments, a second dose of the nucleic acid vaccine is administered to the subject on day twenty one.

In some embodiments, a dosage of 25 micrograms of the RNA polynucleotide is included in the nucleic acid vaccine administered to the subject. In some embodiments, a dosage of 100 micrograms of the RNA polynucleotide is included in the nucleic acid vaccine administered to the subject. In some embodiments, a dosage of 50 micrograms of the RNA polynucleotide is included in the nucleic acid vaccine administered to the subject. In some embodiments, a dosage of 75 micrograms of the RNA polynucleotide is included in the nucleic acid vaccine administered to the subject. In some embodiments, a dosage of 150 micrograms of the RNA polynucleotide is included in the nucleic acid vaccine administered to the subject. In some embodiments, a dosage of 400 micrograms of the RNA polynucleotide is included in the nucleic acid vaccine administered to the subject. In some embodiments, a dosage of 200 micrograms of the RNA polynucleotide is included in the nucleic acid vaccine administered to the subject. In some embodiments, the RNA polynucleotide accumulates at a 100 fold higher level in the local lymph node in comparison with the distal lymph node. In other embodiments the nucleic acid vaccine is chemically modified and in other embodiments the nucleic acid vaccine is not chemically modified.

Aspects of the invention provide a nucleic acid vaccine comprising one or more RNA polynucleotides having an open reading frame encoding a first antigenic polypeptide, wherein the RNA polynucleotide does not include a stabilization element, and a pharmaceutically acceptable carrier or excipient, wherein an adjuvant is not included in the vaccine. In some embodiments, the stabilization element is a histone stem-loop. In some embodiments, the stabilization element is a nucleic acid sequence having increased GC content relative to wild type sequence.

Aspects provide nucleic acid vaccines comprising one or more RNA polynucleotides having an open reading frame comprising at least one chemical modification or optionally no nucleotide modification, the open reading frame encoding a first antigenic polypeptide, wherein the RNA polynucleotide is present in the formulation for in vivo administration to a host such that the level of antigen expression in the host significantly exceeds a level of antigen expression produced by an mRNA vaccine having a stabilizing element or formulated with an adjuvant and encoding the first antigenic polypeptide.

Other aspects provide nucleic acid vaccines comprising one or more RNA polynucleotides having an open reading frame comprising at least one chemical modification or optionally no nucleotide modification, the open reading frame encoding a first antigenic polypeptide, wherein the vaccine has at least 10 fold less RNA polynucleotide than is required for an unmodified mRNA vaccine to produce an equivalent antibody titer. In some embodiments, the RNA polynucleotide is present in a dosage of 25-100 micrograms.

Aspects of the invention provide methods of creating, maintaining or restoring antigenic memory to a virus strain in an individual or population of individuals comprising administering to said individual or population an antigenic memory booster nucleic acid vaccine comprising (a) at least one RNA polynucleotide, said polynucleotide comprising at least one chemical modification or optionally no nucleotide modification and two or more codon-optimized open reading frames, said open reading frames encoding a set of reference antigenic polypeptides, and (b) optionally a pharmaceutically acceptable carrier or excipient. In some embodiments, the vaccine is administered to the individual via a route selected from the group consisting of intramuscular administration, intradermal administration and subcutaneous administration. In some embodiments, the administering step comprises contacting a muscle tissue of the subject with a device suitable for injection of the composition. In some embodiments, the administering step comprises contacting a muscle tissue of the subject with a device suitable for injection of the composition in combination with electroporation.

Other aspects provide nucleic acid vaccines comprising an LNP formulated RNA polynucleotide having an open reading frame comprising no nucleotide modifications (unmodified), the open reading frame encoding a first antigenic polypeptide, wherein the vaccine has at least 10 fold less RNA polynucleotide than is required for an unmodified mRNA vaccine not formulated in a LNP to produce an equivalent antibody titer. In some embodiments, the RNA polynucleotide is present in a dosage of 25-100 micrograms.

In other aspects the invention encompasses a method of treating an elderly subject age 60 years or older comprising administering to the subject a nucleic acid vaccine comprising one or more RNA polynucleotides having an open reading frame encoding a virus antigenic polypeptide in an effective amount to vaccinate the subject.

In other aspects the invention encompasses a method of treating a young subject age 17 years or younger comprising administering to the subject a nucleic acid vaccine comprising one or more RNA polynucleotides having an open reading frame encoding a virus antigenic polypeptide in an effective amount to vaccinate the subject.

In other aspects the invention encompasses a method of treating an adult subject comprising administering to the subject a nucleic acid vaccine comprising one or more RNA polynucleotides having an open reading frame encoding a virus antigenic polypeptide in an effective amount to vaccinate the subject.

In some aspects the invention is a method of vaccinating a subject with a combination vaccine including at least two nucleic acid sequences encoding antigens wherein the dosage for the vaccine is a combined therapeutic dosage wherein the dosage of each individual nucleic acid encoding an antigen is a sub therapeutic dosage. In some embodiments, the combined dosage is 25 micrograms of the RNA polynucleotide in the nucleic acid vaccine administered to the subject. In some embodiments, the combined dosage is 100 micrograms of the RNA polynucleotide in the nucleic acid vaccine administered to the subject. In some embodiments the combined dosage is 50 micrograms of the RNA polynucleotide in the nucleic acid vaccine administered to the subject. In some embodiments, the combined dosage is 75 micrograms of the RNA polynucleotide in the nucleic acid vaccine administered to the subject. In some embodiments, the combined dosage is 150 micrograms of the RNA polynucleotide in the nucleic acid vaccine administered to the subject. In some embodiments, the combined dosage is 400 micrograms of the RNA polynucleotide in the nucleic acid vaccine administered to the subject. In some embodiments, the sub therapeutic dosage of each individual nucleic acid encoding an antigen is 1, 2, 3, 4, 5, 6, 7, 8, 9, 10, 11, 12, 13, 14, 15, 16, 17, 18, 19, or 20 micrograms or any range combination thereof. In other embodiments the nucleic acid vaccine is chemically modified and in other embodiments the nucleic acid vaccine is not chemically modified.

The RNA polynucleotide is from Tables 6, 8, or 12 and includes at least one chemical modification. In other embodiments the RNA polynucleotide is from Tables 6, 8, or 12 and does not include any nucleotide modifications, or is unmodified. In yet other embodiments the at least one RNA polynucleotide encodes an antigenic protein of Tables 5 or 10 and includes at least one chemical modification. In other embodiments the RNA polynucleotide encodes an antigenic protein of Tables 5 or 10 and does not include any nucleotide modifications, or is unmodified.

In preferred aspects, vaccines of the invention (e.g., LNP-encapsulated mRNA vaccines) produce prophylactically- and/or therapeutically-efficacious levels, concentrations and/or titers of antigen-specific antibodies in the blood or serum of a vaccinated subject. As defined herein, the term antibody titer refers to the amount of antigen-specific antibody produces in s subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA). In exemplary embodiments, antibody titer is determined or measured by neutralization assay, e.g., by microneutralization assay. In certain aspects, antibody titer measurement is expressed as a ratio, such as 1:40, 1:100, etc.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead being placed upon illustrating the principles of various embodiments of the invention.

FIG. 1 shows a schematic depiction of the structure of Ebola glycoprotein (GP) and antigen constructs.

FIG. 2 shows the study design for the immunogenicity evaluation.

FIG. 3 is a graph depicting the initial anti-Ebola GP response at Day 10 after a single primary challenge. The positive control indicates the OD of the standard curve from 10 U/ml to 1 U/ml of mouse anti-Ebola GP mAb.

FIG. 4 shows the anti-Ebola GP antibody titer of selected antigens on Day 21 and Day 23 (n=3 animals per group).

FIG. 5 shows the anti-Ebola GP response at Day 21 post-vaccination.

FIG. 6 shows the in vitro neutralization activity of serum samples in the DeltaVp30 Ebola virus system.

FIG. 7 shows a schematic of an Ebola vaccine study in a Guinea Pig model.

FIG. 8 is a set of graphs depicting data in terms of survival, weight, temperature and score from the vaccination study shown in FIG. 7. Vaccination conferred 100% protection against 10E3 PFUs of gp-adapted Ebola (Zaire species, Mayinga strain).

FIG. 9 is a graph showing the in vivo neutralization activity of different mRNA constructs from three strains: Uganda (group 8), Ravin (group 9), and Musoke (group 10).

### DETAILED DESCRIPTION

Embodiments of the present disclosure provide RNA (e.g., mRNA) vaccines that include polynucleotide encoding an Ebola virus and/or a Marburg virus antigen. Ebola virus and/or Marburg RNA vaccines, as provided herein may be used to induce a balanced immune response, comprising both cellular and humoral immunity, without many of the risks associated with DNA vaccination.

The invention involves, in some aspects, the surprising finding that lipid nanoparticle (LNP) formulations significantly enhance the effectiveness of mRNA vaccines, including chemically modified and unmodified mRNA vaccines.

In addition to providing an enhanced immune response, the formulations of the invention generate a more rapid immune response with fewer doses of antigen than other vaccines. The mRNA-LNP formulations of the invention also produce quantitatively and qualitatively better immune responses than vaccines formulated in different carriers.

It was discovered quite surprisingly, according to the invention that vaccination with the mRNA vaccine of the invention conferred 100% protection against 10E3 PFUs of gp-adapted Ebola (Zaire species, Mayinga strain) in a guinea pig model of Ebola virus infection. In contrast to the vaccines of the invention, untreated animals succumbed to the infection completely by day 10 post infection.

In some embodiments, the amino acid sequence of the Ebola antigen or fragment thereof comprises at least 90%, 91%, 92%, 93%, 94%, 95%, 96%, 97% 98%, or 99% identity with any of the amino acid sequences provided in Tables 3, 5 and 9. In other embodiments, the nucleic acid sequence of the mRNA encoding the Ebola antigen or fragment thereof comprises at least 90%, 91%, 92%, 93%, 94%, 95%, 96%, 97% 98%, or 99% identity with any of the nucleic acid sequences provided in Tables 3, 4 and 7. In other embodiments, the nucleic acid sequence of the mRNA encoding the Ebola antigen or fragment thereof is encoded by a nucleic acid sequence comprising at least 90%, 91%, 92%, 93%, 94%, 95%, 96%, 97% 98%, or 99% identity with any of the nucleic acid sequences provided in Tables 3, 4 and 7.

In some embodiments, the amino acid sequence of the Marburg antigen or fragment thereof comprises at least 90%, 91%, 92%, 93%, 94%, 95%, 96%, 97% 98%, or 99% identify with any of the amino acid sequences provided in Table 10. In other embodiments, the nucleic acid sequence of the mRNA encoding the Marburg antigen or fragment thereof comprises at least 90%, 91%, 92%, 93%, 94%, 95%, 96%, 97% 98%, or 99% identity with any of the nucleic acid sequences provided in Table 11. In other embodiments, the nucleic acid sequence of the mRNA encoding the Marburg antigen or fragment thereof is encoded by a nucleic acid sequence comprising at least 90%, 91%, 92%, 93%, 94%, 95%, 96%, 97% 98%, or 99% identity with any of the nucleic acid sequences provided in Table 11.

Thus, the RNA (e.g., mRNA) vaccines of the present invention comprise one or more polynucleotides, e.g., polynucleotide constructs, which encode one or more wild type or engineered antigens. Exemplary polynucleotides, e.g., polynucleotide constructs, include antigen-encoding mRNA polynucleotides. In an exemplary aspect, polynucleotides of the invention, e.g., antigen-encoding RNA polynucleotides, may include at least one chemical modification. In exemplary aspects, polynucleotides of the invention, e.g., antigen-encoding RNA polynucleotides, may be fully modified (e.g., chemically modified) with respect to one or more nucleobases.

In some embodiments, the RNA vaccine of the invention is a polynucleotide encoding an Ebola virus antigen. There are five Ebola viruses within the genus Ebolavirus. Four of the five known ebolaviruses cause a severe and often fatal hemorrhagic fever in humans and other mammals, known as Ebola virus disease (EVD). The Ebola glycoprotein (GP) is the only virally expressed protein on the virion surface, where it is essential for the attachment to host cells and catalyzes membrane fusion. As a result, the Ebola GP is a critical component of vaccines, as well as a target of neutralizing antibodies and inhibitors of attachment and fusion. Pre-GP is cleaved by furin at a multi-basic motif into two subunits, GP1 and GP2, which remain associated through a disulfide linkage between Cys53 of GP1 and Cys609 of GP2. The heterodimer (GP1 and GP2) then assembles into a 450-kDa trimer (3 GP1 and 3 GP2) at the surface of nascent virions, where it exerts its functions.

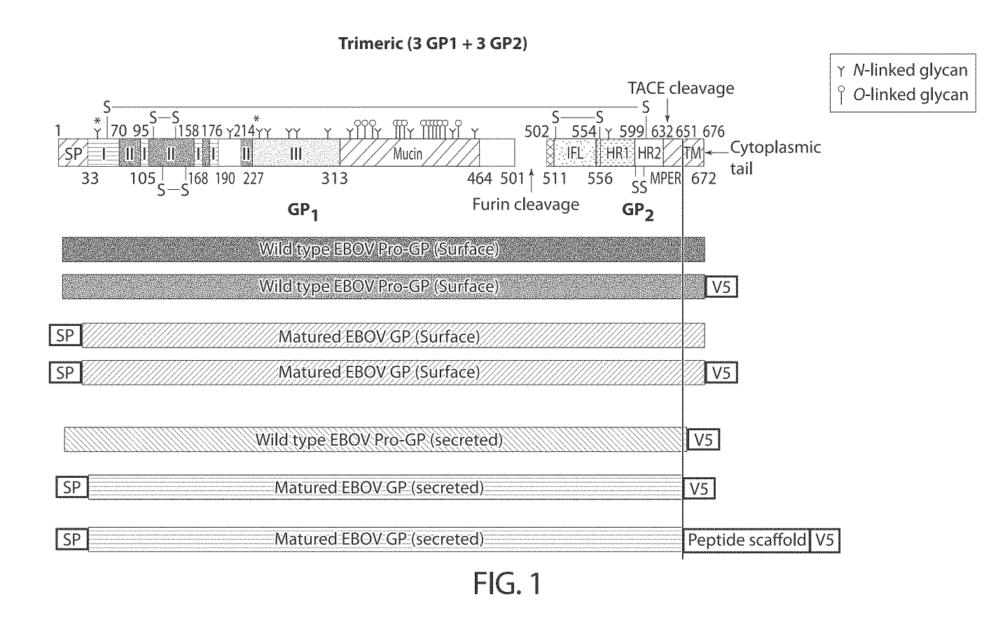

In some embodiments the Ebola antigen in the nucleic acid vaccine is an EBOV glycoprotein (GP). In other embodiments the Ebola antigen is a surface GP. Exemplary Ebola GP and antigen constructs tested herein are shown in FIG. 1 and Table 1.

The Ebola antigen may be a wild type EBOV pro-GP or a mature EBOV GP. “Mature” EBOV GP has been engineered to include a human signal peptide. Alternatively the Ebola antigen may be a secreted wild type EBOV pro-GP or mature EBOV GP. “Secreted” EBOV GP has been engineered to remove the transmembrane domain, i.e., residues 651-676. The constructs may also include V5.

Existing Ebola vaccines include conventional inactivated (by heat, formalin, or γ-irradiation) viral vaccines, sub-unit Ebola virus genes inserted into a DNA plasmid and Virus-like-particles (VLP) based on VP40 alone or with GP. Studies using conventional inactivated and DNA vaccines have shown consistently low survival rates in non-human primates. The mRNA Ebola vaccines of the invention have unique advantages over conventional vaccines. As shown in the data presented in the Examples the constructs of the invention provided 100% protection against infection in an animal model of Ebola.

The fourth gene of the Ebola genome encodes a 160-kDa envelope-attached glycoprotein (GP) and a 110 kDa secreted glycoprotein (sGP). Both GP and sGP have an identical 295-residue N-terminus, however, they have different C-terminal sequences. GP is a class I fusion protein which assembles as trimers on viral surface and plays an important role in virus entry and attachment. Mature GP is a disulfide-linked heterodimer formed by two subunits, GP1 and GP2, which are generated from the proteolytic process of GP precursor (pre-GP) by cellular furin during virus assembly. The GP1 subunit contains a mucin domain (Muc) and a receptor-binding domain (RBD); the GP2 subunit has a fusion peptide, a helical heptad-repeat (HR) region, a transmembrane (TM) domain, and a 4-residue cytoplasmic tail. The RBD of GP1 mediates the interaction of Ebola virus with cellular receptors such as DC-SIGN/LSIGN, TIM-1, hMGL, NPC1, β-integrins, folate receptor-α, and Tyro3 family receptors. The mucin domain has N- and O-linked glycans and enhances the viral attachment to cellular hMGL, and participates in shielding key neutralization epitopes, which helps the virus evade host immune responses.

The entire contents of International Application No. PCT/US2015/02740 is incorporated herein by reference.

### Nucleic Acids/Polynucleotides

Ebola virus and/or Marburg virus vaccines, as provided herein, comprise at least one (one or more) RNA (e.g., mRNA) polynucleotide having an open reading frame encoding at least one Ebola virus and/or Marburg virus antigenic polypeptide. The term “nucleic acid,” in its broadest sense, includes any compound and/or substance that comprises a polymer of nucleotides. These polymers are referred to as polynucleotides.

Nucleic acids (also referred to as polynucleotides) may be or may include, for example, RNAs, deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof.

In some embodiments, polynucleotides of the present disclosure function as messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo.

The basic components of an mRNA molecule typically include at least one coding region, a 5′ untranslated region (UTR), a 3′ UTR, a 5′ cap and a poly-A tail. Polynucleotides of the present disclosure may function as mRNA but can be distinguished from wild-type mRNA in their functional and/or structural design features which serve to overcome existing problems of effective polypeptide expression using nucleic-acid based therapeutics.

In some embodiments, a RNA polynucleotide of an Ebola virus and/or Marburg virus vaccine encodes 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9 or 9-10 antigenic polypeptides. In some embodiments, a RNA polynucleotide of an Ebola virus and/or Marburg virus vaccine encodes at least 10, 20, 30, 40, 50, 60, 70, 80, 90 or 100 antigenic polypeptides. In some embodiments, a RNA polynucleotide of an Ebola virus and/or Marburg virus vaccine encodes at least 100 or at least 200 antigenic polypeptides. In some embodiments, a RNA polynucleotide of an Ebola virus and/or Marburg vaccine encodes 1-10, 5-15, 10-20, 15-25, 20-30, 25-35, 30-40, 35-45, 40-50, 1-50, 1-100, 2-50 or 2-100 antigenic polypeptides.

### Antigens/Antigenic Polypeptides

In some embodiments, an Ebola virus or a Marburg virus antigenic polypeptide is longer than 25 amino acids and shorter than 50 amino acids. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. Polypeptides may also comprise single chain or multichain polypeptides such as antibodies or insulin and may be associated or linked. Most commonly, disulfide linkages are found in multichain polypeptides. The term polypeptide may also apply to amino acid polymers in which at least one amino acid residue is an artificial chemical analogue of a corresponding naturally-occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants possess at least 50% identity to a native or reference sequence. In some embodiments, variants share at least 80%, or at least 90% identity with a native or reference sequence.

In some embodiments “variant mimics” are provided. As used herein, the term “variant mimic” is one which contains at least one amino acid that would mimic an activated sequence. For example, glutamate may serve as a mimic for phosphoro-threonine and/or phosphoro-serine. Alternatively, variant mimics may result in deactivation or in an inactivated product containing the mimic, for example, phenylalanine may act as an inactivating substitution for tyrosine; or alanine may act as an inactivating substitution for serine.

“Orthologs” refers to genes in different species that evolved from a common ancestral gene by speciation. Normally, orthologs retain the same function in the course of evolution. Identification of orthologs is critical for reliable prediction of gene function in newly sequenced genomes.

“Analogs” is meant to include polypeptide variants which differ by one or more amino acid alterations, for example, substitutions, additions or deletions of amino acid residues that still maintain one or more of the properties of the parent or starting polypeptide.

The present disclosure provides several types of compositions that are polynucleotide or polypeptide based, including variants and derivatives. These include, for example, substitutional, insertional, deletion and covalent variants and derivatives. The term “derivative” is used synonymously with the term “variant” but generally refers to a molecule that has been modified and/or changed in any way relative to a reference molecule or starting molecule.

As such, polynucleotides encoding peptides or polypeptides containing substitutions, insertions and/or additions, deletions and covalent modifications with respect to reference sequences, in particular the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to polypeptides are those that have at least one amino acid residue in a native or starting sequence removed and a different amino acid inserted in its place at the same position. Substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Features” when referring to polypeptide or polynucleotide are defined as distinct amino acid sequence-based or nucleotide-based components of a molecule respectively. Features of the polypeptides encoded by the polynucleotides include surface manifestations, local conformational shape, folds, loops, half-loops, domains, half-domains, sites, termini or any combination thereof.

As used herein when referring to polypeptides the term “domain” refers to a motif of a polypeptide having one or more identifiable structural or functional characteristics or properties (e.g., binding capacity, serving as a site for protein-protein interactions).

As used herein when referring to polypeptides the terms “site” as it pertains to amino acid based embodiments is used synonymously with “amino acid residue” and “amino acid side chain.” As used herein when referring to polynucleotides the terms “site” as it pertains to nucleotide based embodiments is used synonymously with “nucleotide.” A site represents a position within a peptide or polypeptide or polynucleotide that may be modified, manipulated, altered, derivatized or varied within the polypeptide or polynucleotide based molecules.

As used herein the terms “termini” or “terminus” when referring to polypeptides or polynucleotides refers to an extremity of a polypeptide or polynucleotide respectively. Such extremity is not limited only to the first or final site of the polypeptide or polynucleotide but may include additional amino acids or nucleotides in the terminal regions. Polypeptide-based molecules may be characterized as having both an N-terminus (terminated by an amino acid with a free amino group (NH2)) and a C-terminus (terminated by an amino acid with a free carboxyl group (COOH)). Proteins are in some cases made up of multiple polypeptide chains brought together by disulfide bonds or by non-covalent forces (multimers, oligomers). These proteins have multiple N- and C-termini. Alternatively, the termini of the polypeptides may be modified such that they begin or end, as the case may be, with a non-polypeptide based moiety such as an organic conjugate.

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length. In another example, any protein that includes a stretch of 20, 30, 40, 50, or 100 amino acids which are 40%, 50%, 60%, 70%, 80%, 90%, 95%, or 100% identical to any of the sequences described herein can be utilized in accordance with the disclosure. In some embodiments, a polypeptide includes 2, 3, 4, 5, 6, 7, 8, 9, 10, or more mutations as shown in any of the sequences provided or referenced herein. In another example, any protein that includes a stretch of 20, 30, 40, 50, or 100 amino acids that are greater than 80%, 90%, 95%, or 100% identical to any of the sequences described herein, wherein the protein has a stretch of 5, 10, 15, 20, 25, or 30 amino acids that are less than 80%, 75%, 70%, 65% or 60% identical to any of the sequences described herein can be utilized in accordance with the disclosure.

Polypeptide or polynucleotide molecules of the present disclosure may share a certain degree of sequence similarity or identity with the reference molecules (e.g., reference polypeptides or reference polynucleotides), for example, with art-described molecules (e.g., engineered or designed molecules or wild-type molecules). The term “identity” as known in the art, refers to a relationship between the sequences of two or more polypeptides or polynucleotides, as determined by comparing the sequences. In the art, identity also means the degree of sequence relatedness between them as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453.). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm. Other tools are described herein, specifically in the definition of “identity” below.

As used herein, the term “homology” refers to the overall relatedness between polymeric molecules, e.g. between nucleic acid molecules (e.g. DNA molecules and/or RNA molecules) and/or between polypeptide molecules. Polymeric molecules (e.g. nucleic acid molecules (e.g. DNA molecules and/or RNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original one.

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

### Chemical Modifications

RNA (e.g., mRNA) vaccines of the present disclosure comprise at least one ribonucleic acid (RNA) polynucleotide having an open reading frame encoding at least one Ebola virus and/or at least one Marburg antigenic polypeptide that comprises at least one chemical modification.

The terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population. Generally, these terms do not refer to the ribonucleotide modifications in naturally occurring 5′-terminal mRNA cap moieties. With respect to a polypeptide, the term “modification” refers to a modification relative to the canonical set 20 amino acids. Polypeptides, as provided herein, are also considered “modified” of they contain amino acid substitutions, insertions or a combination of substitutions and insertions.

Polynucleotides (e.g., RNA polynucleotides, such as mRNA polynucleotides), in some embodiments, comprise various (more than one) different modifications. In some embodiments, a particular region of a polynucleotide contains one, two or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Modifications of polynucleotides include, without limitation, those described herein. Polynucleotides (e.g., RNA polynucleotides, such as mRNA polynucleotides) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone).

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

The present disclosure provides for modified nucleosides and nucleotides of a polynucleotide (e.g., RNA polynucleotides, such as mRNA polynucleotides). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphdioester linkages, in which case the polynucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into polynucleotides of the present disclosure.

The skilled artisan will appreciate that, except where otherwise noted, polynucleotide sequences set forth in the instant application will recite “T”s in a representative DNA sequence but where the sequence represents RNA, the “T”s would be substituted for “U”s. In some embodiments, modified nucleobases in nucleic acids (e.g., RNA nucleic acids, such as mRNA nucleic acids) comprise 1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a RNA nucleic acid of the disclosure comprises 1-methyl-pseudouridine (m1ψ) substitutions at one or more or all uridine positions of the nucleic acid.

In some embodiments, a RNA nucleic acid of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises pseudouridine (ψ) substitutions at one or more or all uridine positions of the nucleic acid.

In some embodiments, a RNA nucleic acid of the disclosure comprises pseudouridine (ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Flagellin Adjuvants

Flagellin is an approximately 500 amino acid monomeric protein that polymerizes to form the flagella associated with bacterial motion. Flagellin is expressed by a variety of flagellated bacteria (Salmonella typhimurium for example) as well as non-flagellated bacteria (such as Escherichia coli). Sensing of flagellin by cells of the innate immune system (dendritic cells, macrophages, etc.) is mediated by the Toll-like receptor 5 (TLRS) as well as by Nod-like receptors (NLRs) Ipaf and Naip5. TLRs and NLRs have been identified as playing a role in the activation of innate immune response and adaptive immune response. As such, flagellin provides an adjuvant effect in a vaccine.

The nucleotide and amino acid sequences encoding known flagellin polypeptides are publicly available in the NCBI GenBank database. The flagellin sequences from S. Typhimurium, H. Pylori, V. Cholera, S. marcesens, S. flexneri, T. Pallidum, L. pneumophila, B. burgdorferei, C. difficile, R. meliloti, A. tumefaciens, R. lupini, B. clarridgeiae, P. Mirabilis, B. subtilus, L. monocytogenes, P. aeruginosa, and E. coli, among others are known.

A flagellin polypeptide, as used herein, refers to a full length flagellin protein, immunogenic fragments thereof, and peptides having at least 50% sequence identify to a flagellin protein or immunogenic fragments thereof. Exemplary flagellin proteins include flagellin from Salmonella typhi (UniPro Entry number: Q56086), Salmonella typhimurium (A0A0C9DG09), Salmonella enteritidis (A0A0C9BAB7), and Salmonella choleraesuis (Q6V2X8). In some embodiments, the flagellin polypeptide has at least 60%, 70%, 75%, 80%, 90%, 95%, 97%, 98%, or 99% sequence identify to a flagellin protein or immunogenic fragments thereof.

In some embodiments, the flagellin polypeptide is an immunogenic fragment. An immunogenic fragment is a portion of a flagellin protein that provokes an immune response. In some embodiments, the immune response is a TLRS immune response. An example of an immunogenic fragment is a flagellin protein in which all or a portion of a hinge region has been deleted or replaced with other amino acids. For example, an antigenic polypeptide may be inserted in the hinge region. Hinge regions are the hypervariable regions of a flagellin. Hinge regions of a flagellin are also referred to as “D3 domain or region, “propeller domain or region,” “hypervariable domain or region” and “variable domain or region.” “At least a portion of a hinge region,” as used herein, refers to any part of the hinge region of the flagellin, or the entirety of the hinge region. In other embodiments an immunogenic fragment of flagellin is a 20, 25, 30, 35, or 40 amino acid C-terminal fragment of flagellin.

The flagellin monomer is formed by domains D0 through D3. D0 and D1, which form the stem, are composed of tandem long alpha helices and are highly conserved among different bacteria. The D1 domain includes several stretches of amino acids that are useful for TLRS activation. The entire D1 domain or one or more of the active regions within the domain are immunogenic fragments of flagellin. Examples of immunogenic regions within the D1 domain include residues 88-114 and residues 411-431 (in Salmonella typhimurium FliC flagellin. Within the 13 amino acids in the 88-100 region, at least 6 substitutions are permitted between Salmonella flagellin and other flagellins that still preserve TLRS activation. Thus, immunogenic fragments of flagellin include flagellin like sequences that activate TLRS and contain a 13 amino acid motif that is 53% or more identical to the Salmonella sequence in 88-100 of FliC (LQRVRELAVQSAN; SEQ ID NO: 183).

In some embodiments, the RNA (e.g., mRNA) vaccine includes an RNA that encodes a fusion protein of flagellin and one or more antigenic polypeptides. A “fusion protein” as used herein, refers to a linking of two components of the construct. In some embodiments, a carboxy-terminus of the antigenic polypeptide is fused or linked to an amino terminus of the flagellin polypeptide. In other embodiments, an amino-terminus of the antigenic polypeptide is fused or linked to a carboxy-terminus of the flagellin polypeptide. The fusion protein may include, for example, one, two, three, four, five, six or more flagellin polypeptides linked to one, two, three, four, five, six or more antigenic polypeptides. When two or more flagellin polypeptides and/or two or more antigenic polypeptides are linked such a construct may be referred to as a “multimer.”

Each of the components of a fusion protein may be directly linked to one another or they may be connected through a linker. For instance, the linker may be an amino acid linker. The amino acid linker encoded for by the RNA (e.g., mRNA) vaccine to link the components of the fusion protein may include, for instance, at least one member selected from the group consisting of a lysine residue, a glutamic acid residue, a serine residue and an arginine residue. In some embodiments the linker is 1-30, 1-25, 1-25, 5-10, 5, 15, or 5-20 amino acids in length.

In other embodiments the RNA (e.g., mRNA) vaccine includes at least two separate RNA polynucleotides, one encoding one or more antigenic polypeptides and the other encoding the flagellin polypeptide. The at least two RNA polynucleotides may be co-formulated in a carrier such as a lipid nanoparticle.

In Vitro Transcription of RNA (e.g., mRNA)

Ebola virus and/or Marburg virus vaccines of the present disclosure comprise at least one RNA polynucleotide, such as an mRNA (e.g., modified mRNA). mRNA, for example, is transcribed in vitro from template DNA, referred to as an “in vitro transcription template.” In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, export of the mRNA from the nucleus and translation.

In some embodiments, a polynucleotide includes 200 to 3,000 nucleotides. For example, a polynucleotide may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, Mass.), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the nucleic acids of the present invention may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, Mass.). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Methods of Treatment

Provided herein are compositions (e.g., pharmaceutical compositions), methods, kits and reagents for prevention and/or treatment of Ebola virus and/or Marburg virus in humans and other mammals. Ebola virus and/or Marburg virus RNA (e.g., mRNA) vaccines can be used as therapeutic or prophylactic agents. They may be used in medicine to prevent and/or treat infectious disease. In exemplary aspects, the Ebola virus and/or Marburg virus RNA vaccines of the present disclosure are used to provide prophylactic protection from Ebola virus. Prophylactic protection from Ebola virus and/or Marburg virus can be achieved following administration of a Ebola virus and/or Marburg virus RNA (e.g., mRNA) vaccine of the present disclosure. Vaccines can be administered once, twice, three times, four times or more but it is likely sufficient to administer the vaccine once (optionally followed by a single booster). It is possible, although less desirable, to administer the vaccine to an infected individual to achieve a therapeutic response. Dosing may need to be adjusted accordingly.

A method of eliciting an immune response in a subject against an Ebola virus and/or Marburg virus is provided in aspects of the invention. The method involves administering to the subject an Ebola virus and/or Marburg virus RNA vaccine comprising at least one RNA polynucleotide having an open reading frame encoding at least one Ebola virus and/or Marburg virus antigenic polypeptide or an immunogenic fragment thereof, thereby inducing in the subject an immune response specific to Ebola virus and/or Marburg virus antigenic polypeptide or an immunogenic fragment thereof, wherein anti-antigenic polypeptide antibody titer in the subject is increased following vaccination relative to anti-antigenic polypeptide antibody titer in a subject vaccinated with a prophylactically effective dose of a traditional vaccine against Ebola virus and/or Marburg virus. An “anti-antigenic polypeptide antibody” is a serum antibody the binds specifically to the antigenic polypeptide.

A prophylactically effective dose is a therapeutically effective dose that prevents infection with the virus at a clinically acceptable level. In some embodiments the therapeutically effective dose is a dose listed in a package insert for the vaccine. A traditional vaccine, as used herein, refers to a vaccine other than the mRNA vaccines of the invention. For instance, a traditional vaccine includes but is not limited to live microorganism vaccines, killed microorganism vaccines, subunit vaccines, protein antigen vaccines, DNA vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

In some embodiments the anti-antigenic polypeptide antibody titer in the subject is increased 1 log to 10 log following vaccination relative to anti-antigenic polypeptide antibody titer in a subject vaccinated with a prophylactically effective dose of a traditional vaccine against Ebola virus and/or Marburg virus.

In some embodiments the anti-antigenic polypeptide antibody titer in the subject is increased 1 log following vaccination relative to anti-antigenic polypeptide antibody titer in a subject vaccinated with a prophylactically effective dose of a traditional vaccine against Ebola virus and/or Marburg virus.

In some embodiments the anti-antigenic polypeptide antibody titer in the subject is increased 2 log following vaccination relative to anti-antigenic polypeptide antibody titer in a subject vaccinated with a prophylactically effective dose of a traditional vaccine against Ebola virus and/or Marburg virus.

In some embodiments the anti-antigenic polypeptide antibody titer in the subject is increased 3 log following vaccination relative to anti-antigenic polypeptide antibody titer in a subject vaccinated with a prophylactically effective dose of a traditional vaccine against Ebola virus and/or Marburg virus.

In some embodiments the anti-antigenic polypeptide antibody titer in the subject is increased 5 log following vaccination relative to anti-antigenic polypeptide antibody titer in a subject vaccinated with a prophylactically effective dose of a traditional vaccine against Ebola virus and/or Marburg virus.

In some embodiments the anti-antigenic polypeptide antibody titer in the subject is increased 10 log following vaccination relative to anti-antigenic polypeptide antibody titer in a subject vaccinated with a prophylactically effective dose of a traditional vaccine against Ebola virus and/or Marburg virus.

A method of eliciting an immune response in a subject against an Ebola virus and/or s Marburg virus is provided in other aspects of the invention. The method involves administering to the subject an Ebola virus and/or a Marburg virus RNA vaccine comprising at least one RNA polynucleotide having an open reading frame encoding at least one Ebola virus and/or Marburg virus antigenic polypeptide or an immunogenic fragment thereof, thereby inducing in the subject an immune response specific to Ebola virus and/or Marburg virus antigenic polypeptide or an immunogenic fragment thereof, wherein the immune response in the subject is equivalent to an immune response in a subject vaccinated with a traditional vaccine against the Ebola virus and/or Marburg virus at 2 times to 100 times the dosage level relative to the RNA vaccine.

In some embodiments the immune response in the subject is equivalent to an immune response in a subject vaccinated with a traditional vaccine at twice the dosage level relative to the Ebola virus and/or Marburg virus RNA vaccine.

In some embodiments the immune response in the subject is equivalent to an immune response in a subject vaccinated with a traditional vaccine at three times the dosage level relative to the Ebola virus and/or Marburg virus RNA vaccine.

In some embodiments the immune response in the subject is equivalent to an immune response in a subject vaccinated with a traditional vaccine at 4 times the dosage level relative to the Ebola virus and/or Marburg virus RNA vaccine.

In some embodiments the immune response in the subject is equivalent to an immune response in a subject vaccinated with a traditional vaccine at 5 times the dosage level relative to the Ebola virus and/or Marburg virus RNA vaccine.

In some embodiments the immune response in the subject is equivalent to an immune response in a subject vaccinated with a traditional vaccine at 10 times the dosage level relative to the Ebola virus and/or Marburg virus RNA vaccine.

In some embodiments the immune response in the subject is equivalent to an immune response in a subject vaccinated with a traditional vaccine at 50 times the dosage level relative to the Ebola virus and/or Marburg virus RNA vaccine.

In some embodiments the immune response in the subject is equivalent to an immune response in a subject vaccinated with a traditional vaccine at 100 times the dosage level relative to the Ebola virus and/or Marburg virus RNA vaccine.

In some embodiments the immune response in the subject is equivalent to an immune response in a subject vaccinated with a traditional vaccine at 10 times to 1000 times the dosage level relative to the Ebola virus and/or Marburg virus RNA vaccine.

In some embodiments the immune response in the subject is equivalent to an immune response in a subject vaccinated with a traditional vaccine at 100 times to 1000 times the dosage level relative to the Ebola virus and/or Marburg virus RNA vaccine.

In other embodiments the immune response is assessed by determining anti-antigenic polypeptide antibody titer in the subject.

In other aspects the invention is a method of eliciting an immune response in a subject against a Ebola virus and/or Marburg virus by administering to the subject an Ebola virus and/or a Marburg virus RNA vaccine comprising at least one RNA polynucleotide having an open reading frame encoding at least one Ebola virus and/or Marburg virus antigenic polypeptide or an immunogenic fragment thereof, thereby inducing in the subject an immune response specific to Ebola virus and/or Marburg virus antigenic polypeptide or an immunogenic fragment thereof, wherein the immune response in the subject is induced 2 days to 10 weeks earlier relative to an immune response induced in a subject vaccinated with a prophylactically effective dose of a traditional vaccine against the Ebola virus and/or the Marburg virus. In some embodiments the immune response in the subject is induced in a subject vaccinated with a prophylactically effective dose of a traditional vaccine at 2 times to 100 times the dosage level relative to the RNA vaccine.

In some embodiments the immune response in the subject is induced 2 days earlier relative to an immune response induced in a subject vaccinated with a prophylactically effective dose of a traditional vaccine.

In some embodiments the immune response in the subject is induced 3 days earlier relative to an immune response induced in a subject vaccinated a prophylactically effective dose of a traditional vaccine.

In some embodiments the immune response in the subject is induced 1 week earlier relative to an immune response induced in a subject vaccinated with a prophylactically effective dose of a traditional vaccine.

In some embodiments the immune response in the subject is induced 2 weeks earlier relative to an immune response induced in a subject vaccinated with a prophylactically effective dose of a traditional vaccine.

In some embodiments the immune response in the subject is induced 3 weeks earlier relative to an immune response induced in a subject vaccinated with a prophylactically effective dose of a traditional vaccine.

In some embodiments the immune response in the subject is induced 5 weeks earlier relative to an immune response induced in a subject vaccinated with a prophylactically effective dose of a traditional vaccine.

In some embodiments the immune response in the subject is induced 10 weeks earlier relative to an immune response induced in a subject vaccinated with a prophylactically effective dose of a traditional vaccine.

A method of eliciting an immune response in a subject against an Ebola virus and/or a Marburg virus by administering to the subject an Ebola virus and/or Marburg virus RNA vaccine having an open reading frame encoding a first antigenic polypeptide, wherein the RNA polynucleotide does not include a stabilization element, and wherein an adjuvant is not coformulated or co-administered with the vaccine.

Broad Spectrum RNA (e.g., mRNA) Vaccines

There may be situations where persons are at risk for infection with more than one strain of Ebola virus and/or Marburg virus. RNA (mRNA) therapeutic vaccines are particularly amenable to combination vaccination approaches due to a number of factors including, but not limited to, speed of manufacture, ability to rapidly tailor vaccines to accommodate perceived geographical threat, and the like. Moreover, because the vaccines utilize the human body to produce the antigenic protein, the vaccines are amenable to the production of larger, more complex antigenic proteins, allowing for proper folding, surface expression, antigen presentation, etc. in the human subject. To protect against more than one strain of Ebola virus and/or Marburg virus, a combination vaccine can be administered that includes RNA encoding at least one antigenic polypeptide protein (or antigenic portion thereof) of a first Ebola virus and/or Marburg virus antigen and further includes RNA encoding at least one antigenic polypeptide protein (or antigenic portion thereof) of a second Ebola virus and/or Marburg virus antigen. Additionally, or alternatively an epitope may be selected that has cross-strain homology and thus produces an immune response against more than one strain. RNAs (mRNAs) can be co-formulated, for example, in a single lipid nanoparticle (LNP) or can be formulated in separate LNPs destined for co-administration.

### Therapeutic and Prophylactic Compositions

Provided herein are compositions (e.g., pharmaceutical compositions), methods, kits and reagents for prevention, treatment or diagnosis of Ebola virus and/or Marburg virus in humans and other mammals, for example. Ebola virus and/or Marburg virus RNA (e.g., mRNA) vaccines can be used as therapeutic or prophylactic agents. They may be used in medicine to prevent and/or treat infectious disease. In some embodiments, the Ebola virus and/or Marburg virus vaccines of the invention can be envisioned for use in the priming of immune effector cells, for example, to activate peripheral blood mononuclear cells (PBMCs) ex vivo, which are then infused (re-infused) into a subject.

In exemplary embodiments, an Ebola virus and/or Marburg virus vaccine containing RNA polynucleotides as described herein can be administered to a subject (e.g., a mammalian subject, such as a human subject), and the RNA polynucleotides are translated in vivo to produce an antigenic polypeptide.

The Ebola virus and/or Marburg virus RNA vaccines may be induced for translation of a polypeptide (e.g., antigen or immunogen) in a cell, tissue or organism. In exemplary embodiments, such translation occurs in vivo, although there can be envisioned embodiments where such translation occurs ex vivo, in culture or in vitro. In exemplary embodiments, the cell, tissue or organism is contacted with an effective amount of a composition containing an Ebola virus and/or Marburg virus RNA vaccine that contains a polynucleotide that has at least one a translatable region encoding an antigenic polypeptide.

An “effective amount” of an Ebola virus and/or Marburg virus RNA vaccine is provided based, at least in part, on the target tissue, target cell type, means of administration, physical characteristics of the polynucleotide (e.g., size, and extent of modified nucleosides) and other components of the Ebola virus and/or Marburg virus RNA vaccine, and other determinants. In general, an effective amount of the Ebola virus and/or Marburg virus RNA vaccine composition provides an induced or boosted immune response as a function of antigen production in the cell, preferably more efficient than a composition containing a corresponding unmodified polynucleotide encoding the same antigen or a peptide antigen. Increased antigen production may be demonstrated by increased cell transfection (the percentage of cells transfected with the RNA vaccine), increased protein translation from the polynucleotide, decreased nucleic acid degradation (as demonstrated, for example, by increased duration of protein translation from a modified polynucleotide), or altered antigen specific immune response of the host cell.

In some embodiments, RNA vaccines (including polynucleotides their encoded polypeptides) in accordance with the present disclosure may be used for treatment of Ebola virus and/or Marburg virus.

Ebola virus and/or Marburg virus RNA vaccines may be administered prophylactically or therapeutically as part of an active immunization scheme to healthy individuals or early in infection during the incubation phase or during active infection after onset of symptoms. In some embodiments, the amount of RNA vaccines of the present disclosure provided to a cell, a tissue or a subject may be an amount effective for immune prophylaxis.

Ebola virus and/or Marburg virus RNA vaccines may be administrated with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In exemplary embodiments, the time of administration between the initial administration of the prophylactic composition and the booster may be, but is not limited to, 1 week, 2 weeks, 3 weeks, 1 month, 2 months, 3 months, 6 months or 1 year.

In some embodiments, Ebola virus and/or Marburg virus RNA vaccines may be administered intramuscularly or intradermally, similarly to the administration of inactivated vaccines known in the art.

Ebola virus and/or Marburg virus RNA vaccines may be utilized in various settings depending on the prevalence of the infection or the degree or level of unmet medical need. As a non-limiting example, the RNA vaccines may be utilized to treat and/or prevent a variety of infectious disease. RNA vaccines have superior properties in that they produce much larger antibody titers and produce responses early than commercially available anti-virals.

Provided herein are pharmaceutical compositions including Ebola virus and/or Marburg virus RNA vaccines and RNA vaccine compositions and/or complexes optionally in combination with one or more pharmaceutically acceptable excipients.

Ebola virus and/or Marburg virus RNA vaccines may be formulated or administered alone or in conjunction with one or more other components. In some embodiments, Ebola virus and/or Marburg virus RNA vaccines do not include an adjuvant (they are adjuvant free).

Ebola virus and/or Marburg virus RNA vaccines may be formulated or administered in combination with one or more pharmaceutically-acceptable excipients. In some embodiments, vaccine compositions comprise at least one additional active substances, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, Ebola virus and/or Marburg virus RNA vaccines are administered to humans, human patients or subjects. For the purposes of the present disclosure, the phrase “active ingredient” generally refers to the RNA vaccines or the polynucleotides contained therein, for example, RNA polynucleotides (e.g., mRNA polynucleotides) encoding antigenic polypeptides.

Formulations of the vaccine compositions described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

Ebola virus and/or Marburg virus RNA vaccines can be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with Ebola virus and/or Marburg virus RNA vaccines (e.g., for transplantation into a subject), hyaluronidase, nanoparticle mimics and combinations thereof.

### Stabilizing Elements

Naturally-occurring eukaryotic mRNA molecules have been found to contain stabilizing elements, including, but not limited to untranslated regions (UTR) at their 5′-end (5′UTR) and/or at their 3′-end (3′UTR), in addition to other structural features, such as a 5′-cap structure or a 3′-poly(A) tail. Both the 5′UTR and the 3′UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing. The 3′-poly(A) tail is typically a stretch of adenine nucleotides added to the 3′-end of the transcribed mRNA. It can comprise up to about 400 adenine nucleotides. In some embodiments the length of the 3′-poly(A) tail may be an essential element with respect to the stability of the individual mRNA.

In some embodiments the RNA vaccine may include one or more stabilizing elements. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it is peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, the RNA vaccines include a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine:guanine phosphoribosyl transferase (GPT)).

In some embodiments, the combination of a poly(A) sequence or polyadenylation signal and at least one histone stem-loop, even though both represent alternative mechanisms in nature, acts synergistically to increase the protein expression beyond the level observed with either of the individual elements. It has been found that the synergistic effect of the combination of poly(A) and at least one histone stem-loop does not depend on the order of the elements or the length of the poly(A) sequence.

In some embodiments, the RNA vaccine does not comprise a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. Ideally, the inventive nucleic acid does not include an intron.

In some embodiments, the RNA vaccine may or may not contain a enhancer and/or promoter sequence, which may be modified or unmodified or which may be activated or inactivated. In some embodiments, the histone stem-loop is generally derived from histone genes, and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures, but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In other embodiments the RNA vaccine may have one or more AU-rich sequences removed. These sequences, sometimes referred to as AURES are destabilizing sequences found in the 3′UTR. The AURES may be removed from the RNA vaccines. Alternatively the AURES may remain in the RNA vaccine.

### Signal Peptides

In some embodiments, an EBOV or MARV vaccine comprises a RNA having an ORF that encodes a signal peptide fused to the EBOV or MARV antigen. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by a ER-resident signal peptidase of the host cell, or they remain uncleaved and function as a membrane anchor. A signal peptide may also facilitate the targeting of the protein to the cell membrane.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than EBOV or MARV antigens in nature) are known in the art and can be tested for desired properties and then incorporated into a nucleic acid of the disclosure. In some embodiments, the signal peptide is selected from: a HuIgGk signal peptide (METPAQLLFLLLLWLPDTTG; SEQ ID NO: 178); IgE heavy chain epsilon-1 signal peptide (MDWTWILFLVAAATRVHS; SEQ ID NO: 179); Japanese encephalitis PRM signal sequence (MLGSNSGQRVVFTILLLLVAPAYS; SEQ ID NO: 180), VSVg protein signal sequence (MKCLLYLAFLFIGVNCA; SEQ ID NO: 181) and Japanese encephalitis JEV signal sequence (MWLVSLAIVTACAGA; SEQ ID NO: 182).

In some embodiments, the signal peptide is fused to the N-terminus of at least one antigenic polypeptide. In some embodiments, a signal peptide is fused to the C-terminus of at least one antigenic polypeptide.

### Fusion Proteins

In some embodiments, an EBOV or MARV RNA vaccine of the present disclosure includes an RNA encoding an antigenic fusion protein. Thus, the encoded antigen or antigens may include two or more proteins (e.g., protein and/or protein fragment) joined together. Alternatively, the protein to which a protein antigen is fused does not promote a strong immune response to itself, but rather to the EBOV or MARV antigen. Antigenic fusion proteins, in some embodiments, retain the functional property from each original protein.

### Scaffold Moieties

The RNA (e.g., mRNA) vaccines as provided herein, in some embodiments, encode fusion proteins which comprise EBOV or MARV antigens linked to scaffold moieties. In some embodiments, such scaffold moieties impart desired properties to an antigen encoded by a nucleic acid of the disclosure. For example scaffold proteins may improve the immunogenicity of an antigen, e.g., by altering the structure of the antigen, altering the uptake and processing of the antigen, and/or causing the antigen to bind to a binding partner.

In some embodiments, the scaffold moiety is protein that can self-assemble into protein nanoparticles that are highly symmetric, stable, and structurally organized, with diameters of 10-150 nm, a highly suitable size range for optimal interactions with various cells of the immune system. In one embodiment, viral proteins or virus-like particles can be used to form stable nanoparticle structures. Examples of such viral proteins are known in the art. For example, in some embodiments, the scaffold moiety is a hepatitis B surface antigen (HBsAg). HBsAg forms spherical particles with an average diameter of ˜22 nm and which lacked nucleic acid and hence are non-infectious (Lopez-Sagaseta, J. et al. Computational and Structural Biotechnology Journal 14 (2016) 58-68). In some embodiments, the scaffold moiety is a hepatitis B core antigen (HBcAg) self-assembles into particles of 24-31 nm diameter, which resembled the viral cores obtained from HBV-infected human liver. HBcAg produced in self-assembles into two classes of differently sized nanoparticles of 300 Å and 360 A diameter, corresponding to 180 or 240 protomers. In some embodiments an EBOV or MARV antigen is fused to HBsAG or HBcAG to facilitate self-assembly of nanoparticles displaying the EBOV or MARV antigen.

In another embodiment, bacterial protein platforms may be used. Non-limiting examples of these self-assembling proteins include ferritin, lumazine and encapsulin.

Ferritin is a protein whose main function is intracellular iron storage. Ferritin is made of 24 subunits, each composed of a four-alpha-helix bundle, that self-assemble in a quaternary structure with octahedral symmetry (Cho K. J. et al. J Mol Biol. 2009; 390:83-98). Several high-resolution structures of ferritin have been determined, confirming that Helicobacter pylori ferritin is made of 24 identical protomers, whereas in animals, there are ferritin light and heavy chains that can assemble alone or combine with different ratios into particles of 24 subunits (Granier T. et al. J Biol Inorg Chem. 2003; 8:105-111; Lawson D. M. et al. Nature. 1991; 349:541-544). Ferritin self-assembles into nanoparticles with robust thermal and chemical stability. Thus, the ferritin nanoparticle is well-suited to carry and expose antigens.

Lumazine synthase (LS) is also well-suited as a nanoparticle platform for antigen display. LS, which is responsible for the penultimate catalytic step in the biosynthesis of riboflavin, is an enzyme present in a broad variety of organisms, including archaea, bacteria, fungi, plants, and eubacteria (Weber S. E. Flavins and Flavoproteins. Methods and Protocols, Series: Methods in Molecular Biology. 2014). The LS monomer is 150 amino acids long, and consists of beta-sheets along with tandem alpha-helices flanking its sides. A number of different quaternary structures have been reported for LS, illustrating its morphological versatility: from homopentamers up to symmetrical assemblies of 12 pentamers forming capsids of 150 Å diameter. Even LS cages of more than 100 subunits have been described (Zhang X. et al. J Mol Biol. 2006; 362:753-770).

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers having a thin and icosahedral T=1 symmetric cage structure with interior and exterior diameters of 20 and 24 nm, respectively (Sutter M. et al. Nat Struct Mol Biol. 2008, 15: 939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses (Rahmanpour R. et al. FEBS J. 2013, 280: 2097-2104).

### Linkers and Cleavable Peptides

In some embodiments, the mRNAs of the disclosure encode more than one polypeptide, referred to herein as fusion proteins. In some embodiments, the mRNA further encodes a linker located between at least one or each domain of the fusion protein. The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2A peptides, has been described in the art (see for example, Kim, J. H. et al. (2011) PLoS ONE 6:e18556). In some embodiments, the linker is an F2A linker. In some embodiments, the linker is a GGGS linker. In some embodiments, the fusion protein contains three domains with intervening linkers, having the structure: domain-linker-domain-linker-domain.

Cleavable linkers known in the art may be used in connection with the disclosure. Exemplary such linkers include: F2A linkers, T2A linkers, P2A linkers, E2A linkers (See, e.g., WO2017127750). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one antigen/polypeptide separately within the same molecule) may be suitable for use as provided herein.

### Sequence Optimization

In one embodiment, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park Calif.) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding an EBOV or MARV antigen). In some embodiments, a codon optimized sequence shares less than 90% sequence identity to a naturally-occurring or wild-type sequence (e.g., a naturally-occurring or wild-type mRNA sequence encoding n EBOV or MARV antigen). In some embodiments, a codon optimized sequence shares less than 85% sequence identity to a naturally-occurring or wild-type sequence (e.g., a naturally-occurring or wild-type mRNA sequence encoding an EBOV or MARV antigen). In some embodiments, a codon optimized sequence shares less than 80% sequence identity to a naturally-occurring or wild-type sequence (e.g., a naturally-occurring or wild-type mRNA sequence encoding an EBOV or MARV antigen). In some embodiments, a codon optimized sequence shares less than 75% sequence identity to a naturally-occurring or wild-type sequence (e.g., a naturally-occurring or wild-type mRNA sequence encoding an EBOV or MARV antigen).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding anEBOV or MARV antigen). In some embodiments, a codon optimized sequence shares between 65% and 75% or about 80% sequence identity to a naturally-occurring or wild-type sequence (e.g., a naturally-occurring or wild-type mRNA sequence encoding an EBOV or MARV antigen).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than an EBOV or MARV antigen encoded by a non-codon-optimized)sequence.

When transfected into mammalian cells, the modified mRNAs have a stability of between 12-18 hours, or greater than 18 hours, e.g., 24, 36, 48, 60, 72, or greater than 72 hours.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemically Unmodified Nucleotides

In some embodiments, at least one RNA (e.g., mRNA) of an EBOV or MARV vaccines of the present disclosure is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

### Nanoparticle Formulations

In some embodiments, Ebola virus and/or Marburg virus RNA (e.g., mRNA) vaccines are formulated in a nanoparticle. In some embodiments, Ebola virus and/or Marburg virus RNA vaccines are formulated in a lipid nanoparticle.

Vaccines of the present disclosure are typically formulated in lipid nanoparticle. In some embodiments, the lipid nanoparticle comprises at least one ionizable cationic lipid, at least one non-cationic lipid, at least one sterol, and/or at least one polyethylene glycol (PEG)-modified lipid.

In some embodiments, the lipid nanoparticle comprises a molar ratio of 20-60% ionizable cationic lipid. For example, the lipid nanoparticle may comprise a molar ratio of 20-50%, 20-40%, 20-30%, 30-60%, 30-50%, 30-40%, 40-60%, 40-50%, or 50-60% ionizable cationic lipid. In some embodiments, the lipid nanoparticle comprises a molar ratio of 20%, 30%, 40%, 50, or 60% ionizable cationic lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or 25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol. In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable cationic lipid, 5-25% non-cationic lipid, 25-55% sterol, and 0.5-15% PEG-modified lipid.

In some embodiments, an ionizable cationic lipid of the disclosure comprises a compound of Formula (I):

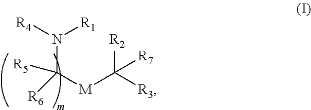

or a salt or isomer thereof, wherein:

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—,

—N(R′)C(O)—, —C(O)—, —C(S)—, —C(S)S—, —SC(S)—, —CH(OH)—, —P(O)(OR′)O—, —S(O)₂—, —S —S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and

C₃₋₁₄ alkenyl;

each R* is independently selected from the group consisting of C₁₋₁₂ alkyl and

C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13.

In some embodiments, a subset of compounds of Formula (I) includes those in which when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR,

—CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR,
—O(CH₂)—N(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13, or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆

carbocycle, —(CH₂)ₙQ, —(CH₂)ₙCHQR,
—CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR,
—O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR,
and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆

carbocycle, —(CH₂)ₙQ, —(CH₂)—CHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR,
—O(CH₂)—N(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is —(CH₂)ₙQ or —(CH₂)ₙCHQR, where Q is —N(R)₂, and n is selected from 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₁₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13, or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₁₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or isomers thereof.

In some embodiments, a subset of compounds of Formula (I) includes those of Formula (IA):

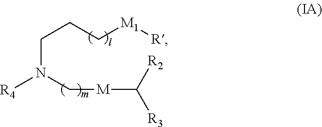

or a salt or isomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (II):

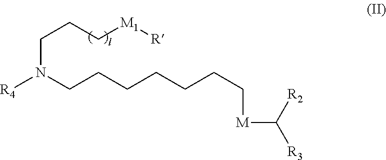

or a salt or isomer thereof, wherein 1 is selected from 1, 2, 3, 4, and 5; M₁ is a bond or M′; R₄ is unsubstituted C₁₋₃ alkyl, or —(CH₂)ₙQ, in which n is 2, 3, or 4, and Q is
OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected
from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (IIa), (IIb), (IIc), or (IIe):

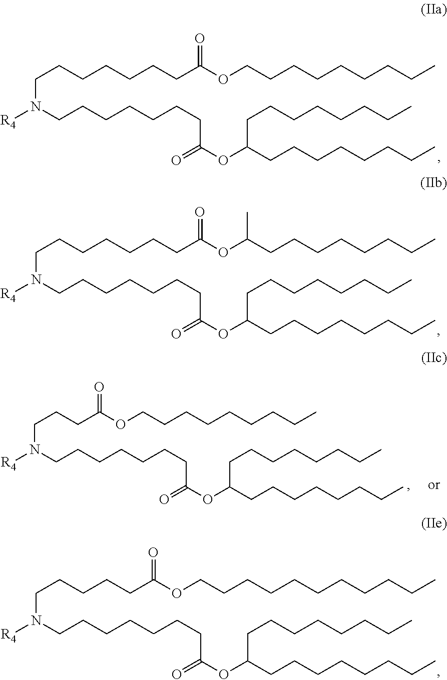

or a salt or isomer thereof, wherein R₄ is as described herein.

In some embodiments, a subset of compounds of Formula (I) includes those of Formula (IId):

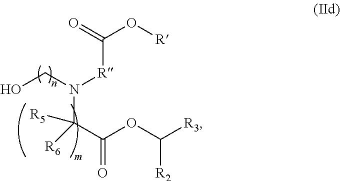

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable cationic lipid of the disclosure comprises a compound having structure:

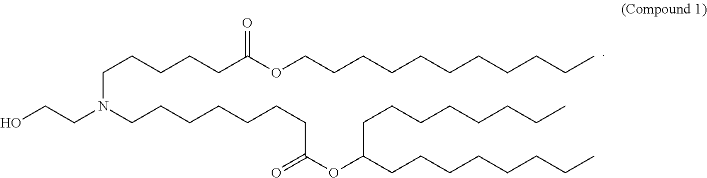 [SMILES: CCCCCCCCCCCOC(=O)CCCCCN(CCO)CCCCCCCC(=O)OC(CCCCCCCC)CCCCCCCC]

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid of the disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, a sterol of the disclosure comprises cholesterol, fecosterol, sitosterol, ergosterol, campesterol, stigmasterol, bras sicasterol, tomatidine, ursolic acid, alpha-tocopherol, and mixtures thereof.

In some embodiments, a LNP of the disclosure comprises an ionizable cationic lipid of Compound 1, wherein the non-cationic lipid is DSPC, the structural lipid that is cholesterol, and the PEG lipid is PEG-DMG.

In some embodiments, a LNP of the disclosure comprises an N:P ratio of from about 2:1 to about 30:1.

In some embodiments, a LNP of the disclosure comprises an N:P ratio of about 6:1.

In some embodiments, a LNP of the disclosure comprises an N:P ratio of about 3:1.

In some embodiments, a LNP of the disclosure comprises a wt/wt ratio of the ionizable cationic lipid component to the RNA of from about 10:1 to about 100:1.

In some embodiments, a LNP of the disclosure comprises a wt/wt ratio of the ionizable cationic lipid component to the RNA of about 20:1.

In some embodiments, a LNP of the disclosure comprises a wt/wt ratio of the ionizable cationic lipid component to the RNA of about 10:1.

In some embodiments, a LNP of the disclosure has a mean diameter from about 50 nm to about 150 nm.

In some embodiments, a LNP of the disclosure has a mean diameter from about 70 nm to about 120 nm.

### Multivalent Vaccines

The EBOV and/or MARV vaccines, as provided herein, may include an RNA (e.g. mRNA) or multiple RNAs encoding two or more antigens of the same EBOV orMARV species. In some embodiments, a EBOV and/or MARV vaccine includes an RNA or multiple RNAs encoding two or more antigens selected from glycoprotein (GP), surface EBOV GP, wild type EBOV GP, sGP, delta peptide (Δ-peptide), GP1, GP1,2Δ and/or a MARV glycoprotein (GP) antigens. In some embodiments, the RNA (at least one RNA) of a EBOV and/or MARV vaccine may encode 1, 2, 3, 4, 5, 6, 7, 8, 9, 10, 11, 12, or more antigens.

In some embodiments, a EBOV and/or MARV vaccine comprises at least one, two or three RNA encoding a MARV GP antigen.

In some embodiments, a EBOV and/or MARV vaccine comprises at least one, two or three RNA encoding a EBOV GP antigens.

In some embodiments, a EBOV and/or MARV vaccine comprises at least one RNA encoding EBOV antigens and at least one RNA encoding MARV antigens.

In some embodiments, a EBOV and/or MARV vaccine comprises at least one RNA encoding EBOV and MARV antigens.

In some embodiments, two or more different RNA (e.g., mRNA) encoding antigens may be formulated in the same lipid nanoparticle. In other embodiments, two or more different RNA encoding antigens may be formulated in separate lipid nanoparticles (each RNA formulated in a single lipid nanoparticle). The lipid nanoparticles may then be combined and administered as a single vaccine composition (e.g., comprising multiple RNA encoding multiple antigens) or may be administered separately.

### Combination Vaccines

The EBOV and/or MARV vaccines, as provided herein, may include an RNA or multiple RNAs encoding two or more antigens of the same or different EBOV and/or MARV species. Also provided herein are combination vaccines that include RNA encoding one or more EBOV and/or MARV antigen(s) and one or more antigen(s) of a different organisms (e.g., bacterial and/or viral organism). Thus, the vaccines of the present disclosure may be combination vaccines that target one or more antigens of the same species, or one or more antigens of different species, e.g., antigens which induce immunity to organisms which are found in the same geographic areas where the risk of EBOV and/or MARV infection is high or organisms to which an individual is likely to be exposed to when exposed to EBOV and/or MARV.

### Modes of Vaccine Administration

Ebola virus and/or Marburg virus RNA vaccines may be administered by any route which results in a therapeutically effective outcome. These include, but are not limited, to intradermal, intramuscular, and/or subcutaneous administration. The present disclosure provides methods comprising administering RNA vaccines to a subject in need thereof. The exact amount required will vary from subject to subject, depending on the species, age, and general condition of the subject, the severity of the disease, the particular composition, its mode of administration, its mode of activity, and the like. Ebola virus and/or Marburg virus RNA vaccines compositions are typically formulated in dosage unit form for ease of administration and uniformity of dosage. It will be understood, however, that the total daily usage of Ebola virus and/or Marburg virus RNA vaccines compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, Ebola virus and/or Marburg virus RNA vaccines compositions may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see e.g., the range of unit doses described in International Publication No WO2013078199, herein incorporated by reference in its entirety). The desired dosage may be delivered three times a day, two times a day, once a day, every other day, every third day, every week, every two weeks, every three weeks, every four weeks, every 2 months, every three months, every 6 months, etc. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In exemplary embodiments, Ebola virus and/or Marburg virus RNA vaccines compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg.

In some embodiments, Ebola virus and/or Marburg virus RNA vaccine compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, Ebola virus and/or Marburg virus RNA vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.0100 mg, 0.025 mg, 0.050 mg, 0.075 mg, 0.100 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, a Ebola virus and/or Marburg virus RNA vaccine composition may be administered three or four times.

In some embodiments, Ebola virus and/or Marburg virus RNA vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.010 mg, 0.025 mg, 0.100 mg or 0.400 mg.

In some embodiments the RNA vaccine for use in a method of vaccinating a subject is administered the subject a single dosage of between 10 μg/kg and 400 μg/kg of the nucleic acid vaccine in an effective amount to vaccinate the subject. In some embodiments the RNA vaccine for use in a method of vaccinating a subject is administered the subject a single dosage of between 10 μg and 400 μg of the nucleic acid vaccine in an effective amount to vaccinate the subject.

A RNA vaccine pharmaceutical composition described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

Some aspects of the present disclosure provide formulations of the Ebola virus and/or Marburg virus RNA (e.g., mRNA) vaccine, wherein the Ebola virus and/or Marburg virus RNA vaccine is formulated in an effective amount to produce an antigen specific immune response in a subject (e.g., production of antibodies specific to an anti-Ebola virus antigenic polypeptide and/or an anti-Marburg virus antigenic polypeptide). “An effective amount” is a dose of an Ebola virus and/or Marburg virus RNA (e.g., mRNA) vaccine effective to produce an antigen-specific immune response. Also provided herein are methods of inducing an antigen-specific immune response in a subject.

In some embodiments, the antigen-specific immune response is characterized by measuring an anti-Ebola virus and/or an anti-Marburg virus antigenic polypeptide antibody titer produced in a subject administered a Ebola virus and/or Marburg virus RNA (e.g., mRNA) vaccine as provided herein. An antibody titer is a measurement of the amount of antibodies within a subject, for example, antibodies that are specific to a particular antigen (e.g., an anti-Ebola virus and/or anti-Marburg virus antigenic polypeptide) or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has had an infection or to determine whether immunizations are required. In some embodiements, an antibody titer is used to determine the strength of an autoimmune response, to determine whether a booster immunization is needed, to derermine whether a previous vaccine was effective, and to identify any recent or prior infections. In accordance with the present disclosure, an antibody titer may be used to determine the strength of an immune response induced in a subject by the Ebola virus and/or Marburg virus RNA vaccine.

In some embodiments, an anti-Ebola virus and/or an anti-Marburg virus antigenic polypeptide antibody titer produced in a subject is increased by at least 1 log relative to a control. For example, anti-antigenic polypeptide antibody titer produced in a subject may be increased by at least 1.5, at least 2, at least 2.5, or at least 3 log relative to a control. In some embodiments, the anti-Ebola virus and/or anti-Marburg virus antigenic polypeptide antibody titer produced in the subject is increased by 1, 1.5, 2, 2.5 or 3 log relative to a control. In some embodiments, the anti-Ebola virus and/or anti-Marburg virus antigenic polypeptide antibody titer produced in the subject is increased by 1-3 log relative to a control. For example, the anti-Ebola virus and/or anti-Marburg virus antigenic polypeptide antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, the anti-Ebola virus and/or anti-Marburg virus antigenic polypeptide antibody titer produced in a subject is increased at least 2 times relative to a control. For example, the anti-Ebola virus and/or anti-Marburg virus antigenic polypeptide antibody titer produced in a subject may be increased at least 3 times, at least 4 times, at least 5 times, at least 6 times, at least 7 times, at least 8 times, at least 9 times, or at least 10 times relative to a control. In some embodiments, the anti-Ebola virus and/or anti-Marburg virus antigenic polypeptide antibody titer produced in the subject is increased 2, 3, 4, 5, 6, 7, 8, 9, or 10 times relative to a control. In some embodiments, the anti-Ebola virus and/or anti-Marburg virus antigenic polypeptide antibody titer produced in a subject is increased 2-10 times relative to a control. For example, the anti-Ebola virus and/or anti-Marburg virus antigenic polypeptide antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

A control, in some embodiments, is the anti-Ebola virus and/or anti-Marburg virus antigenic polypeptide antibody titer produced in a subject who has not been administered a Ebola virus and/or Marburg virus RNA (e.g., mRNA) vaccine. In some embodiments, a control is an anti-Ebola virus and/or anti-Marburg virus antigenic polypeptide antibody titer produced in a subject who has been administered a live attenuated Ebola virus and/or Marburg virus vaccine. An attenuated vaccine is a vaccine produced by reducing the virulence of a viable (live). An attenuated virus is altered in a manner that renders it harmless or less virulent relative to live, unmodified virus. In some embodiments, a control is an anti-Ebola virus and/or anti-Marburg virus antigenic polypeptide antibody titer produced in a subject administered inactivated Ebola virus and/or Marburg virus vaccine. In some embodiments, a control is an anti-Ebola virus and/or anti-Marburg virus antigenic polypeptide antibody titer produced in a subject administered a recombinant or purified Ebola virus and/or Marburg virus protein vaccine. Recombinant protein vaccines typically include protein antigens that either have been produced in a heterologous expression system (e.g., bacteria or yeast) or purified from large amounts of the pathogenic organism. In other embodiments the control is ChAd3-EBO-Z, Chimpanzee adenovirus vector for single IM dose by GSK. In other embodiments the control is VSV-EBOV, a recombinant, replication-competent vaccine, consisting of a vesicular stomatitis virus, which has been genetically engineered to express Ebola and/or Marburg glycoproteins so as to provoke an immune response against the complete Ebola virus and/or Marburg virus.

In some embodiments the vaccination protocol is a ring vaccination. A ring vaccination vaccinates all suspected individuals in an area around an outbreak (e.g., family members of those infected).

In some embodiments, an effective amount of an Ebola virus and/or Marburg virus RNA (e.g., mRNA) vaccine is a dose that is reduced compared to the standard of care dose of a recombinant Ebola virus and/or Marburg virus protein vaccine. A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as provided herein, refers to the dose of a recombinant or purified Ebola virus and/or Marburg virus protein vaccine, or a live attenuated or inactivated Ebola virus and/or Marburg virus vaccine, that a physician/clinician or other medical professional would administer to a subject to treat or prevent Ebola virus and/or Marburg virus, or a Ebola virus-related and/or Marburg virus-related condition, while following the standard of care guideline for treating or preventing Ebola virus and/or Marburg virus, or a Ebola virus-related and/or Marburg virus-related condition.

In some embodiments, the anti-Ebola virus and/or anti-Marburg virus antigenic polypeptide antibody titer produced in a subject administered an effective amount of a Ebola virus and/or Marburg virus RNA vaccine is equivalent to an anti-Ebola virus and/or anti-Marburg virus antigenic polypeptide antibody titer produced in a control subject administered a standard of care dose of a recombinant or purified Ebola virus and/or Marburg virus protein vaccine or a live attenuated or inactivated Ebola virus and/or Marburg virus vaccine.

In some embodiments, an effective amount of a Ebola virus and/or Marburg virus RNA (e.g., mRNA) vaccine is a dose equivalent to an at least 2-fold reduction in a standard of care dose of a recombinant or purified Ebola virus and/or Marburg virus protein vaccine. For example, an effective amount of a Ebola virus and/or Marburg virus RNA vaccine may be a dose equivalent to an at least 3-fold, at least 4-fold, at least 5-fold, at least 6-fold, at least 7-fold, at least 8-fold, at least 9-fold, or at least 10-fold reduction in a standard of care dose of a recombinant or purified Ebola virus and/or Marburg virus protein vaccine. In some embodiments, an effective amount of a Ebola virus and/or Marburg virus RNA vaccine is a dose equivalent to an at least at least 100-fold, at least 500-fold, or at least 1000-fold reduction in a standard of care dose of a recombinant or purified Ebola virus and/or Marburg virus protein vaccine. In some embodiments, an effective amount of a Ebola virus and/or Marburg virus RNA vaccine is a dose equivalent to a 2-, 3-, 4-, 5-, 6-, 7-, 8-, 9-, 10-, 20-, 50-, 100-, 250-, 500-, or 1000-fold reduction in a standard of care dose of a recombinant or purified Ebola virus and/or Marburg virus protein vaccine. In some embodiments, the anti-Ebola virus and/or anti-Marburg virus antigenic polypeptide antibody titer produced in a subject administered an effective amount of a Ebola virus and/or Marburg virus RNA vaccine is equivalent to an anti-Ebola virus and/or anti-Marburg virus antigenic polypeptide antibody titer produced in a control subject administered the standard of care dose of a recombinant or protein Ebola virus and/or Marburg virus protein vaccine or a live attenuated or inactivated Ebola virus and/or Marburg virus vaccine. In some embodiments, an effective amount of a Ebola virus and/or Marburg virus RNA (e.g., mRNA) vaccine is a dose equivalent to a 2-fold to 1000-fold (e.g., 2-fold to 100-fold, 10-fold to 1000-fold) reduction in the standard of care dose of a recombinant or purified Ebola virus and/or Marburg virus protein vaccine, wherein the anti-Ebola virus and/or anti-Marburg virus antigenic polypeptide antibody titer produced in the subject is equivalent to an anti-Ebola virus and/or anti-Marburg virus antigenic polypeptide antibody titer produced in a control subject administered the standard of care dose of a recombinant or purified Ebola virus and/or Marburg virus protein vaccine or a live attenuated or inactivated Ebola virus and/or Marburg virus vaccine.

In some embodiments, the effective amount of a Ebola virus and/or Marburg virus RNA (e.g., mRNA) vaccine is a dose equivalent to a 2 to 1000-, 2 to 900-, 2 to 800-, 2 to 700-, 2 to 600-, 2 to 500-, 2 to 400-, 2 to 300-, 2 to 200-, 2 to 100-, 2 to 90-, 2 to 80-, 2 to 70-, 2 to 60-, 2 to 50-, 2 to 40-, 2 to 30-, 2 to 20-, 2 to 10-, 2 to 9-, 2 to 8-, 2 to 7-, 2 to 6-, 2 to 5-, 2 to 4-, 2 to 3-, 3 to 1000-, 3 to 900-, 3 to 800-, 3 to 700-, 3 to 600-, 3 to 500-, 3 to 400-, 3 to 3 to 00-, 3 to 200-, 3 to 100-, 3 to 90-, 3 to 80-, 3 to 70-, 3 to 60-, 3 to 50-, 3 to 40-, 3 to 30-, 3 to 20-, 3 to 10-, 3 to 9-, 3 to 8-, 3 to 7-, 3 to 6-, 3 to 5-, 3 to 4-, 4 to 1000-, 4 to 900-, 4 to 800-, 4 to 700-, 4 to 600-, 4 to 500-, 4 to 400-, 4 to 4 to 00-, 4 to 200-, 4 to 100-, 4 to 90-, 4 to 80-, 4 to 70-, 4 to 60-, 4 to 50-, 4 to 40-, 4 to 30-, 4 to 20-, 4 to 10-, 4 to 9-, 4 to 8-, 4 to 7-, 4 to 6-, 4 to 5-, 4 to 4-, 5 to 1000-, 5 to 900-, 5 to 800-, 5 to 700-, 5 to 600-, 5 to 500-, 5 to 400-, 5 to 300-, 5 to 200-, 5 to 100-, 5 to 90-, 5 to 80-, 5 to 70-, 5 to 60-, 5 to 50-, 5 to 40-, 5 to 30-, 5 to 20-, 5 to 10-, 5 to 9-, 5 to 8-, 5 to 7-, 5 to 6-, 6 to 1000-, 6 to 900-, 6 to 800-, 6 to 700-, 6 to 600-, 6 to 500-, 6 to 400-, 6 to 300-, 6 to 200-, 6 to 100-, 6 to 90-, 6 to 80-, 6 to 70-, 6 to 60-, 6 to 50-, 6 to 40-, 6 to 30-, 6 to 20-, 6 to 10-, 6 to 9-, 6 to 8-, 6 to 7-, 7 to 1000-, 7 to 900-,7 to 800-, 7 to 700-, 7 to 600-, 7 to 500-, 7 to 400-, 7 to 300-, 7 to 200-, 7 to 100-, 7 to 90-, 7 to 80-, 7 to 70-, 7 to 60-, 7 to 50-, 7 to 40-, 7 to 30-, 7 to 20-, 7 to 10-, 7 to 9-, 7 to 8-, 8 to 1000-, 8 to 900-, 8 to 800-, 8 to 700-, 8 to 600-, 8 to 500-, 8 to 400-, 8 to 300-, 8 to 200-, 8 to 100-, 8 to 90-, 8 to 80-, 8 to 70-, 8 to 60-, 8 to 50-, 8 to 40-, 8 to 30-, 8 to 20-, 8 to 10-, 8 to 9-, 9 to 1000-, 9 to 900-, 9 to 800-, 9 to 700-, 9 to 600-, 9 to 500-, 9 to 400-, 9 to 300-, 9 to 200-, 9 to 100-, 9 to 90-, 9 to 80-, 9 to 70-, 9 to 60-, 9 to 50-, 9 to 40-, 9 to 30-, 9 to 20-, 9 to 10-, 10 to 1000-, 10 to 900-, 10 to 800-, 10 to 700-, 10 to 600-, 10 to 500-, 10 to 400-, 10 to 300-, 10 to 200-, 10 to 100-, 10 to 90-, 10 to 80-, 10 to 70-, 10 to 60-, 10 to 50-, 10 to 40-, 10 to 30-, 10 to 20-, 20 to 1000-, 20 to 900-, 20 to 800-, 20 to 700-, 20 to 600-, 20 to 500-, 20 to 400-, 20 to 300-, 20 to 200-, 20 to 100-, 20 to 90-, 20 to 80-, 20 to 70-, 20 to 60-, 20 to 50-, 20 to 40-, 20 to 30-, 30 to 1000-, 30 to 900-, 30 to 800-, 30 to 700-, 30 to 600-, 30 to 500-, 30 to 400-, 30 to 300-, 30 to 200-, 30 to 100-, 30 to 90-, 30 to 80-, 30 to 70-, 30 to 60-, 30 to 50-, 30 to 40-, 40 to 1000-, 40 to 900-, 40 to 800-, 40 to 700-, 40 to 600-, 40 to 500-, 40 to 400-, 40 to 300-, 40 to 200-, 40 to 100-, 40 to 90-, 40 to 80-, 40 to 70-, 40 to 60-, 40 to 50-, 50 to 1000-, 50 to 900-, 50 to 800-, 50 to 700-, 50 to 600-, 50 to 500-, 50 to 400-, 50 to 300-, 50 to 200-, 50 to 100-, 50 to 90-, 50 to 80-, 50 to 70-, 50 to 60-, 60 to 1000-, 60 to 900-, 60 to 800-, 60 to 700-, 60 to 600-, 60 to 500-, 60 to 400-, 60 to 300-, 60 to 200-, 60 to 100-, 60 to 90-, 60 to 80-, 60 to 70-, 70 to 1000-, 70 to 900-, 70 to 800-, 70 to 700-, 70 to 600-, 70 to 500-, 70 to 400-, 70 to 300-, 70 to 200-, 70 to 100-, 70 to 90-, 70 to 80-, 80 to 1000-, 80 to 900-, 80 to 800-, 80 to 700-, 80 to 600-, 80 to 500-, 80 to 400-, 80 to 300-, 80 to 200-, 80 to 100-, 80 to 90-, 90 to 1000-, 90 to 900-, 90 to 800-, 90 to 700-, 90 to 600-, 90 to 500-, 90 to 400-, 90 to 300-, 90 to 200-, 90 to 100-, 100 to 1000-, 100 to 900-, 100 to 800-, 100 to 700-, 100 to 600-, 100 to 500-, 100 to 400-, 100 to 300-, 100 to 200-, 200 to 1000-, 200 to 900-, 200 to 800-, 200 to 700-, 200 to 600-, 200 to 500-, 200 to 400-, 200 to 300-, 300 to 1000-, 300 to 900-, 300 to 800-, 300 to 700-, 300 to 600-, 300 to 500-, 300 to 400-, 400 to 1000-, 400 to 900-, 400 to 800-, 400 to 700-, 400 to 600-, 400 to 500-, 500 to 1000-, 500 to 900-, 500 to 800-, 500 to 700-, 500 to 600-, 600 to 1000-, 600 to 900-, 600 to 800-, 600 to 700-, 700 to 1000-, 700 to 900-, 700 to 800-, 800 to 1000-, 800 to 900-, or 900 to 1000-fold reduction in the standard of care dose of a recombinant Ebola virus and/or Marburg virus protein vaccine. In some embodiments, such as the foregoing, the anti-Ebola virus and/or anti-Marburg virus antigenic polypeptide antibody titer produced in the subject is equivalent to an anti-Ebola virus and/or anti-Marburg virus antigenic polypeptide antibody titer produced in a control subject administered the standard of care dose of a recombinant or purified Ebola virus and/or Marburg virus protein vaccine or a live attenuated or inactivated Ebola virus and/or Marburg virus vaccine. In some embodiments, the effective amount is a dose equivalent to (or equivalent to an at least) 2-, 3-, 4-, 5-, 6-, 7-, 8-, 9-, 10-, 20-, 30-, 40-, 50-, 60-, 70-, 80-, 90-, 100-, 110-, 120-, 130-, 140-, 150-, 160-, 170-, 1280-, 190-, 200-, 210-, 220-, 230-, 240-, 250-, 260-, 270-, 280-, 290-, 300-, 310-, 320-, 330-, 340-, 350-, 360-, 370-, 380-, 390-, 400-, 410-, 420-, 430-, 440-, 450-, 4360-, 470-, 480-, 490-, 500-, 510-, 520-, 530-, 540-, 550-, 560-, 5760-, 580-, 590-, 600-, 610-, 620-, 630-, 640-, 650-, 660-, 670-, 680-, 690-, 700-, 710-, 720-, 730-, 740-, 750-, 760-, 770-, 780-, 790-, 800-, 810-, 820-, 830-, 840-, 850-, 860-, 870-, 880-, 890-, 900-, 910-, 920-, 930-, 940-, 950-, 960-, 970-, 980-, 990-, or 1000-fold reduction in the standard of care dose of a recombinant Ebola virus and/or Marburg virus protein vaccine. In some embodiments, such as the foregoing, an anti-Ebola virus and/or anti Marburg virus antigenic polypeptide antibody titer produced in the subject is equivalent to an anti-Ebola virus and/or anti-Marburg virus antigenic polypeptide antibody titer produced in a control subject administered the standard of care dose of a recombinant or purified Ebola virus and/or Marburg virus protein vaccine or a live attenuated or inactivated Ebola virus and/or Marburg virus vaccine.

In some embodiments, the effective amount of a Ebola virus and/or Marburg virus RNA (e.g., mRNA) vaccine is a total dose of 50-1000 μg. In some embodiments, the effective amount of a Ebola virus and/or Marburg virus RNA (e.g., mRNA) vaccine is a total dose of 50-1000, 50-900, 50-800, 50-700, 50-600, 50-500, 50-400, 50-300, 50-200, 50-100, 50-90, 50-80, 50-70, 50-60, 60-1000, 60-900, 60-800, 60-700, 60-600, 60-500, 60-400, 60-300, 60-200, 60-100, 60-90, 60-80, 60-70, 70-1000, 70-900, 70-800, 70-700, 70-600, 70-500, 70-400, 70-300, 70-200, 70-100, 70-90, 70-80, 80-1000, 80-900, 80-800, 80-700, 80-600, 80-500, 80-400, 80-300, 80-200, 80-100, 80-90, 90-1000, 90-900, 90-800, 90-700, 90-600, 90-500, 90-400, 90-300, 90-200, 90-100, 100-1000, 100-900, 100-800, 100-700, 100-600, 100-500, 100-400, 100-300, 100-200, 200-1000, 200-900, 200-800, 200-700, 200-600, 200-500, 200-400, 200-300, 300-1000, 300-900, 300-800, 300-700, 300-600, 300-500, 300-400, 400-1000, 400-900, 400-800, 400-700, 400-600, 400-500, 500-1000, 500-900, 500-800, 500-700, 500-600, 600-1000, 600-900, 600-900, 600-700, 700-1000, 700-900, 700-800, 800-1000, 800-900, or 900-1000 μg. In some embodiments, the effective amount of a Ebola virus and/or Marburg virus RNA (e.g., mRNA) vaccine is a total dose of 50, 100, 150, 200, 250, 300, 350, 400, 450, 500, 550, 600, 650, 700, 750, 800, 850, 900, 950 or 1000 μg. In some embodiments, the effective amount is a dose of 25-500 μg administered to the subject a total of two times. In some embodiments, the effective amount of a Ebola virus and/or Marburg virus RNA (e.g., mRNA) vaccine is a dose of 25-500, 25-400, 25-300, 25-200, 25-100, 25-50, 50-500, 50-400, 50-300, 50-200, 50-100, 100-500, 100-400, 100-300, 100-200, 150-500, 150-400, 150-300, 150-200, 200-500, 200-400, 200-300, 250-500, 250-400, 250-300, 300-500, 300-400, 350-500, 350-400, 400-500 or 450-500 μg administered to the subject a total of two times. In some embodiments, the effective amount of a Ebola virus and/or Marburg virus RNA (e.g., mRNA) vaccine is a total dose of 25, 50, 100, 150, 200, 250, 300, 350, 400, 450, or 500 μg administered to the subject a total of two times.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EXAMPLES

### Example 1: Manufacture of Polynucleotides

According to the present disclosure, the manufacture of polynucleotides and or parts or regions thereof may be accomplished utilizing the methods taught in International Application WO2014/152027 entitled “Manufacturing Methods for Production of RNA Transcripts”, the contents of which is incorporated herein by reference in its entirety.

Purification methods may include those taught in International Application WO2014/152030 and WO2014/152031, each of which is incorporated herein by reference in its entirety.

Detection and characterization methods of the polynucleotides may be performed as taught in WO2014/144039, which is incorporated herein by reference in its entirety.

Characterization of the polynucleotides of the disclosure may be accomplished using a procedure selected from the group consisting of polynucleotide mapping, reverse transcriptase sequencing, charge distribution analysis, and detection of RNA impurities, wherein characterizing comprises determining the RNA transcript sequence, determining the purity of the RNA transcript, or determining the charge heterogeneity of the RNA transcript. Such methods are taught in, for example, WO2014/144711 and WO2014/144767, the contents of each of which is incorporated herein by reference in its entirety.

### Example 2: Chimeric Polynucleotide Synthesis

### Introduction

According to the present disclosure, two regions or parts of a chimeric polynucleotide may be joined or ligated using triphosphate chemistry.

According to this method, a first region or part of 100 nucleotides or less is chemically synthesized with a 5′ monophosphate and terminal 3′desOH or blocked OH. If the region is longer than 80 nucleotides, it may be synthesized as two strands for ligation.

If the first region or part is synthesized as a non-positionally modified region or part using in vitro transcription (IVT), conversion the 5′monophosphate with subsequent capping of the 3′ terminus may follow.

Monophosphate protecting groups may be selected from any of those known in the art.

The second region or part of the chimeric polynucleotide may be synthesized using either chemical synthesis or IVT methods. IVT methods may include an RNA polymerase that can utilize a primer with a modified cap. Alternatively, a cap of up to 130 nucleotides may be chemically synthesized and coupled to the IVT region or part.

It is noted that for ligation methods, ligation with DNA T4 ligase, followed by treatment with DNAse should readily avoid concatenation.

The entire chimeric polynucleotide need not be manufactured with a phosphate-sugar backbone. If one of the regions or parts encodes a polypeptide, then it is preferable that such region or part comprise a phosphate-sugar backbone.

Ligation is then performed using any known click chemistry, orthoclick chemistry, solulink, or other bioconjugate chemistries known to those in the art.

### Synthetic Route

The chimeric polynucleotide is made using a series of starting segments. Such segments include:

(a) Capped and protected 5′ segment comprising a normal 3′OH (SEG. 1)

(b) 5′ triphosphate segment which may include the coding region of a polypeptide and comprising a normal 3′OH (SEG. 2)

(c) 5′ monophosphate segment for the 3′ end of the chimeric polynucleotide (e.g., the tail) comprising cordycepin or no 3′OH (SEG. 3)

After synthesis (chemical or IVT), segment 3 (SEG. 3) is treated with cordycepin and then with pyrophosphatase to create the 5′monophosphate.

Segment 2 (SEG. 2) is then ligated to SEG. 3 using RNA ligase. The ligated polynucleotide is then purified and treated with pyrophosphatase to cleave the diphosphate. The treated SEG.2-SEG. 3 construct is then purified and SEG. 1 is ligated to the 5′ terminus. A further purification step of the chimeric polynucleotide may be performed.

Where the chimeric polynucleotide encodes a polypeptide, the ligated or joined segments may be represented as: 5′UTR (SEG. 1), open reading frame or ORF (SEG. 2) and 3′UTR+PolyA (SEG. 3).

The yields of each step may be as much as 90-95%.

### Example 3: PCR for cDNA Production

PCR procedures for the preparation of cDNA are performed using 2×KAPA HIFI™ HotStart ReadyMix by Kapa Biosystems (Woburn, Mass.). This system includes 2×KAPA ReadyMix12.5 μl; Forward Primer (10 μM) 0.75 μl; Reverse Primer (10 μM) 0.75 μl; Template cDNA −100 ng; and dH₂O diluted to 25.0 μl. The reaction conditions are at 95° C. for 5 min. and 25 cycles of 98° C. for 20 sec, then 58° C. for 15 sec, then 72° C. for 45 sec, then 72° C. for 5 min. then 4° C. to termination.

The reaction is cleaned up using Invitrogen's PURELINK™ PCR Micro Kit (Carlsbad, Calif.) per manufacturer's instructions (up to 5 μg). Larger reactions will require a cleanup using a product with a larger capacity. Following the cleanup, the cDNA is quantified using the NANODROP™ and analyzed by agarose gel electrophoresis to confirm the cDNA is the expected size. The cDNA is then submitted for sequencing analysis before proceeding to the in vitro transcription reaction.

### Example 4: In Vitro Transcription (IVT)

The in vitro transcription reaction generates polynucleotides containing uniformly modified polynucleotides. Such uniformly modified polynucleotides may comprise a region or part of the polynucleotides of the disclosure. The input nucleotide triphosphate (NTP) mix is made in-house using natural and un-natural NTPs.

A typical in vitro transcription reaction includes the following:

| | | | |
|---|---|---|---|
| 1 | Template cDNA | 1.0 | μg |
| 2 | 10x transcription buffer (400 mM Tris-HCl pH | 2.0 | μl |
| | 8.0, 190 mM MgCl₂, 50 mM DTT, 10 mM | | |
| | Spermidine) | | |
| 3 | Custom NTPs (25 mM each) | 7.2 | μl |
| 4 | RNase Inhibitor | 20 | U |
| 5 | T7 RNA polymerase | 3000 | U |

| | | |
|---|---|---|
| 6 | dH₂0 | Up to 20.0 μl. and |
| 7 | Incubation at 37° C. for 3 hr-5 hrs. | |

The crude IVT mix may be stored at 4° C. overnight for cleanup the next day. 1 U of RNase-free DNase is then used to digest the original template. After 15 minutes of incubation at 37° C., the mRNA is purified using Ambion's MEGACLEAR™ Kit (Austin, Tex.) following the manufacturer's instructions. This kit can purify up to 500 μg of RNA. Following the cleanup, the RNA is quantified using the NanoDrop and analyzed by agarose gel electrophoresis to confirm the RNA is the proper size and that no degradation of the RNA has occurred.

### Example 5: Enzymatic Capping

Capping of a polynucleotide is performed as follows where the mixture includes: IVT RNA 60 μg-180 μg and dH₂O up to 72 μl. The mixture is incubated at 65° C. for 5 minutes to denature RNA, and then is transferred immediately to ice.

The protocol then involves the mixing of 10× Capping Buffer (0.5 M Tris-HCl (pH 8.0), 60 mM KCl, 12.5 mM MgCl₂) (10.0 μl); 20 mM GTP (5.0 μl); 20 mM S-Adenosyl Methionine (2.5 μl); RNase Inhibitor (100 U); 2′-O-Methyltransferase (400U); Vaccinia capping enzyme (Guanylyl transferase) (40 U); dH₂O (Up to 28 μl); and incubation at 37° C. for 30 minutes for 60 μg RNA or up to 2 hours for 180 μg of RNA.

The polynucleotide is then purified using Ambion's MEGACLEAR™ Kit (Austin, Tex.) following the manufacturer's instructions. Following the cleanup, the RNA is quantified using the NANODROP™ (ThermoFisher, Waltham, Mass.) and analyzed by agarose gel electrophoresis to confirm the RNA is the proper size and that no degradation of the RNA has occurred. The RNA product may also be sequenced by running a reverse-transcription-PCR to generate the cDNA for sequencing.

### Example 6: PolyA Tailing Reaction

Without a poly-T in the cDNA, a poly-A tailing reaction must be performed before cleaning the final product. This is done by mixing Capped IVT RNA (100 μl); RNase Inhibitor (20 U); 10× Tailing Buffer (0.5 M Tris-HCl (pH 8.0), 2.5 M NaCl, 100 mM MgCl₂)(12.0 μl); 20 mM ATP (6.0 μl); Poly-A Polymerase (20 U); dH₂O up to 123.5 μl and incubation at 37° C. for 30 min. If the poly-A tail is already in the transcript, then the tailing reaction may be skipped and proceed directly to cleanup with Ambion's MEGACLEAR™ kit (Austin, Tex.) (up to 500 μg). Poly-A Polymerase is preferably a recombinant enzyme expressed in yeast.

It should be understood that the processivity or integrity of the polyA tailing reaction may not always result in an exact size polyA tail. Hence polyA tails of approximately between 40-200 nucleotides, e.g., about 40, 50, 60, 70, 80, 90, 91, 92, 93, 94, 95, 96, 97, 98, 99, 100, 101, 102, 103, 104, 105, 106, 107, 108, 109, 110, 150-165, 155, 156, 157, 158, 159, 160, 161, 162, 163, 164 or 165 are within the scope of the invention.

### Example 7: Natural 5′ Caps and 5′ Cap Analogues

5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap];G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes are preferably derived from a recombinant source.

When transfected into mammalian cells, the modified mRNAs have a stability of between 12-18 hours or more than 18 hours, e.g., 24, 36, 48, 60, 72 or greater than 72 hours.

### Example 8: Capping Assays

A. Protein Expression Assay

Polynucleotides encoding a polypeptide, containing any of the caps taught herein can be transfected into cells at equal concentrations. 6, 12, 24 and 36 hours post-transfection the amount of protein secreted into the culture medium can be assayed by ELISA. Synthetic polynucleotides that secrete higher levels of protein into the medium would correspond to a synthetic polynucleotide with a higher translationally-competent Cap structure.

B. Purity Analysis Synthesis

Polynucleotides encoding a polypeptide, containing any of the caps taught herein can be compared for purity using denaturing Agarose-Urea gel electrophoresis or HPLC analysis.

Polynucleotides with a single, consolidated band by electrophoresis correspond to the higher purity product compared to polynucleotides with multiple bands or streaking bands. Synthetic polynucleotides with a single HPLC peak would also correspond to a higher purity product. The capping reaction with a higher efficiency would provide a more pure polynucleotide population.

C. Cytokine Analysis

Polynucleotides encoding a polypeptide, containing any of the caps taught herein can be transfected into cells at multiple concentrations. 6, 12, 24 and 36 hours post-transfection the amount of pro-inflammatory cytokines such as TNF-alpha and IFN-beta secreted into the culture medium can be assayed by ELISA. Polynucleotides resulting in the secretion of higher levels of pro-inflammatory cytokines into the medium would correspond to a polynucleotides containing an immune-activating cap structure.

D. Capping Reaction Efficiency

Polynucleotides encoding a polypeptide, containing any of the caps taught herein can be analyzed for capping reaction efficiency by LC-MS after nuclease treatment. Nuclease treatment of capped polynucleotides would yield a mixture of free nucleotides and the capped 5′-5-triphosphate cap structure detectable by LC-MS. The amount of capped product on the LC-MS spectra can be expressed as a percent of total polynucleotide from the reaction and would correspond to capping reaction efficiency. The cap structure with higher capping reaction efficiency would have a higher amount of capped product by LC-MS.

### Example 9: Agarose Gel Electrophoresis of Modified RNA or RT PCR Products

Individual polynucleotides (200-400 ng in a 20 μl volume) or reverse transcribed PCR products (200-400 ng) are loaded into a well on a non-denaturing 1.2% Agarose E-Gel (Invitrogen, Carlsbad, Calif.) and run for 12-15 minutes according to the manufacturer protocol.

### Example 10: Nanodrop Modified RNA Quantification and UV Spectral Data

Modified polynucleotides in TE buffer (1 μl) are used for Nanodrop UV absorbance readings to quantitate the yield of each polynucleotide from a chemical synthesis or in vitro transcription reaction.

### Example 11: Formulation of Modified mRNA Using Lipidoids

Polynucleotides are formulated for in vitro experiments by mixing the polynucleotides with the lipidoid at a set ratio prior to addition to cells. In vivo formulation may require the addition of extra ingredients to facilitate circulation throughout the body. To test the ability of these lipidoids to form particles suitable for in vivo work, a standard formulation process used for siRNA-lipidoid formulations may used as a starting point. After formation of the particle, polynucleotide is added and allowed to integrate with the complex. The encapsulation efficiency is determined using a standard dye exclusion assays.

### Example 12: Evaluation of Immunogenicity of Ebola GP Antigens

### Introduction

The Ebola glycoprotein (GP) is the only virally expressed protein on the virion surface, where it is essential for the attachment to host cells and catalyzes membrane fusion. Therefore, the Ebola GP is a critical component of vaccines, as well as a target of neutralizing antibodies and inhibitors of attachment and fusion. Pre-GP is cleaved by furin at a multi-basic motif into two subunits, GP1 and GP2, which remain associated through a disulfide linkage between Cys53 of GP1 and Cys609 of GP2. The heterodimer (GP1 and GP2) then assembles into a 450-kDa trimer (3 GP1 and 3 GP2) at the surface of nascent virions, where it exerts its functions. The structure of Ebola GP and antigen constructs tested herein are shown in FIG. 1 and Table 1.

Briefly, “Matured” EBOV GP has been engineered to include a human signal peptide. “Secreted” EBOV GP has been engineered to remove the transmembrane domain, i.e., residues 651-676 as depicted in the schematic. The “peptide scaffold” is as described in Schroder et al 2008. This short peptide sequence is described in the art as being capable of facilitating nanostructure formation. In preliminary experiments, the scaffold did not enhance antigenicity in the constructs tested. Without being bound in theory, it is believed that the scaffold peptide is not able to facilitate nanostructure formation under the physiologic conditions exemplified.

TABLE 1
Ebola glycoprotein constructs

| Antigen | Description | Cellular localization |
|---|---|---|
| Wildtype GP pro- | Canonical Ebola Zaire AA sequence of pro-polypeptide | Surface, membrane |
| polypeptide (surface form) | of GP, which is then processed post translationally. | bound |
| Wildtype GP pro- | Canonical Ebola Zaire AA sequence of pro-polypeptide | Surface, membrane |
| polypeptide (surface form, | of GP, which is then processed post translationally, with | bound |
| v5 tagged) | a V5 tag | |
| Matured GP polypeptide | Containing the AA sequence of the post-translationally | Surface, membrane |
| (Surface form) | processed GP. | bound |
| Matured GP polypeptide | Containing the AA sequence of the post-translationally | Surface, membrane |
| (Surface form, v5 tagged) | processed GP, with a V5 tag | bound |
| Wildtype GP pro- | Canonical Ebola Zaire AA sequence of pro-polypeptide | Secreted |
| polypeptide (secreted | of GP without the transmembrane domain, which is then | |
| form) | processed post translationally, | |
| Matured GP polypeptide | Containing the AA sequence of the post-translationally | secreted |
| (secreted form) | processed GP, minus the transmembrane domain | |
| Matured GP polypeptide | Containing the AA sequence of the post-translationally | secreted |
| in scaffold (secreted form) | processed GP, minus the transmembrane domain, with a | |
| | self-assembly peptidic scaffold | |

### Immunogenicity Evaluation: Study Design

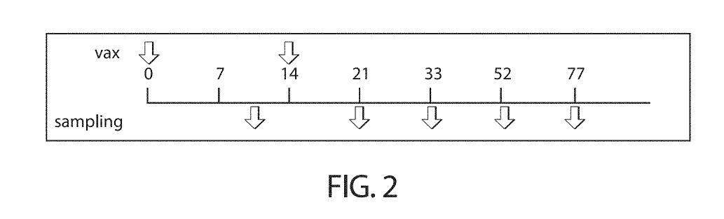

In order to evaluate the antigenicity of seven Ebola antigen constructs, the following protocol was developed. As shown in FIG. 2, the mice were vaccinated with MC3-formulated, mRNA-encoded Ebola GP on days 0 (primary) and 14 (first booster). The doses were 0.4 mg/kg. Samples were collected on days 0, 10, 21, 33, 52, and 77. Mice were euthanized on Day 77. Recombinant EBOV GP and PBS were used as the positive and negative experimental controls, respectively.

### Results

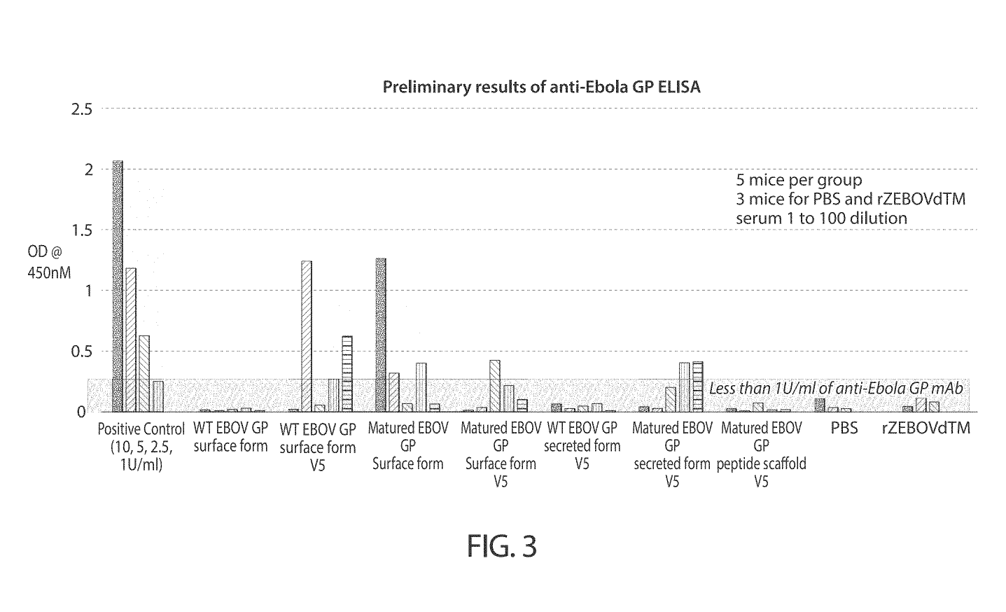

The initial anti-Ebola GP response at Day 10 after a single primary challenge was generally within the range of 1U/mL of anti-Ebola GP mouse antibody, as measured by ELISA (FIG. 3). Serum samples were diluted 1:100 for the assay. For the positive control, the colored bars represent the units depicted. For the various constructs tested, the colored bars represent antibody titers for individual mice tested. As compared to PBS control, essentially all constructs had detectable antibody titers at 10 days following immunization.

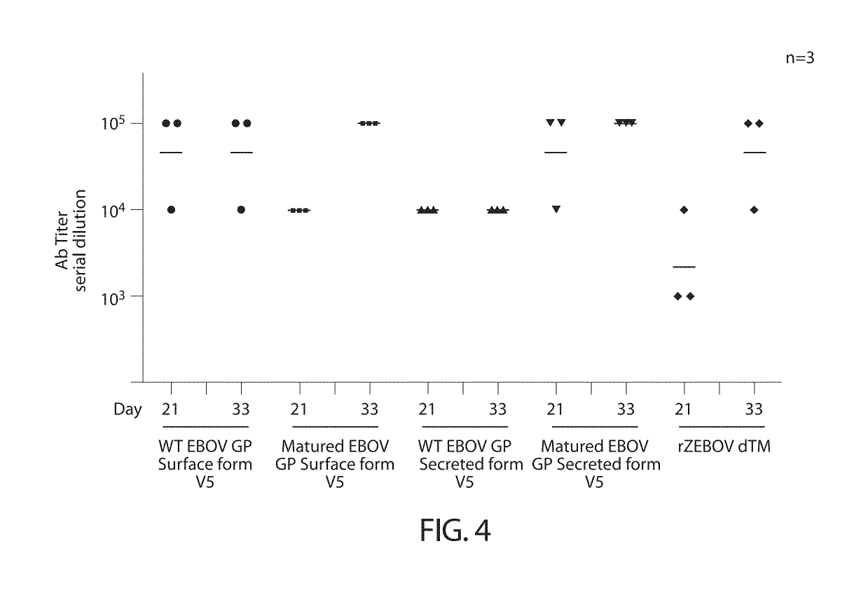

The anti-Ebola GP antibody titer of selected antigens on Day 21 and Day 23 was also examined (n=3 per group) (FIG. 4).

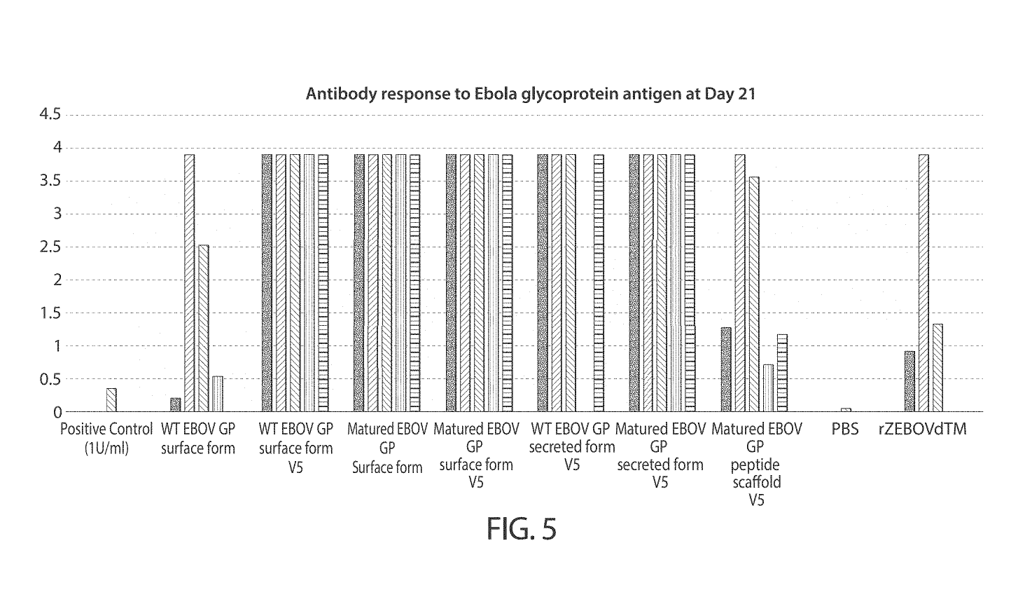

The antibody response to the Ebola GP antigen at Day 21 post-vaccination was also quantified in each group (FIG. 5). For the positive control, the colored bars represent the units depicted. For the various constructs tested, the colored bars represent antibody titers for individual mice tested. As compared to PBS control, essentially all constructs had significant antibody titers at 10 days following immunization.

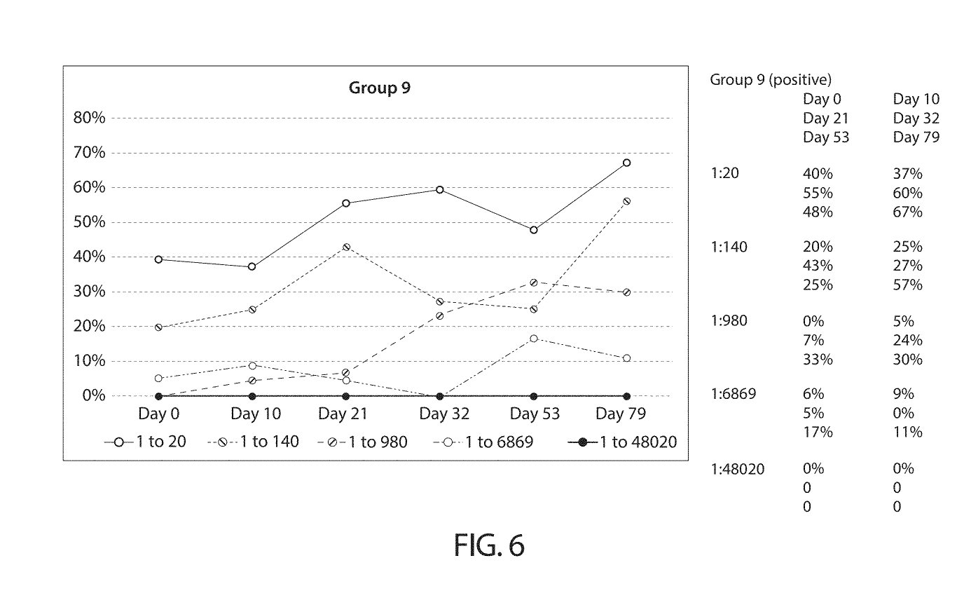

The in vitro neutralization activity of serum samples in the DeltaVp30 Ebola virus system has been examined (Halfman et al., PNAS 105:1129) (FIG. 6). Naïve mouse serum was found to have blocking activity in the assay, which was particularly evident at the 1:20 dilution and is believed to have masked the potential specific neutralizing activity of serum samples from animals at higher concentrations. The 1:980 dilution was found to be the best with respect to the evaluating the virus neutralizing capability of the samples. The background at this dilution was typically less than 10% on the Day 0 time point.

### Example 13: In Vivo Vaccination of Guinea Pigs

### Introduction

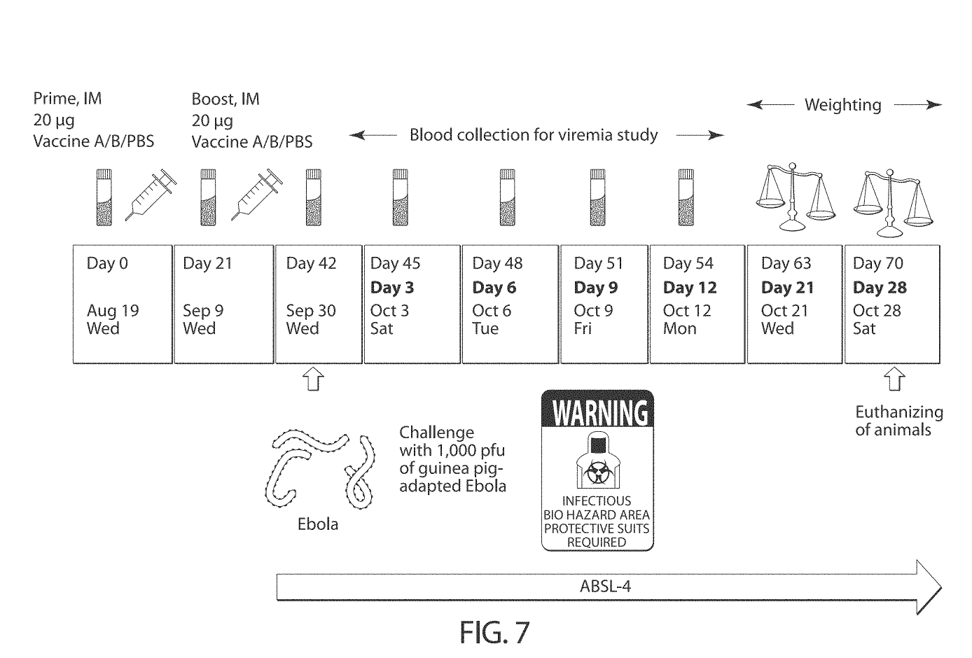

Guinea Pigs were vaccinated with Ebola GP (either pre-protein GP or mature GP) according to the vaccination schedule shown in FIG. 7 and Table 2.

TABLE 2

| | N | | | |
|---|---|---|---|---|
| Group | animals | Vaccine | Dose | Route |
| A | 5 | EBOV GP (pre-protein) | 20 ug/100 ul | IM |
| | | membrane | | |
| B | 5 | EBOV mature GP (IgK - | 20 ug/100 ul | IM |
| | | membrane bound) | | |
| C | 5 | PBS | 100 ul | IM |

The guinea pigs were primed with 20 ug of vaccine on day 0 and boosted with 20 ug of vaccine on day 21 (both IM). Animals were challenged with 1,000 pfu of guinea pig-adapted Ebola virus on day 42. Blood was collected on days 42, 45, 48, 51, 54, 63, and 70, followed by euthanization of the animals on day 70. Two mRNA vaccine constructs were tested (EH_EBLA.matGP.IgKsp(mem) SEQ ID NO. 17 and EH_EBLA.wtGP(mem) SEQ ID NO. 21) and a control unvaccinated group received PBS. Dosing is IM and there are 2 doses/animal (2 and 10 ug). The mRNA vaccines include pseudo uridine modifications.

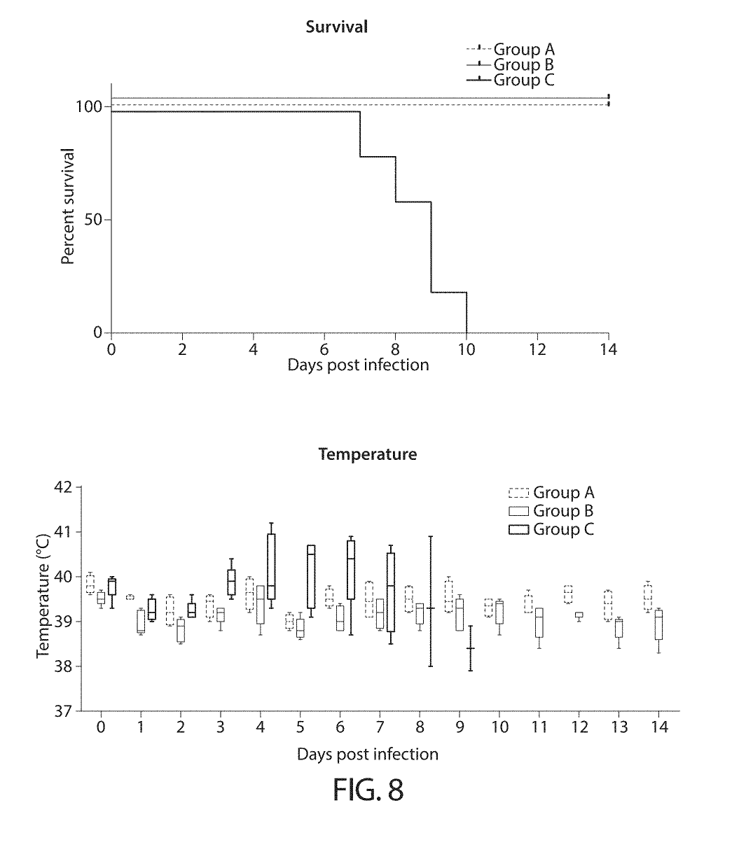

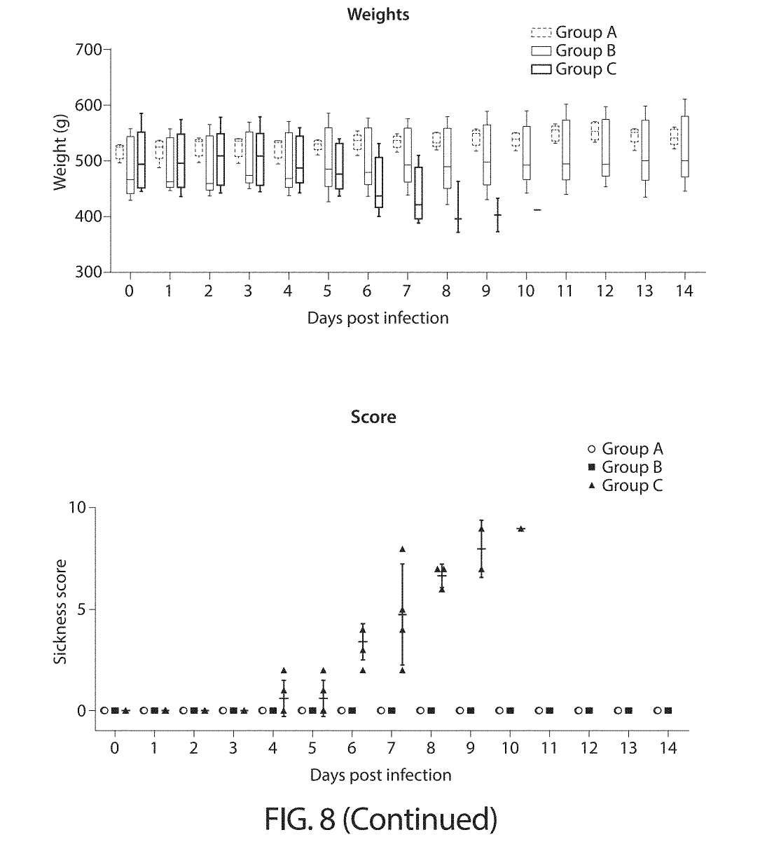

Quite surprisingly, vaccination with the mRNA vaccine conferred 100% protection against 10E3 PFUs of gp-adapted Ebola (Zaire species, Mayinga strain). Untreated animals succumbed to the infection completely by day 10 post infection. The data is shown in FIG. 8.

### Example 14: In Vivo Immunogenicity of BALB/c Mice

Marburg glycoprotein mRNA vaccines were generated using three distinct Marburg strains based on glycoprotein sequence conservation: Musoke, Uganda, and RAVN. The mRNA constructs were tested for in vitro expression by transfection in mammalian cells and protein detection using Western blot (data not shown).

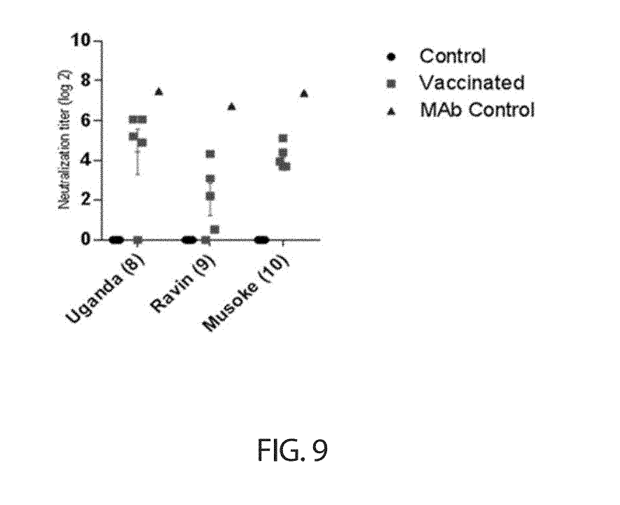

To test in vivo immunogenicity, BALB/c mice were administered 10 μg of the vaccine on day 0 and day 28. Neutralizing titers to their homologous strains were measured on day 56. As shown in FIG. 9, all mRNA glycoprotein vaccines tested in this study were found to be immunogenic with high levels of neutralizing titers against their specific strains including Uganda (group 8), Ravin (group 9), and Musoke (group 10).

### Sequences

The following are Ebola nucleic acid sequences (Table 4) for the open reading frames of the RNA polynucleotides (Table 6) or for the RNA polynucleotides and amino acid (Table 5) sequences for each of the exemplary constructs. Tables 7 and 8 provide the DNA and RNA sequences of the full constructs, respectively. With respect to the Marburg virus sequences, Table 10 provides amino acid the amino acid sequences, first with the signal sequence and then without the signal sequence, Table 11 provides DNA sequences, and Table 12 shows the RNA polynucleotides.

It should be understood that any of the mRNA sequences described herein may include a 5′ UTR and/or a 3′ UTR. The UTR sequences may be selected from the following sequences, or other known UTR sequences may be used. It should also be understood that any of the mRNA constructs described herein may further comprise a polyA tail and/or cap (e.g., 7mG(5′)ppp(5′)NlmpNp). Further, while many of the mRNAs and encoded antigen sequences described herein include a signal peptide and/or a peptide tag (e.g., C-terminal His tag), it should be understood that the indicated signal peptide and/or peptide tag may be substituted for a different signal peptide and/or peptide tag, or the signal peptide and/or peptide tag may be omitted.

DNA
5′ UTR:
(SEQ ID NO: 142)
tcaagctttt ggaccctcgt acagaagcta atacgactca
ctatagggaa ataagagaga aaagaagagt aagaagaaat
ataagagcca cc
5′ UTR:
(SEQ ID NO: 144)
gggaaataag agagaaaaga agagtaagaa gaaatataag
accccggcgc cgccacc
3′ UTR:
(SEQ ID NO: 143)
tgataatagg ctggagcctc ggtggccatg cttcttgccc
cttgggcctc cccccagccc ctcctcccct tcctgcaccc
gtacccccgt ggtctttgaa taaagtctga gtgggcggc
RNA
5′ UTR:
(SEQ ID NO: 163)
ucaagcuuuu ggacccucgu acagaagcua auacgacuca
cuauagggaa auaagagaga aaagaagagu aagaagaaau
auaagagcca cc
5′ UTR:
(SEQ ID NO: 165)
gggaaauaag agagaaaaga agaguaagaa gaaauauaag
accccggcgc cgccacc
3′ UTR:
(SEQ ID NO: 164)
ugauaauagg cuggagccuc gguggccaug cuucuugccc
cuugggccuc cccccagccc cuccuccccu uccugcaccc
guacccccgu ggucuuugaa uaaagucuga gugggcggc

Each of the sequences described herein encompasses a chemically modified sequence (one or more nucleotides are modified) or an unmodified sequence which includes no nucleotide modifications.

TABLE 3

| | Nucleic | Amino | Nucleic |
|---|---|---|---|
| | Acid | Acid | Acid poly- |
| | ORF | ORF | nucleotide |
| | SEQ ID | SEQ ID | SEQ ID |
| mRNA Name(s) | NO | NO | NO |
| EBLA.matureGP.IgKsp(surface) | 1 | 9 | 17 |
| EBLA.matureGP.IgKsp(surface).v5 | 2 | 10 | 18 |
| EBLA.matureGP.IgKsp(Secreted) | 3 | 11 | 19 |
| EBLA.matureGP.IgKsp(Secreted).v5 | 4 | 12 | 20 |
| EBLA.wtGP(surface) | 5 | 13 | 21 |
| EBLA.wtGP(surface).v5 | 6 | 14 | 22 |
| EBLA.wtGP(secreted) | 7 | 15 | 23 |
| EBLA.wtGP(secreted).v5 | 8 | 16 | 24 |
| EBLA.wtNP | 25 | 26 | 27 |
| GP12_SUDN_609_2012 | 45 | 46/47 | |
| GP12_ZEBOV_GBR_15 | 49 | 50/51 | |
| GP12_Bundi_112_2012 | 53 | 54/55 | |

TABLE 4
EBOV DNA ORF Sequences

| SEQ | |
|---|---|
| ID | |
| NO. | Description |
| 1 | EBLA.matureGP.IgKsp(surface) |
| 2 | EBLA.matureGP.IgKsp(surface).v5 |
| 3 | EBLA.matureGP.IgKsp(Secreted) |
| 4 | EBLA.wtGP(surface) |
| 5 | EBLA.wtGP(surface).v5 |
| 6 | EBLA.wtGP(secreted) |
| 7 | EBLA.wtGP(secreted).v5 |
| 8 | EBLA.wtNP |
| 25 | GP12_SUDN_609_2012 |
| 45 | GP12_ZEBOV_GBR_15 |
| 49 | GP12_Bundi_112_2012 |
| 53 | EBLA.wtGP(surface) |

TABLE 5
EBOV Protein Sequences

| SEQ | |
|---|---|
| ID | |
| NO. | Description |
| 9 | EBLA.matureGP.IgKsp(surface) |
| 10 | EBLA.matureGP.IgKsp(surface).v5 |
| 11 | EBLA.matureGP.IgKsp(Secreted) |
| 12 | EBLA.wtGP(surface) |
| 13 | EBLA.wtGP(surface).v5 |
| 14 | EBLA.wtGP(secreted) |
| 15 | EBLA.wtGP(secreted).v5 |
| 16 | EBLA.wtNP |
| 26 | GP12_SUDN_609_2012 |
| 46/47 | GP12_ZEBOV_GBR_15 |
| 50/51 | GP12_Bundi_112_2012 |
| 54/55 | EBLA.wtGP(surface) |

TABLE 6
EBOV RNA ORF Sequences

| SEQ | |
|---|---|
| ID | |
| NO. | Description |
| 28 | EBLA.matureGP.IgKsp(surface) |
| 29 | EBLA.matureGP.IgKsp(surface).v5 |
| 30 | EBLA.matureGP.IgKsp(Secreted) |
| 31 | EBLA.wtGP(surface) |
| 32 | EBLA.wtGP(surface).v5 |
| 33 | EBLA.wtGP(secreted) |
| 34 | EBLA.wtGP(secreted).v5 |
| 35 | EBLA.wtNP |
| 141 | GP12_SUDN_609_2012 |
| 48 | GP12_ZEBOV_GBR_15 |
| 52 | GP12_Bundi_112_2012 |
| 56 | EBLA.wtGP(surface) |

TABLE 7
EBOV DNA Constructs (Full Sequences)

| | | 5′ | | 3′ |
|---|---|---|---|---|
| SEQ | | UTR | ORF | UTR |
| ID | | (SEQ | (SEQ | (SEQ |
| NO. | Description | ID NO.) | ID NO.) | ID NO.) |
| 17 | EBLA.matureGP.IgKsp(surface) | 142 | 1 | 143 |
| 18 | EBLA.matureGP.IgKsp(surface).v5 | 142 | 2 | 143 |
| 19 | EBLA.matureGP.IgKsp(Secreted) | 142 | 3 | 143 |
| 20 | EBLA.wtGP(surface) | 142 | 4 | 143 |
| 21 | EBLA.wtGP(surface).v5 | 142 | 5 | 143 |
| 22 | EBLA.wtGP(secreted) | 142 | 6 | 143 |
| 23 | EBLA.wtGP(secreted).v5 | 142 | 7 | 143 |
| 24 | EBLA.wtNP | 142 | 8 | 143 |
| 27 | GP12_SUDN_609_2012 | 142 | 25 | 143 |
| 145 | EBLA.matureGP.IgKsp(surface) | 144 | 1 | 143 |
| 146 | EBLA.matureGP.IgKsp(surface).v5 | 144 | 2 | 143 |
| 147 | EBLA.matureGP.IgKsp(Secreted) | 144 | 3 | 143 |
| 148 | EBLA.wtGP(surface) | 144 | 4 | 143 |
| 149 | EBLA.wtGP(surface).v5 | 144 | 5 | 143 |
| 150 | EBLA.wtGP(secreted) | 144 | 6 | 143 |
| 151 | EBLA.wtGP(secreted).v5 | 144 | 7 | 143 |
| 152 | EBLA.wtNP | 144 | 8 | 143 |
| 153 | GP12_SUDN_609_2012 | 144 | 25 | 143 |

TABLE 8
EBOV RNA Constructs (Full Sequences)

| | | 5′ | | 3′ |
|---|---|---|---|---|
| SEQ | | UTR | ORF | UTR |
| ID | | (SEQ | (SEQ | (SEQ |
| NO. | Description | ID NO.) | ID NO.) | ID NO.) |
| 36 | EBLA.matureGP.IgKsp(surface) | 163 | 28 | 164 |
| 37 | EBLA.matureGP.IgKsp(surface).v5 | 163 | 29 | 164 |
| 38 | EBLA.matureGP.IgKsp(Secreted) | 163 | 30 | 164 |
| 39 | EBLA.wtGP(surface) | 163 | 31 | 164 |
| 40 | EBLA.wtGP(surface).v5 | 163 | 32 | 164 |
| 41 | EBLA.wtGP(secreted) | 163 | 33 | 164 |
| 42 | EBLA.wtGP(secreted).v5 | 163 | 34 | 164 |
| 43 | EBLA.wtNP | 163 | 35 | 164 |
| 44 | GP12_SUDN_609_2012 | 163 | 141 | 164 |
| 154 | EBLA.matureGP.IgKsp(surface) | 165 | 28 | 164 |
| 155 | EBLA.matureGP.IgKsp(surface).v5 | 165 | 29 | 164 |
| 156 | EBLA.matureGP.IgKsp(Secreted) | 165 | 30 | 164 |
| 157 | EBLA.wtGP(surface) | 165 | 31 | 164 |
| 158 | EBLA.wtGP(surface).v5 | 165 | 32 | 164 |
| 159 | EBLA.wtGP(secreted) | 165 | 33 | 164 |
| 160 | EBLA.wtGP(secreted).v5 | 165 | 34 | 164 |
| 161 | EBLA.wtNP | 165 | 35 | 164 |
| 162 | GP12_SUDN_609_2012 | 165 | 141 | 164 |

TABLE 9
Full-length Ebola GP-Small Amino Acid Sequences (Homo sapiens strains)

| GenBank | | Collection | Release | |
|---|---|---|---|---|
| Accession | Country | Date | Date | Virus Name |
| ACI28623 | Uganda | 2007 November | Nov. 21, 2008 | Bundibugyo ebolavirus, complete genome |
| ACI28633 | Cote | 1994 November | Nov. 21, 2008 | Cote d″Ivoire ebolavirus, complete genome |
| | d'Ivoire | | | |
| AFP28230 | Uganda | 2011 May | Aug. 6, 2012 | Sudan ebolavirus - Nakisamata, complete genome |
| ABY75324 | Sudan | 2004 | Jan. 23, 2008 | Sudan ebolavirus isolate EBOV-S-2004 from |
| | | | | Sudan, complete genome |
| AGB56679 | Sudan | 1979 | Jan. 7, 2013 | Sudan ebolavirus isolate SUDV/H.sapiens- |
| | | | | tc/SSD/1979/Maleo, complete genome |
| AER59711 | Democratic | Dec. 31, 2008 | Nov. 7, 2011 | Zaire ebolavirus isolate 034-KS, partial genome |
| | Republic of | | | |
| | the Congo | | | |
| AKU75160 | Sierra | Feb. 19, 2015 | Aug. 11, 2015 | Zaire ebolavirus isolate |
| | Leone | | | EBOV/DML12033/SLe/WesternUrban/20150219, |
| | | | | complete genome |
| AKU75169 | Sierra | Feb. 21, 2015 | Aug. 11, 2015 | Zaire ebolavirus isolate |
| | Leone | | | EBOV/DML12051/SLe/WesternUrban/20150221, |
| | | | | complete genome |
| AKU75178 | Sierra | Feb. 26, 2015 | Aug. 11, 2015 | Zaire ebolavirus isolate |
| | Leone | | | EBOV/DML12116/SLe/WesternUrban/20150226, |
| | | | | complete genome |
| AKU75187 | Sierra | Feb. 26, 2015 | Aug. 11, 2015 | Zaire ebolavirus isolate |
| | Leone | | | EBOV/DML12117/SLe/WesternUrban/20150226, |
| | | | | complete genome |
| AKU75196 | Sierra | Feb. 27, 2015 | Aug. 11, 2015 | Zaire ebolavirus isolate |
| | Leone | | | EBOV/DML12120/SLe/WesternUrban/20150227, |
| | | | | complete genome |
| AKU75214 | Sierra | Feb. 28, 2015 | Aug. 11, 2015 | Zaire ebolavirus isolate |
| | Leone | | | EBOV/DML12137/SLe/WesternUrban/20150228, |
| | | | | complete genome |
| AKU75205 | Sierra | Mar. 4, 2015 | Aug. 11, 2015 | Zaire ebolavirus isolate |
| | Leone | | | EBOV/DML12194/SLe/WesternUrban/20150304, |
| | | | | complete genome |
| AKU75223 | Sierra | Mar. 7, 2015 | Aug. 11, 2015 | Zaire ebolavirus isolate |
| | Leone | | | EBOV/DML12239/SLe/WesternUrban/20150309, |
| | | | | complete genome |
| AKU75232 | Sierra | Mar. 9, 2015 | Aug. 11, 2015 | Zaire ebolavirus isolate |
| | Leone | | | EBOV/DML12260/SLe/WesternUrban/20150309, |
| | | | | complete genome |
| AKU75241 | Sierra | Mar. 10, 2015 | Aug. 11, 2015 | Zaire ebolavirus isolate |
| | Leone | | | EBOV/DML12268/SLe/WesternUrban/20150310, |
| | | | | complete genome |
| AKU75250 | Sierra | Mar. 28, 2015 | Aug. 11, 2015 | Zaire ebolavirus isolate |
| | Leone | | | EBOV/DML12458/SLe/WesternUrban/20150328, |
| | | | | complete genome |
| AKU75259 | Sierra | Mar. 31, 2015 | Aug. 11, 2015 | Zaire ebolavirus isolate |
| | Leone | | | EBOV/DML12485/SLe/WesternUrban/20150331, |
| | | | | partial genome |
| AKU75583 | Sierra | Jun. 30, 2015 | Aug. 11, 2015 | Zaire ebolavirus isolate |
| | Leone | | | EBOV/DML14077/SLe/WesternUrban/20150630, |
| | | | | complete genome |
| AKU75565 | Sierra | Jul. 3, 2015 | Aug. 11, 2015 | Zaire ebolavirus isolate |
| | Leone | | | EBOV/DML14163/SLe/WesternUrban/20150703, |
| | | | | complete genome |
| AKU75574 | Sierra | Jul. 11, 2015 | Aug. 11, 2015 | Zaire ebolavirus isolate |
| | Leone | | | EBOV/DML14366/SLe/WesternUrban/20150711, |
| | | | | complete genome |
| AKU75268 | Sierra | Jan. 13, 2015 | Aug. 11, 2015 | Zaire ebolavirus isolate |
| | Leone | | | EBOV/DML24502/SLe/Kono/20150113, partial |
| | | | | genome |
| AKU75277 | Sierra | Jan. 13, 2015 | Aug. 11, 2015 | Zaire ebolavirus isolate |
| | Leone | | | EBOV/DML24504/SLe/Kono/20150113, |
| | | | | complete genome |
| AKU75286 | Sierra | Jan. 14, 2015 | Aug. 11, 2015 | Zaire ebolavirus isolate |
| | Leone | | | EBOV/DML24506/SLe/Kono/20150114, |
| | | | | complete genome |
| AKU75295 | Sierra | Jan. 14, 2015 | Aug. 11, 2015 | Zaire ebolavirus isolate |
| | Leone | | | EBOV/DML24511/SLe/Kono/20150114, |
| | | | | complete genome |
| AKU75304 | Sierra | Jan. 17, 2015 | Aug. 11, 2015 | Zaire ebolavirus isolate |
| | Leone | | | EBOV/DML24552/SLe/Kono/20150117, |
| | | | | complete genome |
| AKU75313 | Sierra | Jan. 17, 2015 | Aug. 11, 2015 | Zaire ebolavirus isolate |
| | Leone | | | EBOV/DML24553/SLe/Kono/20150117, |
| | | | | complete genome |
| AKU75326 | Sierra | Jan. 18, 2015 | Aug. 11, 2015 | Zaire ebolavirus isolate |
| | Leone | | | EBOV/DML24573/SLe/Kono/20150118, |
| | | | | complete genome |
| AKU75331 | Sierra | Jan. 19, 2015 | Aug. 11, 2015 | Zaire ebolavirus isolate |
| | Leone | | | EBOV/DML24581/SLe/Kono/20150119, |
| | | | | complete genome |
| AKU75340 | Sierra | Jan. 20, 2015 | Aug. 11, 2015 | Zaire ebolavirus isolate |
| | Leone | | | EBOV/DML24592/SLe/Kono/20150120, |
| | | | | complete genome |
| AKU75351 | Sierra | Jan. 20, 2015 | Aug. 11, 2015 | Zaire ebolavirus isolate |
| | Leone | | | EBOV/DML24601/SLe/Kono/20150120, |
| | | | | complete genome |
| AKU75358 | Sierra | Jan. 20, 2015 | Aug. 11, 2015 | Zaire ebolavirus isolate |
| | Leone | | | EBOV/DML24604/SLe/Kono/20150120, |
| | | | | complete genome |
| AKU75367 | Sierra | Jan. 20, 2015 | Aug. 11, 2015 | Zaire ebolavirus isolate |
| | Leone | | | EBOV/DML24605/SLe/Kono/20150120, |
| | | | | complete genome |
| AKU75376 | Sierra | Jan. 20, 2015 | Aug. 11, 2015 | Zaire ebolavirus isolate |
| | Leone | | | EBOV/DML24606/SLe/Kono/20150120, |
| | | | | complete genome |
| AKU75385 | Sierra | Jan. 20, 2015 | Aug. 11, 2015 | Zaire ebolavirus isolate |
| | Leone | | | EBOV/DML24608/SLe/Kono/20150120, |
| | | | | complete genome |
| AKU75394 | Sierra | Jan. 21, 2015 | Aug. 11, 2015 | Zaire ebolavirus isolate |
| | Leone | | | EBOV/DML24611/SLe/Kono/20150121, |
| | | | | complete genome |
| AKU75403 | Sierra | Jan. 21, 2015 | Aug. 11, 2015 | Zaire ebolavirus isolate |
| | Leone | | | EBOV/DML24620/SLe/Kono/20150121, partial |
| | | | | genome |
| AKU75412 | Sierra | Jan. 25, 2015 | Aug. 11, 2015 | Zaire ebolavirus isolate |
| | Leone | | | EBOV/DML24669/SLe/Kono/20150125, |
| | | | | complete genome |
| AKU75421 | Sierra | Jan. 25, 2015 | Aug. 11, 2015 | Zaire ebolavirus isolate |
| | Leone | | | EBOV/DML24677/SLe/Kono/20150125, |
| | | | | complete genome |
| AKU75430 | Sierra | Jan. 26, 2015 | Aug. 11, 2015 | Zaire ebolavirus isolate |
| | Leone | | | EBOV/DML24683/SLe/Kono/20150126, |
| | | | | complete genome |
| AKU75439 | Sierra | Jan. 28, 2015 | Aug. 11, 2015 | Zaire ebolavirus isolate |
| | Leone | | | EBOV/DML24706/SLe/Kono/20150128, |
| | | | | complete genome |
| AKU75448 | Sierra | Jan. 28, 2015 | Aug. 11, 2015 | Zaire ebolavirus isolate |
| | Leone | | | EBOV/DML24708/SLe/Kono/20150128, |
| | | | | complete genome |
| AKU75457 | Sierra | Jan. 29, 2015 | Aug. 11, 2015 | Zaire ebolavirus isolate |
| | Leone | | | EBOV/DML24720/SLe/Kono/20150129, |
| | | | | complete genome |
| AKU75466 | Sierra | Jan. 30, 2015 | Aug. 11, 2015 | Zaire ebolavirus isolate |
| | Leone | | | EBOV/DML24758/SLe/Kono/20150130, |
| | | | | complete genome |
| AKU75475 | Sierra | Feb. 3, 2015 | Aug. 11, 2015 | Zaire ebolavirus isolate |
| | Leone | | | EBOV/DML24818/SLe/Kono/20150203, |
| | | | | complete genome |
| AKU75484 | Sierra | Feb. 4, 2015 | Aug. 11, 2015 | Zaire ebolavirus isolate |
| | Leone | | | EBOV/DML24825/SLe/Tonkolili/20150204, |
| | | | | complete genome |
| AKU75493 | Sierra | Feb. 6, 2015 | Aug. 11, 2015 | Zaire ebolavirus isolate |
| | Leone | | | EBOV/DML24853/SLe/Kono/20150206, |
| | | | | complete genome |
| AKU75502 | Sierra | Feb. 6, 2015 | Aug. 11, 2015 | Zaire ebolavirus isolate |
| | Leone | | | EBOV/DML24854/SLe/Kono/20150206, |
| | | | | complete genome |
| AKU75511 | Sierra | Feb. 18, 2015 | Aug. 11, 2015 | Zaire ebolavirus isolate |
| | Leone | | | EBOV/DML25083/SLe/Kono/20150218, |
| | | | | complete genome |
| AKU75520 | Sierra | Feb. 19, 2015 | Aug. 11, 2015 | Zaire ebolavirus isolate |
| | Leone | | | EBOV/DML25103/SLe/Kono/20150219, |
| | | | | complete genome |
| AKU75529 | Sierra | Feb. 18, 2015 | Aug. 11, 2015 | Zaire ebolavirus isolate |
| | Leone | | | EBOV/DML25123/SLe/Kenema/20150218, |
| | | | | partial genome |
| AKU75538 | Sierra | Feb. 23, 2015 | Aug. 11, 2015 | Zaire ebolavirus isolate |
| | Leone | | | EBOV/DML25180/SLe/Kono/20150223, |
| | | | | complete genome |
| AKU75547 | Sierra | Mar. 6, 2015 | Aug. 11, 2015 | Zaire ebolavirus isolate |
| | Leone | | | EBOV/DML25344/SLe/Kono/20150306, |
| | | | | complete genome |
| AKU75556 | Sierra | Mar. 10, 2015 | Aug. 11, 2015 | Zaire ebolavirus isolate |
| | Leone | | | EBOV/DML25411/SLe/Kono/20150310, partial |
| | | | | genome |
| AGB56840 | Democratic | 1976 | Jan. 7, 2013 | Zaire ebolavirus isolate EBOV/H.sapiens- |
| | Republic of | | | tc/COD/1976/deRoover, complete genome |
| | the Congo | | | |
| AGB56750 | Democratic | 1977 | Jan. 7, 2013 | Zaire ebolavirus isolate EBOV/H.sapiens- |
| | Republic of | | | tc/COD/1977/Bonduni, complete genome |
| | the Congo | | | |
| AGB56795 | Democratic | 1995 | Jan. 7, 2013 | Zaire ebolavirus isolate EBOV/H.sapiens- |
| | Republic of | | | tc/COD/1995/13625 Kikwit, complete genome |
| | the Congo | | | |
| AGB56822 | Democratic | 1995 | Jan. 7, 2013 | Zaire ebolavirus isolate EBOV/H.sapiens- |
| | Republic of | | | tc/COD/1995/13709 Kikwit, complete genome |
| | the Congo | | | |
| AGB56696 | Democratic | 2007 | Jan. 7, 2013 | Zaire ebolavirus isolate EBOV/H.sapiens- |
| | Republic of | | | tc/COD/2007/0 Luebo, complete genome |
| | the Congo | | | |
| AGB56705 | Democratic | 2007 | Jan. 7, 2013 | Zaire ebolavirus isolate EBOV/H.sapiens- |
| | Republic of | | | tc/COD/2007/1 Luebo, complete genome |
| | the Congo | | | |
| AGB56714 | Democratic | 2007 | Jan. 7, 2013 | Zaire ebolavirus isolate EBOV/H.sapiens- |
| | Republic of | | | tc/COD/2007/23 Luebo, complete genome |
| | the Congo | | | |
| AGB56732 | Democratic | 2007 | Jan. 7, 2013 | Zaire ebolavirus isolate EBOV/H.sapiens- |
| | Republic of | | | tc/COD/2007/4 Luebo, complete genome |
| | the Congo | | | |
| AGB56723 | Democratic | 2007 | Jan. 7, 2013 | Zaire ebolavirus isolate EBOV/H.sapiens- |
| | Republic of | | | tc/COD/2007/43 Luebo, complete genome |
| | the Congo | | | |
| AGB56741 | Democratic | 2007 | Jan. 7, 2013 | Zaire ebolavirus isolate EBOV/H.sapiens- |
| | Republic of | | | tc/COD/2007/5 Luebo, complete genome |
| | the Congo | | | |
| AGB56687 | Democratic | 2007 | Jan. 7, 2013 | Zaire ebolavirus isolate EBOV/H.sapiens- |
| | Republic of | | | tc/COD/2007/9 Luebo, complete genome |
| | the Congo | | | |
| AGB56759 | Gabon | 1994 | Jan. 7, 2013 | Zaire ebolavirus isolate EBOV/H.sapiens- |
| | | | | tc/GAB/1994/Gabon, complete genome |
| AGB56768 | Gabon | 1996 | Jan. 7, 2013 | Zaire ebolavirus isolate EBOV/H.sapiens- |
| | | | | tc/GAB/1996/1Eko, complete genome |
| AGB56813 | Gabon | 1996 | Jan. 7, 2013 | Zaire ebolavirus isolate EBOV/H.sapiens- |
| | | | | tc/GAB/1996/1Ikot, complete genome |
| AGB56786 | Gabon | 1996 | Jan. 7, 2013 | Zaire ebolavirus isolate EBOV/H.sapiens- |
| | | | | tc/GAB/1996/1Mbie, complete genome |
| AGB56804 | Gabon | 1996 | Jan. 7, 2013 | Zaire ebolavirus isolate EBOV/H.sapiens- |
| | | | | tc/GAB/1996/1Oba, complete genome |
| AGB56777 | Gabon | 1996 | Jan. 7, 2013 | Zaire ebolavirus isolate EBOV/H.sapiens- |
| | | | | tc/GAB/1996/2Nza, complete genome |
| AGB56831 | Gabon | 2002 | Jan. 7, 2013 | Zaire ebolavirus isolate EBOV/H.sapiens- |
| | | | | tc/GAB/2002/Ilembe, complete genome |
| AKA59361 | United | Mar. 12, 2015 | Apr. 4, 2015 | Zaire ebolavirus isolate Ebola virus H.sapiens- |
| | Kingdom | | | wt/GBR/2015/Makona-UK3, complete genome |
| AKC01436 | Liberia | 2014 | Apr. 14, 2015 | Zaire ebolavirus isolate Ebola virus H.sapiens- |
| | | | | wt/LBR/2014/Makona-Liberia-DQE1, complete |
| | | | | genome |
| AKC01476 | Liberia | 2014 | Apr. 14, 2015 | Zaire ebolavirus isolate Ebola virus H.sapiens- |
| | | | | wt/LBR/2014/Makona-Liberia-DQE12, complete |
| | | | | genome |
| AKC01484 | Liberia | 2014 | Apr. 14, 2015 | Zaire ebolavirus isolate Ebola virus H.sapiens- |
| | | | | wt/LBR/2014/Makona-Liberia-DQE13, complete |
| | | | | genome |
| AKC01492 | Liberia | 2014 | Apr. 14, 2015 | Zaire ebolavirus isolate Ebola virus H.sapiens- |
| | | | | wt/LBR/2014/Makona-Liberia-DQE14, complete |
| | | | | genome |
| AKC01444 | Liberia | 2015 | Apr. 14, 2015 | Zaire ebolavirus isolate Ebola virus H.sapiens- |
| | | | | wt/LBR/2015/Makona-Liberia-DQE3, complete |
| | | | | genome |
| AKC01452 | Liberia | 2015 | Apr. 14, 2015 | Zaire ebolavirus isolate Ebola virus H.sapiens- |
| | | | | wt/LBR/2015/Makona-Liberia-DQE4, complete |
| | | | | genome |
| AKC01460 | Liberia | 2015 | Apr. 14, 2015 | Zaire ebolavirus isolate Ebola virus H.sapiens- |
| | | | | wt/LBR/2015/Makona-Liberia-DQE5, complete |
| | | | | genome |
| AKC01468 | Liberia | 2015 | Apr. 14, 2015 | Zaire ebolavirus isolate Ebola virus H.sapiens- |
| | | | | wt/LBR/2015/Makona-Liberia-DQE6, complete |
| | | | | genome |
| AKT08841 | Sierra | Feb. 19, 2015 | Aug. 4, 2015 | Zaire ebolavirus isolate Ebola virus H.sapiens- |
| | Leone | | | wt/SLE/2015/Makona-Goderich1, complete |
| | | | | genome |
| AIO11752 | Democratic | 2014 | Oct. 17, 2014 | Zaire ebolavirus isolate Ebola virus/H.sap- |
| | Republic of | | | wt/COD/2014/Boende-Lokolia, partial genome |
| | the Congo | | | |
| AIR94007 | Democratic | 1976 | Oct. 3, 2014 | Zaire ebolavirus isolate Ebola virus/H.sapiens- |
| | Republic of | | | tc/COD/1976/Yambuku-Ecran, complete genome |
| | the Congo | | | |
| AIY29183 | United | Aug. 25, 2014 | Nov. 26, 2014 | Zaire ebolavirus isolate Ebola virus/H.sapiens- |
| | Kingdom | | | tc/GBR/2014/Makona-UK1.1, complete genome |
| AJF38896 | Italy | Nov. 25, 2014 | Feb. 1, 2015 | Zaire ebolavirus isolate Ebola virus/H.sapiens- |
| | | | | tc/SLE/2014/Makona-Italy-INMI1, complete |
| | | | | genome |
| AJG44193 | Switzerland | Nov. 21, 2014 | Feb. 9, 2015 | Zaire ebolavirus isolate Ebola virus/H.sapiens- |
| | | | | wt/CHE/2014/Makona-GE1, complete genome |
| AKI84248 | Democratic | Aug. 16, 2014 | Jul. 19, 2015 | Zaire ebolavirus isolate Ebola virus/H.sapiens- |
| | Republic of | | | wt/COD/2014/Lomela-Lokolia-B11, partial |
| | the Congo | | | genome |
| AJA04385 | Democratic | Aug. 20, 2014 | Dec. 22, 2014 | Zaire ebolavirus isolate Ebola virus/H.sapiens- |
| | Republic of | | | wt/COD/2014/Lomela-Lokolia16, complete |
| | the Congo | | | genome |
| AJA04394 | Democratic | Aug. 20, 2014 | Dec. 22, 2014 | Zaire ebolavirus isolate Ebola virus/H.sapiens- |
| | Republic of | | | wt/COD/2014/Lomela-Lokolia17, partial genome |
| | the Congo | | | |
| AJA04403 | Democratic | Aug. 20, 2014 | Dec. 22, 2014 | Zaire ebolavirus isolate Ebola virus/H.sapiens- |
| | Republic of | | | wt/COD/2014/Lomela-Lokolia19, complete |
| | the Congo | | | genome |
| AIW65951 | United | Aug. 25, 2014 | Nov. 15, 2014 | Zaire ebolavirus isolate Ebola virus/H.sapiens- |
| | Kingdom | | | wt/GBR/2014/Makona-UK1, complete genome |
| AJE60745 | United | Dec. 29, 2014 | Jan. 21, 2015 | Zaire ebolavirus isolate Ebola virus/H.sapiens- |
| | Kingdom | | | wt/GBR/2014/Makona-UK2, partial genome |
| AKL91083 | Guinea | Mar. 19, 2014 | Jun. 17, 2015 | Zaire ebolavirus isolate Ebola virus/H.sapiens- |
| | | | | wt/GIN/2014/Makona-C05, partial genome |
| AKL91092 | Guinea | Mar. 19, 2014 | Jun. 17, 2015 | Zaire ebolavirus isolate Ebola virus/H.sapiens- |
| | | | | wt/GIN/2014/Makona-C05, partial genome |
| AKL91101 | Guinea | Mar. 20, 2014 | Jun. 17, 2015 | Zaire ebolavirus isolate Ebola virus/H.sapiens- |
| | | | | wt/GIN/2014/Makona-C07, partial genome |
| AKL91110 | Guinea | Mar. 20, 2014 | Jun. 17, 2015 | Zaire ebolavirus isolate Ebola virus/H.sapiens- |
| | | | | wt/GIN/2014/Makona-C07, partial genome |
| AKL91119 | Guinea | Mar. 17, 2014 | Jun. 17, 2015 | Zaire ebolavirus isolate Ebola virus/H.sapiens- |
| | | | | wt/GIN/2014/Makona-C15, partial genome |
| AKL91128 | Guinea | Mar. 17, 2014 | Jun. 17, 2015 | Zaire ebolavirus isolate Ebola virus/H.sapiens- |
| | | | | wt/GIN/2014/Makona-C15, partial genome |
| AKG65728 | Guinea | Sep. 18, 2014 | Jun. 26, 2015 | Zaire ebolavirus isolate Ebola virus/H.sapiens- |
| | | | | wt/GIN/2014/Makona-Conakry-1027, partial |
| | | | | genome |
| AKG65737 | Guinea | Sep. 19, 2014 | Jun. 26, 2015 | Zaire ebolavirus isolate Ebola virus/H.sapiens- |
| | | | | wt/GIN/2014/Makona-Conakry-1039, partial |
| | | | | genome |
| AKG65278 | Guinea | Sep. 19, 2014 | Jun. 26, 2015 | Zaire ebolavirus isolate Ebola virus/H.sapiens- |
| | | | | wt/GIN/2014/Makona-Conakry-1043, partial |
| | | | | genome |
| AKG65296 | Guinea | Sep. 21, 2014 | Jun. 26, 2015 | Zaire ebolavirus isolate Ebola virus/H.sapiens- |
| | | | | wt/GIN/2014/Makona-Conakry-1059, partial |
| | | | | genome |
| AKG65323 | Guinea | Sep. 22, 2014 | Jun. 26, 2015 | Zaire ebolavirus isolate Ebola virus/H.sapiens- |
| | | | | wt/GIN/2014/Makona-Conakry-1081, partial |
| | | | | genome |
| AKG65746 | Guinea | Sep. 24, 2014 | Jun. 26, 2015 | Zaire ebolavirus isolate Ebola virus/H.sapiens- |
| | | | | wt/GIN/2014/Makona-Conakry-1105, partial |
| | | | | genome |
| AKG65350 | Guinea | Sep. 25, 2014 | Jun. 26, 2015 | Zaire ebolavirus isolate Ebola virus/H.sapiens- |
| | | | | wt/GIN/2014/Makona-Conakry-1120, partial |
| | | | | genome |
| AKG65359 | Guinea | Sep. 25, 2014 | Jun. 26, 2015 | Zaire ebolavirus isolate Ebola virus/H.sapiens- |
| | | | | wt/GIN/2014/Makona-Conakry-1121, partial |
| | | | | genome |
| AKG65755 | Guinea | Sep. 25, 2014 | Jun. 26, 2015 | Zaire ebolavirus isolate Ebola virus/H.sapiens- |
| | | | | wt/GIN/2014/Makona-Conakry-1128, partial |
| | | | | genome |
| AKG65764 | Guinea | Sep. 25, 2014 | Jun. 26, 2015 | Zaire ebolavirus isolate Ebola virus/H.sapiens- |
| | | | | wt/GIN/2014/Makona-Conakry-1129, partial |
| | | | | genome |
| AKG65773 | Guinea | Sep. 27, 2014 | Jun. 26, 2015 | Zaire ebolavirus isolate Ebola virus/H.sapiens- |
| | | | | wt/GIN/2014/Makona-Conakry-1149, partial |
| | | | | genome |
| AKG65368 | Guinea | Sep. 28, 2014 | Jun. 26, 2015 | Zaire ebolavirus isolate Ebola virus/H.sapiens- |
| | | | | wt/GIN/2014/Makona-Conakry-1193, partial |
| | | | | genome |
| AKG65377 | Guinea | Oct. 2, 2014 | Jun. 26, 2015 | Zaire ebolavirus isolate Ebola virus/H.sapiens- |
| | | | | wt/GIN/2014/Makona-Conakry-1205, partial |
| | | | | genome |
| AKG65386 | Guinea | Oct. 2, 2014 | Jun. 26, 2015 | Zaire ebolavirus isolate Ebola virus/H.sapiens- |
| | | | | wt/GIN/2014/Makona-Conakry-1210, partial |
| | | | | genome |
| AKG65782 | Guinea | Oct. 2, 2014 | Jun. 26, 2015 | Zaire ebolavirus isolate Ebola virus/H.sapiens- |
| | | | | wt/GIN/2014/Makona-Conakry-1213, partial |
| | | | | genome |
| AKG65791 | Guinea | Oct. 2, 2014 | Jun. 26, 2015 | Zaire ebolavirus isolate Ebola virus/H.sapiens- |
| | | | | wt/GIN/2014/Makona-Conakry-1215, partial |
| | | | | genome |
| AKG65404 | Guinea | Oct. 4, 2014 | Jun. 26, 2015 | Zaire ebolavirus isolate Ebola virus/H.sapiens- |
| | | | | wt/GIN/2014/Makona-Conakry-1249, partial |
| | | | | genome |
| AKG65800 | Guinea | Oct. 4, 2014 | Jun. 26, 2015 | Zaire ebolavirus isolate Ebola virus/H.sapiens- |
| | | | | wt/GIN/2014/Makona-Conakry-1250, partial |
| | | | | genome |
| AKG65440 | Guinea | Oct. 6, 2014 | Jun. 26, 2015 | Zaire ebolavirus isolate Ebola virus/H.sapiens- |
| | | | | wt/GIN/2014/Makona-Conakry-1298, partial |
| | | | | genome |
| AKG65494 | Guinea | Oct. 8, 2014 | Jun. 26, 2015 | Zaire ebolavirus isolate Ebola virus/H.sapiens- |
| | | | | wt/GIN/2014/Makona-Conakry-1340, partial |
| | | | | genome |
| AKG65503 | Guinea | Oct. 8, 2014 | Jun. 26, 2015 | Zaire ebolavirus isolate Ebola virus/H.sapiens- |
| | | | | wt/GIN/2014/Makona-Conakry-1342, partial |
| | | | | genome |
| AKG65530 | Guinea | Oct. 10, 2014 | Jun. 26, 2015 | Zaire ebolavirus isolate Ebola virus/H.sapiens- |
| | | | | wt/GIN/2014/Makona-Conakry-1371, partial |
| | | | | genome |
| AKG65566 | Guinea | Oct. 14, 2014 | Jun. 26, 2015 | Zaire ebolavirus isolate Ebola virus/H.sapiens- |
| | | | | wt/GIN/2014/Makona-Conakry-1445, partial |
| | | | | genome |
| AKG65575 | Guinea | Oct. 14, 2014 | Jun. 26, 2015 | Zaire ebolavirus isolate Ebola virus/H.sapiens- |
| | | | | wt/GIN/2014/Makona-Conakry-1454, partial |
| | | | | genome |
| AKG65584 | Guinea | Oct. 15, 2014 | Jun. 26, 2015 | Zaire ebolavirus isolate Ebola virus/H.sapiens- |
| | | | | wt/GIN/2014/Makona-Conakry-1480, partial |
| | | | | genome |
| AKG65710 | Guinea | Oct. 15, 2014 | Jun. 26, 2015 | Zaire ebolavirus isolate Ebola virus/H.sapiens- |
| | | | | wt/GIN/2014/Makona-Conakry-1481, partial |
| | | | | genome |
| AKG65719 | Guinea | Oct. 16, 2014 | Jun. 26, 2015 | Zaire ebolavirus isolate Ebola virus/H.sapiens- |
| | | | | wt/GIN/2014/Makona-Conakry-1491, partial |
| | | | | genome |
| AKG65593 | Guinea | Oct. 18, 2014 | Jun. 26, 2015 | Zaire ebolavirus isolate Ebola virus/H.sapiens- |
| | | | | wt/GIN/2014/Makona-Conakry-1551, partial |
| | | | | genome |
| AKG65602 | Guinea | Oct. 19, 2014 | Jun. 26, 2015 | Zaire ebolavirus isolate Ebola virus/H.sapiens- |
| | | | | wt/GIN/2014/Makona-Conakry-1561, partial |
| | | | | genome |
| AKG65665 | Guinea | Oct. 24, 2014 | Jun. 26, 2015 | Zaire ebolavirus isolate Ebola virus/H.sapiens- |
| | | | | wt/GIN/2014/Makona-Conakry-1651, partial |
| | | | | genome |
| ALF04602 | Guinea | Oct. 13, 2014 | Sep. 26, 2015 | Zaire ebolavirus isolate Ebola virus/H.sapiens- |
| | | | | wt/GIN/2014/Makona-Conakry-211, partial |
| | | | | genome |
| ALF04596 | Guinea | Oct. 14, 2014 | Sep. 26, 2015 | Zaire ebolavirus isolate Ebola virus/H.sapiens- |
| | | | | wt/GIN/2014/Makona-Conakry-223, partial |
| | | | | genome |
| AKG65097 | Guinea | Jul. 24, 2014 | Jun. 26, 2015 | Zaire ebolavirus isolate Ebola virus/H.sapiens- |
| | | | | wt/GIN/2014/Makona-Conakry-505, partial |
| | | | | genome |
| AKG65809 | Guinea | Jul. 24, 2014 | Jun. 26, 2015 | Zaire ebolavirus isolate Ebola virus/H.sapiens- |
| | | | | wt/GIN/2014/Makona-Conakry-507, partial |
| | | | | genome |
| AKG65107 | Guinea | Jul. 24, 2014 | Jun. 26, 2015 | Zaire ebolavirus isolate Ebola virus/H.sapiens- |
| | | | | wt/GIN/2014/Makona-Conakry-509, partial |
| | | | | genome |
| AKG65134 | Guinea | Aug. 2, 2014 | Jun. 26, 2015 | Zaire ebolavirus isolate Ebola virus/H.sapiens- |
| | | | | wt/GIN/2014/Makona-Conakry-573, partial |
| | | | | genome |
| AKG65161 | Guinea | Aug. 15, 2014 | Jun. 26, 2015 | Zaire ebolavirus isolate Ebola virus/H.sapiens- |
| | | | | wt/GIN/2014/Makona-Conakry-653, partial |
| | | | | genome |
| AKG65170 | Guinea | Aug. 16, 2014 | Jun. 26, 2015 | Zaire ebolavirus isolate Ebola virus/H.sapiens- |
| | | | | wt/GIN/2014/Makona-Conakry-657, partial |
| | | | | genome |
| AKG65179 | Guinea | Aug. 19, 2014 | Jun. 26, 2015 | Zaire ebolavirus isolate Ebola virus/H.sapiens- |
| | | | | wt/GIN/2014/Makona-Conakry-678, partial |
| | | | | genome |
| AKG65188 | Guinea | Aug. 20, 2014 | Jun. 26, 2015 | Zaire ebolavirus isolate Ebola virus/H.sapiens- |
| | | | | wt/GIN/2014/Makona-Conakry-684, partial |
| | | | | genome |
| AKG65197 | Guinea | Aug. 21, 2014 | Jun. 26, 2015 | Zaire ebolavirus isolate Ebola virus/H.sapiens- |
| | | | | wt/GIN/2014/Makona-Conakry-691, partial |
| | | | | genome |
| AKG65206 | Guinea | Aug. 22, 2014 | Jun. 26, 2015 | Zaire ebolavirus isolate Ebola virus/H.sapiens- |
| | | | | wt/GIN/2014/Makona-Conakry-701, partial |
| | | | | genome |
| AKG65215 | Guinea | Aug. 26, 2014 | Jun. 26, 2015 | Zaire ebolavirus isolate Ebola virus/H.sapiens- |
| | | | | wt/GIN/2014/Makona-Conakry-740, partial |
| | | | | genome |
| AKG65818 | Guinea | Aug. 26, 2014 | Jun. 26, 2015 | Zaire ebolavirus isolate Ebola virus/H.sapiens- |
| | | | | wt/GIN/2014/Makona-Conakry-742, partial |
| | | | | genome |
| AKG65827 | Guinea | Aug. 27, 2014 | Jun. 26, 2015 | Zaire ebolavirus isolate Ebola virus/H.sapiens- |
| | | | | wt/GIN/2014/Makona-Conakry-768, partial |
| | | | | genome |
| AKG65224 | Guinea | Aug. 28, 2014 | Jun. 26, 2015 | Zaire ebolavirus isolate Ebola virus/H.sapiens- |
| | | | | wt/GIN/2014/Makona-Conakry-786, partial |
| | | | | genome |
| AKG65233 | Guinea | Aug. 28, 2014 | Jun. 26, 2015 | Zaire ebolavirus isolate Ebola virus/H.sapiens- |
| | | | | wt/GIN/2014/Makona-Conakry-787, partial |
| | | | | genome |
| AKG65260 | Guinea | Sep. 14, 2014 | Jun. 26, 2015 | Zaire ebolavirus isolate Ebola virus/H.sapiens- |
| | | | | wt/GIN/2014/Makona-Conakry-976, partial |
| | | | | genome |
| AKG65305 | Guinea | Sep. 21, 2014 | Jun. 26, 2015 | Zaire ebolavirus isolate Ebola virus/H.sapiens- |
| | | | | wt/GIN/2014/Makona-Coyah-1063, partial |
| | | | | genome |
| AKG65413 | Guinea | Oct. 4, 2014 | Jun. 26, 2015 | Zaire ebolavirus isolate Ebola virus/H.sapiens- |
| | | | | wt/GIN/2014/Makona-Coyah-1274, partial |
| | | | | genome |
| AKG65422 | Guinea | Oct. 4, 2014 | Jun. 26, 2015 | Zaire ebolavirus isolate Ebola virus/H.sapiens- |
| | | | | wt/GIN/2014/Makona-Coyah-1277, partial |
| | | | | genome |
| AKG65431 | Guinea | Oct. 4, 2014 | Jun. 26, 2015 | Zaire ebolavirus isolate Ebola virus/H.sapiens- |
| | | | | wt/GIN/2014/Makona-Coyah-1278, partial |
| | | | | genome |
| AKG65836 | Guinea | Oct. 4, 2014 | Jun. 26, 2015 | Zaire ebolavirus isolate Ebola virus/H.sapiens- |
| | | | | wt/GIN/2014/Makona-Coyah-1279, partial |
| | | | | genome |
| AKG65449 | Guinea | Oct. 7, 2014 | Jun. 26, 2015 | Zaire ebolavirus isolate Ebola virus/H.sapiens- |
| | | | | wt/GIN/2014/Makona-Coyah-1316, partial |
| | | | | genome |
| AKG65845 | Guinea | Oct. 7, 2014 | Jun. 26, 2015 | Zaire ebolavirus isolate Ebola virus/H.sapiens- |
| | | | | wt/GIN/2014/Makona-Coyah-1320, partial |
| | | | | genome |
| AKG65458 | Guinea | Oct. 7, 2014 | Jun. 26, 2015 | Zaire ebolavirus isolate Ebola virus/H.sapiens- |
| | | | | wt/GIN/2014/Makona-Coyah-1321, partial |
| | | | | genome |
| AKG65854 | Guinea | Oct. 7, 2014 | Jun. 26, 2015 | Zaire ebolavirus isolate Ebola virus/H.sapiens- |
| | | | | wt/GIN/2014/Makona-Coyah-1327, partial |
| | | | | genome |
| AKG65476 | Guinea | Oct. 7, 2014 | Jun. 26, 2015 | Zaire ebolavirus isolate Ebola virus/H.sapiens- |
| | | | | wt/GIN/2014/Makona-Coyah-1333, partial |
| | | | | genome |
| AKG65485 | Guinea | Oct. 8, 2014 | Jun. 26, 2015 | Zaire ebolavirus isolate Ebola virus/H.sapiens- |
| | | | | wt/GIN/2014/Makona-Coyah-1339, partial |
| | | | | genome |
| AKG65512 | Guinea | Oct. 9, 2014 | Jun. 26, 2015 | Zaire ebolavirus isolate Ebola virus/H.sapiens- |
| | | | | wt/GIN/2014/Makona-Coyah-1355, partial |
| | | | | genome |
| AKG65539 | Guinea | Oct. 10, 2014 | Jun. 26, 2015 | Zaire ebolavirus isolate Ebola virus/H.sapiens- |
| | | | | wt/GIN/2014/Makona-Coyah-1374, partial |
| | | | | genome |
| AKG65548 | Guinea | Oct. 11, 2014 | Jun. 26, 2015 | Zaire ebolavirus isolate Ebola virus/H.sapiens- |
| | | | | wt/GIN/2014/Makona-Coyah-1394, partial |
| | | | | genome |
| AKG65557 | Guinea | Oct. 13, 2014 | Jun. 26, 2015 | Zaire ebolavirus isolate Ebola virus/H.sapiens- |
| | | | | wt/GIN/2014/Makona-Coyah-1436, partial |
| | | | | genome |
| AKG65674 | Guinea | Oct. 24, 2014 | Jun. 26, 2015 | Zaire ebolavirus isolate Ebola virus/H.sapiens- |
| | | | | wt/GIN/2014/Makona-Coyah-1652, partial |
| | | | | genome |
| AKG65683 | Guinea | Oct. 24, 2014 | Jun. 26, 2015 | Zaire ebolavirus isolate Ebola virus/H.sapiens- |
| | | | | wt/GIN/2014/Makona-Coyah-1686, partial |
| | | | | genome |
| AKG65692 | Guinea | Oct. 25, 2014 | Jun. 26, 2015 | Zaire ebolavirus isolate Ebola virus/H.sapiens- |
| | | | | wt/GIN/2014/Makona-Coyah-1689, partial |
| | | | | genome |
| AKG65701 | Guinea | Oct. 25, 2014 | Jun. 26, 2015 | Zaire ebolavirus isolate Ebola virus/H.sapiens- |
| | | | | wt/GIN/2014/Makona-Coyah-1690, partial |
| | | | | genome |
| AKG65251 | Guinea | Sep. 11, 2014 | Jun. 26, 2015 | Zaire ebolavirus isolate Ebola virus/H.sapiens- |
| | | | | wt/GIN/2014/Makona-Coyah-955, partial genome |
| AKG65332 | Guinea | Sep. 24, 2014 | Jun. 26, 2015 | Zaire ebolavirus isolate Ebola virus/H.sapiens- |
| | | | | wt/GIN/2014/Makona-Dalaba-1104, partial |
| | | | | genome |
| AKG65341 | Guinea | Sep. 24, 2014 | Jun. 26, 2015 | Zaire ebolavirus isolate Ebola virus/H.sapiens- |
| | | | | wt/GIN/2014/Makona-Dalaba-1116, partial |
| | | | | genome |
| AKG65395 | Guinea | Oct. 2, 2014 | Jun. 26, 2015 | Zaire ebolavirus isolate Ebola virus/H.sapiens- |
| | | | | wt/GIN/2014/Makona-Dalaba-1211, partial |
| | | | | genome |
| AKG65242 | Guinea | Aug. 29, 2014 | Jun. 26, 2015 | Zaire ebolavirus isolate Ebola virus/H.sapiens- |
| | | | | wt/GIN/2014/Makona-Dubreka-789, partial |
| | | | | genome |
| AKI82636 | Guinea | Sep. 1, 2014 | Jun. 12, 2015 | Zaire ebolavirus isolate Ebola virus/H.sapiens- |
| | | | | wt/GIN/2014/Makona-EM_000015, partial |
| | | | | genome |
| AKI82645 | Guinea | Sep. 1, 2014 | Jun. 12, 2015 | Zaire ebolavirus isolate Ebola virus/H.sapiens- |
| | | | | wt/GIN/2014/Makona-EM_000027, partial |
| | | | | genome |
| AKI82654 | Guinea | Sep. 1, 2014 | Jun. 12, 2015 | Zaire ebolavirus isolate Ebola virus/H.sapiens- |
| | | | | wt/GIN/2014/Makona-EM_000028, partial |
| | | | | genome |
| AKI82663 | Guinea | Sep. 4, 2014 | Jun. 12, 2015 | Zaire ebolavirus isolate Ebola virus/H.sapiens- |
| | | | | wt/GIN/2014/Makona-EM_000127, partial |
| | | | | genome |
| AKI82672 | Guinea | Sep. 4, 2014 | Jun. 12, 2015 | Zaire ebolavirus isolate Ebola virus/H.sapiens- |
| | | | | wt/GIN/2014/Makona-EM_000128, partial |
| | | | | genome |
| AKI82681 | Guinea | Sep. 7, 2014 | Jun. 12, 2015 | Zaire ebolavirus isolate Ebola virus/H.sapiens- |
| | | | | wt/GIN/2014/Makona-EM_000218, partial |
| | | | | genome |
| AKI82690 | Guinea | Sep. 7, 2014 | Jun. 12, 2015 | Zaire ebolavirus isolate Ebola virus/H.sapiens- |
| | | | | wt/GIN/2014/Makona-EM_000219, partial |
| | | | | genome |
| AKI82699 | Guinea | Sep. 9, 2014 | Jun. 12, 2015 | Zaire ebolavirus isolate Ebola virus/H.sapiens- |
| | | | | wt/GIN/2014/Makona-EM_000321, partial |
| | | | | genome |
| AKI82708 | Guinea | Sep. 12, 2014 | Jun. 12, 2015 | Zaire ebolavirus isolate Ebola virus/H.sapiens- |
| | | | | wt/GIN/2014/Makona-EM_000457, partial |
| | | | | genome |
| AKI82717 | Guinea | Sep. 13, 2014 | Jun. 12, 2015 | Zaire ebolavirus isolate Ebola virus/H.sapiens- |
| | | | | wt/GIN/2014/Makona-EM_000500, partial |
| | | | | genome |
| AKI82726 | Guinea | Sep. 13, 2014 | Jun. 12, 2015 | Zaire ebolavirus isolate Ebola virus/H.sapiens- |
| | | | | wt/GIN/2014/Makona-EM_000501, partial |
| | | | | genome |
| AKI82735 | Guinea | Sep. 13, 2014 | Jun. 12, 2015 | Zaire ebolavirus isolate Ebola virus/H.sapiens- |
| | | | | wt/GIN/2014/Makona-EM_000502, partial |
| | | | | genome |
| AKI82744 | Guinea | Sep. 21, 2014 | Jun. 12, 2015 | Zaire ebolavirus isolate Ebola virus/H.sapiens- |
| | | | | wt/GIN/2014/Makona-EM_000706, partial |
| | | | | genome |
| AKI82753 | Guinea | Sep. 22, 2014 | Jun. 12, 2015 | Zaire ebolavirus isolate Ebola virus/H.sapiens- |
| | | | | wt/GIN/2014/Makona-EM_000707, partial |
| | | | | genome |
| AKI82762 | Guinea | Sep. 29, 2014 | Jun. 12, 2015 | Zaire ebolavirus isolate Ebola virus/H.sapiens- |
| | | | | wt/GIN/2014/Makona-EM_000921, partial |
| | | | | genome |
| AKI82771 | Guinea | Sep. 29, 2014 | Jun. 12, 2015 | Zaire ebolavirus isolate Ebola virus/H.sapiens- |
| | | | | wt/GIN/2014/Makona-EM_000925, partial |
| | | | | genome |
| AKI82780 | Guinea | Sep. 29, 2014 | Jun. 12, 2015 | Zaire ebolavirus isolate Ebola virus/H.sapiens- |
| | | | | wt/GIN/2014/Makona-EM_000934, partial |
| | | | | genome |
| AKI82789 | Guinea | Sep. 30, 2014 | Jun. 12, 2015 | Zaire ebolavirus isolate Ebola virus/H.sapiens- |
| | | | | wt/GIN/2014/Makona-EM_000958, partial |
| | | | | genome |
| AKI82798 | Guinea | Oct. 1, 2014 | Jun. 12, 2015 | Zaire ebolavirus isolate Ebola virus/H.sapiens- |
| | | | | wt/GIN/2014/Makona-EM_000968, partial |
| | | | | genome |
| AKI82807 | Guinea | Oct. 1, 2014 | Jun. 12, 2015 | Zaire ebolavirus isolate Ebola virus/H.sapiens- |
| | | | | wt/GIN/2014/Makona-EM_000982, partial |
| | | | | genome |
| AKI82816 | Guinea | Oct. 1, 2014 | Jun. 12, 2015 | Zaire ebolavirus isolate Ebola virus/H.sapiens- |
| | | | | wt/GIN/2014/Makona-EM_000983, partial |
| | | | | genome |
| AKI82825 | Guinea | Oct. 5, 2014 | Jun. 12, 2015 | Zaire ebolavirus isolate Ebola virus/H.sapiens- |
| | | | | wt/GIN/2014/Makona-EM_001101, partial |
| | | | | genome |
| AKI82834 | Guinea | Oct. 5, 2014 | Jun. 12, 2015 | Zaire ebolavirus isolate Ebola virus/H.sapiens- |
| | | | | wt/GIN/2014/Makona-EM_001102, partial |
| | | | | genome |
| AKI82843 | Guinea | Dec. 20, 2014 | Jun. 12, 2015 | Zaire ebolavirus isolate Ebola virus/H.sapiens- |
| | | | | wt/GIN/2014/Makona-EM_004059, partial |
| | | | | genome |
| AKI82852 | Guinea | Dec. 19, 2014 | Jun. 12, 2015 | Zaire ebolavirus isolate Ebola virus/H.sapiens- |
| | | | | wt/GIN/2014/Makona-EM_004085, partial |
| | | | | genome |
| AKI82861 | Guinea | Dec. 22, 2014 | Jun. 12, 2015 | Zaire ebolavirus isolate Ebola virus/H.sapiens- |
| | | | | wt/GIN/2014/Makona-EM_004192, partial |
| | | | | genome |
| AKI82870 | Guinea | Dec. 24, 2014 | Jun. 12, 2015 | Zaire ebolavirus isolate Ebola virus/H.sapiens- |
| | | | | wt/GIN/2014/Makona-EM_004201, partial |
| | | | | genome |
| AKI82879 | Guinea | Dec. 26, 2014 | Jun. 12, 2015 | Zaire ebolavirus isolate Ebola virus/H.sapiens- |
| | | | | wt/GIN/2014/Makona-EM_004259, partial |
| | | | | genome |
| AKI82888 | Guinea | Dec. 27, 2014 | Jun. 12, 2015 | Zaire ebolavirus isolate Ebola virus/H.sapiens- |
| | | | | wt/GIN/2014/Makona-EM_004290, partial |
| | | | | genome |
| AKI82996 | Guinea | Jul. 18, 2014 | Jun. 12, 2015 | Zaire ebolavirus isolate Ebola virus/H.sapiens- |
| | | | | wt/GIN/2014/Makona-EM_074335, partial |
| | | | | genome |
| AKI83050 | Guinea | Jul. 22, 2014 | Jun. 12, 2015 | Zaire ebolavirus isolate Ebola virus/H.sapiens- |
| | | | | wt/GIN/2014/Makona-EM_074354, partial |
| | | | | genome |
| AKI83077 | Guinea | Aug. 1, 2014 | Jun. 12, 2015 | Zaire ebolavirus isolate Ebola virus/H.sapiens- |
| | | | | wt/GIN/2014/Makona-EM_074436, partial |
| | | | | genome |
| AKI83086 | Guinea | Aug. 1, 2014 | Jun. 12, 2015 | Zaire ebolavirus isolate Ebola virus/H.sapiens- |
| | | | | wt/GIN/2014/Makona-EM_074437, partial |
| | | | | genome |
| AKI83095 | Guinea | Aug. 1, 2014 | Jun. 12, 2015 | Zaire ebolavirus isolate Ebola virus/H.sapiens- |
| | | | | wt/GIN/2014/Makona-EM_074438, partial |
| | | | | genome |
| AKI83104 | Guinea | Aug. 1, 2014 | Jun. 12, 2015 | Zaire ebolavirus isolate Ebola virus/H.sapiens- |
| | | | | wt/GIN/2014/Makona-EM_074439, partial |
| | | | | genome |
| AKI83113 | Guinea | Aug. 4, 2014 | Jun. 12, 2015 | Zaire ebolavirus isolate Ebola virus/H.sapiens- |
| | | | | wt/GIN/2014/Makona-EM_074461, partial |
| | | | | genome |
| AKI83122 | Guinea | Aug. 4, 2014 | Jun. 12, 2015 | Zaire ebolavirus isolate Ebola virus/H.sapiens- |
| | | | | wt/GIN/2014/Makona-EM_074462, partial |
| | | | | genome |
| AKI83131 | Guinea | Aug. 8, 2014 | Jun. 12, 2015 | Zaire ebolavirus isolate Ebola virus/H.sapiens- |
| | | | | wt/GIN/2014/Makona-EM_074531, partial |
| | | | | genome |
| AKI83149 | Guinea | Aug. 14, 2014 | Jun. 12, 2015 | Zaire ebolavirus isolate Ebola virus/H.sapiens- |
| | | | | wt/GIN/2014/Makona-EM_074684, partial |
| | | | | genome |
| AKI83167 | Guinea | Aug. 16, 2014 | Jun. 12, 2015 | Zaire ebolavirus isolate Ebola virus/H.sapiens- |
| | | | | wt/GIN/2014/Makona-EM_074785, partial |
| | | | | genome |
| AKI83203 | Guinea | Aug. 23, 2014 | Jun. 12, 2015 | Zaire ebolavirus isolate Ebola virus/H.sapiens- |
| | | | | wt/GIN/2014/Makona-EM_075076, partial |
| | | | | genome |
| AKI83212 | Guinea | Aug. 29, 2014 | Jun. 12, 2015 | Zaire ebolavirus isolate Ebola virus/H.sapiens- |
| | | | | wt/GIN/2014/Makona-EM_075368, partial |
| | | | | genome |
| AKI83221 | Guinea | Aug. 30, 2014 | Jun. 12, 2015 | Zaire ebolavirus isolate Ebola virus/H.sapiens- |
| | | | | wt/GIN/2014/Makona-EM_075373, partial |
| | | | | genome |
| AKI83230 | Guinea | Aug. 30, 2014 | Jun. 12, 2015 | Zaire ebolavirus isolate Ebola virus/H.sapiens- |
| | | | | wt/GIN/2014/Makona-EM_075435, partial |
| | | | | genome |
| AKI83239 | Guinea | Aug. 31, 2014 | Jun. 12, 2015 | Zaire ebolavirus isolate Ebola virus/H.sapiens- |
| | | | | wt/GIN/2014/Makona-EM_075447, partial |
| | | | | genome |
| AKI83248 | Guinea | Oct. 10, 2014 | Jun. 12, 2015 | Zaire ebolavirus isolate Ebola virus/H.sapiens- |
| | | | | wt/GIN/2014/Makona-EM_075928, partial |
| | | | | genome |
| AKI83257 | Guinea | Oct. 10, 2014 | Jun. 12, 2015 | Zaire ebolavirus isolate Ebola virus/H.sapiens- |
| | | | | wt/GIN/2014/Makona-EM_075929, partial |
| | | | | genome |
| AKI83266 | Guinea | Oct. 10, 2014 | Jun. 12, 2015 | Zaire ebolavirus isolate Ebola virus/H.sapiens- |
| | | | | wt/GIN/2014/Makona-EM_075930, partial |
| | | | | genome |
| AKI83275 | Guinea | Oct. 10, 2014 | Jun. 12, 2015 | Zaire ebolavirus isolate Ebola virus/H.sapiens- |
| | | | | wt/GIN/2014/Makona-EM_075931, partial |
| | | | | genome |
| AKI83284 | Guinea | Oct. 10, 2014 | Jun. 12, 2015 | Zaire ebolavirus isolate Ebola virus/H.sapiens- |
| | | | | wt/GIN/2014/Makona-EM_075932, partial |
| | | | | genome |
| AKI83293 | Guinea | Oct. 17, 2014 | Jun. 12, 2015 | Zaire ebolavirus isolate Ebola virus/H.sapiens- |
| | | | | wt/GIN/2014/Makona-EM_076138, partial |
| | | | | genome |
| AKI83302 | Guinea | Oct. 18, 2014 | Jun. 12, 2015 | Zaire ebolavirus isolate Ebola virus/H.sapiens- |
| | | | | wt/GIN/2014/Makona-EM_076191, partial |
| | | | | genome |
| AKI83311 | Guinea | Oct. 18, 2014 | Jun. 12, 2015 | Zaire ebolavirus isolate Ebola virus/H.sapiens- |
| | | | | wt/GIN/2014/Makona-EM_076192, partial |
| | | | | genome |
| AKI83320 | Guinea | Oct. 18, 2014 | Jun. 12, 2015 | Zaire ebolavirus isolate Ebola virus/H.sapiens- |
| | | | | wt/GIN/2014/Makona-EM_076193, partial |
| | | | | genome |
| AKI83329 | Guinea | Oct. 19, 2014 | Jun. 12, 2015 | Zaire ebolavirus isolate Ebola virus/H.sapiens- |
| | | | | wt/GIN/2014/Makona-EM_076217, partial |
| | | | | genome |
| AKI83338 | Guinea | Oct. 22, 2014 | Jun. 12, 2015 | Zaire ebolavirus isolate Ebola virus/H.sapiens- |
| | | | | wt/GIN/2014/Makona-EM_076322, partial |
| | | | | genome |
| AKI83347 | Guinea | Oct. 23, 2014 | Jun. 12, 2015 | Zaire ebolavirus isolate Ebola virus/H.sapiens- |
| | | | | wt/GIN/2014/Makona-EM_076334, partial |
| | | | | genome |
| AKI83356 | Guinea | Oct. 23, 2014 | Jun. 12, 2015 | Zaire ebolavirus isolate Ebola virus/H.sapiens- |
| | | | | wt/GIN/2014/Makona-EM_076335, partial |
| | | | | genome |
| AKI83365 | Guinea | Oct. 26, 2014 | Jun. 12, 2015 | Zaire ebolavirus isolate Ebola virus/H.sapiens- |
| | | | | wt/GIN/2014/Makona-EM_076383, partial |
| | | | | genome |
| AKI83374 | Guinea | Oct. 27, 2014 | Jun. 12, 2015 | Zaire ebolavirus isolate Ebola virus/H.sapiens- |
| | | | | wt/GIN/2014/Makona-EM_076403, partial |
| | | | | genome |
| AKI83383 | Guinea | Oct. 29, 2014 | Jun. 12, 2015 | Zaire ebolavirus isolate Ebola virus/H.sapiens- |
| | | | | wt/GIN/2014/Makona-EM_076472, partial |
| | | | | genome |
| AKI83392 | Guinea | Nov. 1, 2014 | Jun. 12, 2015 | Zaire ebolavirus isolate Ebola virus/H.sapiens- |
| | | | | wt/GIN/2014/Makona-EM_076533, partial |
| | | | | genome |
| AKI83401 | Guinea | Nov. 1, 2014 | Jun. 12, 2015 | Zaire ebolavirus isolate Ebola virus/H.sapiens- |
| | | | | wt/GIN/2014/Makona-EM_076534, partial |
| | | | | genome |
| AKI83410 | Guinea | Nov. 3, 2014 | Jun. 12, 2015 | Zaire ebolavirus isolate Ebola virus/H.sapiens- |
| | | | | wt/GIN/2014/Makona-EM_076610, partial |
| | | | | genome |
| AKI83419 | Guinea | Nov. 3, 2014 | Jun. 12, 2015 | Zaire ebolavirus isolate Ebola virus/H.sapiens- |
| | | | | wt/GIN/2014/Makona-EM_076615, partial |
| | | | | genome |
| AKI83428 | Guinea | Nov. 8, 2014 | Jun. 12, 2015 | Zaire ebolavirus isolate Ebola virus/H.sapiens- |
| | | | | wt/GIN/2014/Makona-EM_076769, partial |
| | | | | genome |
| AKI83437 | Guinea | Nov. 8, 2014 | Jun. 12, 2015 | Zaire ebolavirus isolate Ebola virus/H.sapiens- |
| | | | | wt/GIN/2014/Makona-EM_076770, partial |
| | | | | genome |
| AKI83446 | Guinea | Nov. 13, 2014 | Jun. 12, 2015 | Zaire ebolavirus isolate Ebola virus/H.sapiens- |
| | | | | wt/GIN/2014/Makona-EM_076948, partial |
| | | | | genome |
| AKI83455 | Guinea | Nov. 13, 2014 | Jun. 12, 2015 | Zaire ebolavirus isolate Ebola virus/H.sapiens- |
| | | | | wt/GIN/2014/Makona-EM_076949, partial |
| | | | | genome |
| AKI83464 | Guinea | Nov. 13, 2014 | Jun. 12, 2015 | Zaire ebolavirus isolate Ebola virus/H.sapiens- |
| | | | | wt/GIN/2014/Makona-EM_076951, partial |
| | | | | genome |
| AKI83473 | Guinea | Nov. 24, 2014 | Jun. 12, 2015 | Zaire ebolavirus isolate Ebola virus/H.sapiens- |
| | | | | wt/GIN/2014/Makona-EM_078415, partial |
| | | | | genome |
| AKI83482 | Guinea | Nov. 24, 2014 | Jun. 12, 2015 | Zaire ebolavirus isolate Ebola virus/H.sapiens- |
| | | | | wt/GIN/2014/Makona-EM_078416, partial |
| | | | | genome |
| AKI83491 | Guinea | Dec. 1, 2014 | Jun. 12, 2015 | Zaire ebolavirus isolate Ebola virus/H.sapiens- |
| | | | | wt/GIN/2014/Makona-EM_078555, partial |
| | | | | genome |
| AKI83500 | Guinea | Dec. 1, 2014 | Jun. 12, 2015 | Zaire ebolavirus isolate Ebola virus/H.sapiens- |
| | | | | wt/GIN/2014/Makona-EM_078556, partial |
| | | | | genome |
| AKI83509 | Guinea | Dec. 4, 2014 | Jun. 12, 2015 | Zaire ebolavirus isolate Ebola virus/H.sapiens- |
| | | | | wt/GIN/2014/Makona-EM_078608, partial |
| | | | | genome |
| AKI83518 | Guinea | Dec. 3, 2014 | Jun. 12, 2015 | Zaire ebolavirus isolate Ebola virus/H.sapiens- |
| | | | | wt/GIN/2014/Makona-EM_078638, partial |
| | | | | genome |
| AKI83527 | Guinea | Dec. 4, 2014 | Jun. 12, 2015 | Zaire ebolavirus isolate Ebola virus/H.sapiens- |
| | | | | wt/GIN/2014/Makona-EM_078639, partial |
| | | | | genome |
| AKI83536 | Guinea | Dec. 9, 2014 | Jun. 12, 2015 | Zaire ebolavirus isolate Ebola virus/H.sapiens- |
| | | | | wt/GIN/2014/Makona-EM_078654, partial |
| | | | | genome |
| AKI83545 | Guinea | Dec. 9, 2014 | Jun. 12, 2015 | Zaire ebolavirus isolate Ebola virus/H.sapiens- |
| | | | | wt/GIN/2014/Makona-EM_078656, partial |
| | | | | genome |
| AKI83554 | Guinea | Dec. 11, 2014 | Jun. 12, 2015 | Zaire ebolavirus isolate Ebola virus/H.sapiens- |
| | | | | wt/GIN/2014/Makona-EM_078670, partial |
| | | | | genome |
| AKI83563 | Guinea | Dec. 11, 2014 | Jun. 12, 2015 | Zaire ebolavirus isolate Ebola virus/H.sapiens- |
| | | | | wt/GIN/2014/Makona-EM_078683, partial |
| | | | | genome |
| AKI83572 | Guinea | Dec. 14, 2014 | Jun. 12, 2015 | Zaire ebolavirus isolate Ebola virus/H.sapiens- |
| | | | | wt/GIN/2014/Makona-EM_078694, partial |
| | | | | genome |
| AKI83581 | Guinea | Dec. 14, 2014 | Jun. 12, 2015 | Zaire ebolavirus isolate Ebola virus/H.sapiens- |
| | | | | wt/GIN/2014/Makona-EM_078697, partial |
| | | | | genome |
| AKI83590 | Guinea | Dec. 15, 2014 | Jun. 12, 2015 | Zaire ebolavirus isolate Ebola virus/H.sapiens- |
| | | | | wt/GIN/2014/Makona-EM_078706, partial |
| | | | | genome |
| AKI83599 | Guinea | Dec. 14, 2014 | Jun. 12, 2015 | Zaire ebolavirus isolate Ebola virus/H.sapiens- |
| | | | | wt/GIN/2014/Makona-EM_078709, partial |
| | | | | genome |
| AKI83608 | Guinea | Dec. 16, 2014 | Jun. 12, 2015 | Zaire ebolavirus isolate Ebola virus/H.sapiens- |
| | | | | wt/GIN/2014/Makona-EM_078722, partial |
| | | | | genome |
| AKI83617 | Guinea | Dec. 17, 2014 | Jun. 12, 2015 | Zaire ebolavirus isolate Ebola virus/H.sapiens- |
| | | | | wt/GIN/2014/Makona-EM_078763, partial |
| | | | | genome |
| AKI83626 | Guinea | Dec. 18, 2014 | Jun. 12, 2015 | Zaire ebolavirus isolate Ebola virus/H.sapiens- |
| | | | | wt/GIN/2014/Makona-EM_078779, partial |
| | | | | genome |
| AKI83635 | Guinea | Jul. 30, 2014 | Jun. 12, 2015 | Zaire ebolavirus isolate Ebola virus/H.sapiens- |
| | | | | wt/GIN/2014/Makona-EM_079388, partial |
| | | | | genome |
| AKI83644 | Guinea | Mar. 28, 2014 | Jun. 12, 2015 | Zaire ebolavirus isolate Ebola virus/H.sapiens- |
| | | | | wt/GIN/2014/Makona-EM_079404, partial |
| | | | | genome |
| AKI83653 | Guinea | Mar. 28, 2014 | Jun. 12, 2015 | Zaire ebolavirus isolate Ebola virus/H.sapiens- |
| | | | | wt/GIN/2014/Makona-EM_079405, partial |
| | | | | genome |
| AKI83662 | Guinea | Mar. 31, 2014 | Jun. 12, 2015 | Zaire ebolavirus isolate Ebola virus/H.sapiens- |
| | | | | wt/GIN/2014/Makona-EM_079408, partial |
| | | | | genome |
| AKI83671 | Guinea | Mar. 31, 2014 | Jun. 12, 2015 | Zaire ebolavirus isolate Ebola virus/H.sapiens- |
| | | | | wt/GIN/2014/Makona-EM_079410, partial |
| | | | | genome |
| AKI83680 | Guinea | Mar. 31, 2014 | Jun. 12, 2015 | Zaire ebolavirus isolate Ebola virus/H.sapiens- |
| | | | | wt/GIN/2014/Makona-EM_079412, partial |
| | | | | genome |
| AKI83689 | Guinea | Mar. 31, 2014 | Jun. 12, 2015 | Zaire ebolavirus isolate Ebola virus/H.sapiens- |
| | | | | wt/GIN/2014/Makona-EM_079413, partial |
| | | | | genome |
| AKI83698 | Guinea | Mar. 31, 2014 | Jun. 12, 2015 | Zaire ebolavirus isolate Ebola virus/H.sapiens- |
| | | | | wt/GIN/2014/Makona-EM_079414, partial |
| | | | | genome |
| AKI83707 | Guinea | Mar. 30, 2014 | Jun. 12, 2015 | Zaire ebolavirus isolate Ebola virus/H.sapiens- |
| | | | | wt/GIN/2014/Makona-EM_079421, partial |
| | | | | genome |
| AKI83716 | Guinea | Mar. 27, 2014 | Jun. 12, 2015 | Zaire ebolavirus isolate Ebola virus/H.sapiens- |
| | | | | wt/GIN/2014/Makona-EM_079422, partial |
| | | | | genome |
| AKI83725 | Guinea | Mar. 27, 2014 | Jun. 12, 2015 | Zaire ebolavirus isolate Ebola virus/H.sapiens- |
| | | | | wt/GIN/2014/Makona-EM_079423, partial |
| | | | | genome |
| AKI83734 | Guinea | Mar. 27, 2014 | Jun. 12, 2015 | Zaire ebolavirus isolate Ebola virus/H.sapiens- |
| | | | | wt/GIN/2014/Makona-EM_079424, partial |
| | | | | genome |
| AKI83743 | Guinea | Apr. 2, 2014 | Jun. 12, 2015 | Zaire ebolavirus isolate Ebola virus/H.sapiens- |
| | | | | wt/GIN/2014/Makona-EM_079429, partial |
| | | | | genome |
| AKI83752 | Guinea | Apr. 2, 2014 | Jun. 12, 2015 | Zaire ebolavirus isolate Ebola virus/H.sapiens- |
| | | | | wt/GIN/2014/Makona-EM_079434, partial |
| | | | | genome |
| AKI83761 | Guinea | Apr. 3, 2014 | Jun. 12, 2015 | Zaire ebolavirus isolate Ebola virus/H.sapiens- |
| | | | | wt/GIN/2014/Makona-EM_079442, partial |
| | | | | genome |
| AKI83770 | Guinea | Apr. 2, 2014 | Jun. 12, 2015 | Zaire ebolavirus isolate Ebola virus/H.sapiens- |
| | | | | wt/GIN/2014/Makona-EM_079444, partial |
| | | | | genome |
| AKI83788 | Guinea | Apr. 4, 2014 | Jun. 12, 2015 | Zaire ebolavirus isolate Ebola virus/H.sapiens- |
| | | | | wt/GIN/2014/Makona-EM_079464, partial |
| | | | | genome |
| AKI83797 | Guinea | Apr. 7, 2014 | Jun. 12, 2015 | Zaire ebolavirus isolate Ebola virus/H.sapiens- |
| | | | | wt/GIN/2014/Makona-EM_079497, partial |
| | | | | genome |
| AKI83806 | Guinea | Apr. 10, 2014 | Jun. 12, 2015 | Zaire ebolavirus isolate Ebola virus/H.sapiens- |
| | | | | wt/GIN/2014/Makona-EM_079514, partial |
| | | | | genome |
| AKI83815 | Guinea | Apr. 11, 2014 | Jun. 12, 2015 | Zaire ebolavirus isolate Ebola virus/H.sapiens- |
| | | | | wt/GIN/2014/Makona-EM_079517, partial |
| | | | | genome |
| AKI83824 | Guinea | Apr. 12, 2014 | Jun. 12, 2015 | Zaire ebolavirus isolate Ebola virus/H.sapiens- |
| | | | | wt/GIN/2014/Makona-EM_079542, partial |
| | | | | genome |
| AKI83833 | Guinea | Apr. 13, 2014 | Jun. 12, 2015 | Zaire ebolavirus isolate Ebola virus/H.sapiens- |
| | | | | wt/GIN/2014/Makona-EM_079549, partial |
| | | | | genome |
| AKI83842 | Guinea | Apr. 18, 2014 | Jun. 12, 2015 | Zaire ebolavirus isolate Ebola virus/H.sapiens- |
| | | | | wt/GIN/2014/Makona-EM_079578, partial |
| | | | | genome |
| AKI83851 | Guinea | Apr. 22, 2014 | Jun. 12, 2015 | Zaire ebolavirus isolate Ebola virus/H.sapiens- |
| | | | | wt/GIN/2014/Makona-EM_079587, partial |
| | | | | genome |
| AKI83860 | Guinea | Apr. 28, 2014 | Jun. 12, 2015 | Zaire ebolavirus isolate Ebola virus/H.sapiens- |
| | | | | wt/GIN/2014/Makona-EM_079622, partial |
| | | | | genome |
| AKI83869 | Guinea | May 1, 2014 | Jun. 12, 2015 | Zaire ebolavirus isolate Ebola virus/H.sapiens- |
| | | | | wt/GIN/2014/Makona-EM_079630, partial |
| | | | | genome |
| AKI83878 | Guinea | May 7, 2014 | Jun. 12, 2015 | Zaire ebolavirus isolate Ebola virus/H.sapiens- |
| | | | | wt/GIN/2014/Makona-EM_079657, partial |
| | | | | genome |
| AKI83887 | Guinea | May 7, 2014 | Jun. 12, 2015 | Zaire ebolavirus isolate Ebola virus/H.sapiens- |
| | | | | wt/GIN/2014/Makona-EM_079659, partial |
| | | | | genome |
| AKI83896 | Guinea | May 10, 2014 | Jun. 12, 2015 | Zaire ebolavirus isolate Ebola virus/H.sapiens- |
| | | | | wt/GIN/2014/Makona-EM_079677, partial |
| | | | | genome |
| AKI83905 | Guinea | May 11, 2014 | Jun. 12, 2015 | Zaire ebolavirus isolate Ebola virus/H.sapiens- |
| | | | | wt/GIN/2014/Makona-EM_079681, partial |
| | | | | genome |
| AKI83914 | Guinea | May 11, 2014 | Jun. 12, 2015 | Zaire ebolavirus isolate Ebola virus/H.sapiens- |
| | | | | wt/GIN/2014/Makona-EM_079685, partial |
| | | | | genome |
| AKI83923 | Guinea | May 14, 2014 | Jun. 12, 2015 | Zaire ebolavirus isolate Ebola virus/H.sapiens- |
| | | | | wt/GIN/2014/Makona-EM_079702, partial |
| | | | | genome |
| AKI83932 | Guinea | May 18, 2014 | Jun. 12, 2015 | Zaire ebolavirus isolate Ebola virus/H.sapiens- |
| | | | | wt/GIN/2014/Makona-EM_079731, partial |
| | | | | genome |
| AKI83941 | Guinea | May 21, 2014 | Jun. 12, 2015 | Zaire ebolavirus isolate Ebola virus/H.sapiens- |
| | | | | wt/GIN/2014/Makona-EM_079749, partial |
| | | | | genome |
| AKI83950 | Guinea | May 21, 2014 | Jun. 12, 2015 | Zaire ebolavirus isolate Ebola virus/H.sapiens- |
| | | | | wt/GIN/2014/Makona-EM_079750, partial |
| | | | | genome |
| AKI83959 | Guinea | May 22, 2014 | Jun. 12, 2015 | Zaire ebolavirus isolate Ebola virus/H.sapiens- |
| | | | | wt/GIN/2014/Makona-EM_079753, partial |
| | | | | genome |
| AKI83968 | Guinea | May 24, 2014 | Jun. 12, 2015 | Zaire ebolavirus isolate Ebola virus/H.sapiens- |
| | | | | wt/GIN/2014/Makona-EM_079772, partial |
| | | | | genome |
| AKI83977 | Guinea | May 24, 2014 | Jun. 12, 2015 | Zaire ebolavirus isolate Ebola virus/H.sapiens- |
| | | | | wt/GIN/2014/Makona-EM_079775, partial |
| | | | | genome |
| AKI83986 | Guinea | May 28, 2014 | Jun. 12, 2015 | Zaire ebolavirus isolate Ebola virus/H.sapiens- |
| | | | | wt/GIN/2014/Makona-EM_079815, partial |
| | | | | genome |
| AKI83995 | Guinea | Jun. 1, 2014 | Jun. 12, 2015 | Zaire ebolavirus isolate Ebola virus/H.sapiens- |
| | | | | wt/GIN/2014/Makona-EM_079859, partial |
| | | | | genome |
| AKI84004 | Guinea | Jun. 5, 2014 | Jun. 12, 2015 | Zaire ebolavirus isolate Ebola virus/H.sapiens- |
| | | | | wt/GIN/2014/Makona-EM_079876, partial |
| | | | | genome |
| AKI84013 | Guinea | Jun. 5, 2014 | Jun. 12, 2015 | Zaire ebolavirus isolate Ebola virus/H.sapiens- |
| | | | | wt/GIN/2014/Makona-EM_079880, partial |
| | | | | genome |
| AKI84022 | Guinea | Jun. 9, 2014 | Jun. 12, 2015 | Zaire ebolavirus isolate Ebola virus/H.sapiens- |
| | | | | wt/GIN/2014/Makona-EM_079910, partial |
| | | | | genome |
| AKI84031 | Guinea | Jun. 9, 2014 | Jun. 12, 2015 | Zaire ebolavirus isolate Ebola virus/H.sapiens- |
| | | | | wt/GIN/2014/Makona-EM_079911, partial |
| | | | | genome |
| AKI84040 | Guinea | Jun. 9, 2014 | Jun. 12, 2015 | Zaire ebolavirus isolate Ebola virus/H.sapiens- |
| | | | | wt/GIN/2014/Makona-EM_079912, partial |
| | | | | genome |
| AKI84049 | Guinea | Jun. 9, 2014 | Jun. 12, 2015 | Zaire ebolavirus isolate Ebola virus/H.sapiens- |
| | | | | wt/GIN/2014/Makona-EM_079913, partial |
| | | | | genome |
| AKI84058 | Guinea | Jun. 10, 2014 | Jun. 12, 2015 | Zaire ebolavirus isolate Ebola virus/H.sapiens- |
| | | | | wt/GIN/2014/Makona-EM_079914, partial |
| | | | | genome |
| AKI84067 | Guinea | Jun. 10, 2014 | Jun. 12, 2015 | Zaire ebolavirus isolate Ebola virus/H.sapiens- |
| | | | | wt/GIN/2014/Makona-EM_079915, partial |
| | | | | genome |
| AKI84103 | Guinea | Jun. 20, 2014 | Jun. 12, 2015 | Zaire ebolavirus isolate Ebola virus/H.sapiens- |
| | | | | wt/GIN/2014/Makona-EM_080063, partial |
| | | | | genome |
| AKI84148 | Guinea | Jun. 22, 2014 | Jun. 12, 2015 | Zaire ebolavirus isolate Ebola virus/H.sapiens- |
| | | | | wt/GIN/2014/Makona-EM_080076, partial |
| | | | | genome |
| AKI84166 | Guinea | Jun. 25, 2014 | Jun. 12, 2015 | Zaire ebolavirus isolate Ebola virus/H.sapiens- |
| | | | | wt/GIN/2014/Makona-EM_080141, partial |
| | | | | genome |
| AKI84211 | Guinea | Jul. 10, 2014 | Jun. 12, 2015 | Zaire ebolavirus isolate Ebola virus/H.sapiens- |
| | | | | wt/GIN/2014/Makona-EM_080253, partial |
| | | | | genome |
| AKG65314 | Guinea | Sep. 21, 2014 | Jun. 26, 2015 | Zaire ebolavirus isolate Ebola virus/H.sapiens- |
| | | | | wt/GIN/2014/Makona-Forecariah-1069, partial |
| | | | | genome |
| AKG65521 | Guinea | Oct. 9, 2014 | Jun. 26, 2015 | Zaire ebolavirus isolate Ebola virus/H.sapiens- |
| | | | | wt/GIN/2014/Makona-Forecariah-1365, partial |
| | | | | genome |
| AKG65611 | Guinea | Oct. 18, 2014 | Jun. 26, 2015 | Zaire ebolavirus isolate Ebola virus/H.sapiens- |
| | | | | wt/GIN/2014/Makona-Forecariah-1567, partial |
| | | | | genome |
| AKG65620 | Guinea | Oct. 18, 2014 | Jun. 26, 2015 | Zaire ebolavirus isolate Ebola virus/H.sapiens- |
| | | | | wt/GIN/2014/Makona-Forecariah-1568, partial |
| | | | | genome |
| AKG65629 | Guinea | Oct. 20, 2014 | Jun. 26, 2015 | Zaire ebolavirus isolate Ebola virus/H.sapiens- |
| | | | | wt/GIN/2014/Makona-Forecariah-1571, partial |
| | | | | genome |
| AKG65647 | Guinea | Oct. 24, 2014 | Jun. 26, 2015 | Zaire ebolavirus isolate Ebola virus/H.sapiens- |
| | | | | wt/GIN/2014/Makona-Forecariah-1623, partial |
| | | | | genome |
| AKG65269 | Guinea | Sep. 15, 2014 | Jun. 26, 2015 | Zaire ebolavirus isolate Ebola virus/H.sapiens- |
| | | | | wt/GIN/2014/Makona-Forecariah-989, partial |
| | | | | genome |
| AKG65143 | Guinea | Aug. 12, 2014 | Jun. 26, 2015 | Zaire ebolavirus isolate Ebola virus/H.sapiens- |
| | | | | wt/GIN/2014/Makona-Gueckedou-633, partial |
| | | | | genome |
| AKG65467 | Guinea | Oct. 7, 2014 | Jun. 26, 2015 | Zaire ebolavirus isolate Ebola virus/H.sapiens- |
| | | | | wt/GIN/2014/Makona-Kerouane-1331, partial |
| | | | | genome |
| AKG65287 | Guinea | Sep. 20, 2014 | Jun. 26, 2015 | Zaire ebolavirus isolate Ebola virus/H.sapiens- |
| | | | | wt/GIN/2014/Makona-Kindia-1047, partial |
| | | | | genome |
| AKG65656 | Guinea | Oct. 23, 2014 | Jun. 26, 2015 | Zaire ebolavirus isolate Ebola virus/H.sapiens- |
| | | | | wt/GIN/2014/Makona-Kindia-1648, partial |
| | | | | genome |
| ALF04584 | Guinea | Dec. 18, 2014 | Sep. 26, 2015 | Zaire ebolavirus isolate Ebola virus/H.sapiens- |
| | | | | wt/GIN/2014/Makona-Kindia-802, partial genome |
| AKG65125 | Guinea | Jul. 27, 2014 | Jun. 26, 2015 | Zaire ebolavirus isolate Ebola virus/H.sapiens- |
| | | | | wt/GIN/2014/Makona-Kouroussa-531, partial |
| | | | | genome |
| AKG65152 | Guinea | Aug. 14, 2014 | Jun. 26, 2015 | Zaire ebolavirus isolate Ebola virus/H.sapiens- |
| | | | | wt/GIN/2014/Makona-Macenta-645, partial |
| | | | | genome |
| AKG65638 | Guinea | Oct. 22, 2014 | Jun. 26, 2015 | Zaire ebolavirus isolate Ebola virus/H.sapiens- |
| | | | | wt/GIN/2014/Makona-Nzeerekore-1622, partial |
| | | | | genome |
| AKG65116 | Guinea | Jul. 24, 2014 | Jun. 26, 2015 | Zaire ebolavirus isolate Ebola virus/H.sapiens- |
| | | | | wt/GIN/2014/Makona-Siguiri-517, partial genome |
| AKI82897 | Guinea | Jan. 2, 2015 | Jun. 12, 2015 | Zaire ebolavirus isolate Ebola virus/H.sapiens- |
| | | | | wt/GIN/2015/Makona-EM_004414, partial |
| | | | | genome |
| AKI82906 | Guinea | Jan. 2, 2015 | Jun. 12, 2015 | Zaire ebolavirus isolate Ebola virus/H.sapiens- |
| | | | | wt/GIN/2015/Makona-EM_004422, partial |
| | | | | genome |
| AKI82915 | Guinea | Jan. 4, 2015 | Jun. 12, 2015 | Zaire ebolavirus isolate Ebola virus/H.sapiens- |
| | | | | wt/GIN/2015/Makona-EM_004437, partial |
| | | | | genome |
| AKI82924 | Guinea | Jan. 4, 2015 | Jun. 12, 2015 | Zaire ebolavirus isolate Ebola virus/H.sapiens- |
| | | | | wt/GIN/2015/Makona-EM_004438, partial |
| | | | | genome |
| AKI82933 | Guinea | Jan. 11, 2015 | Jun. 12, 2015 | Zaire ebolavirus isolate Ebola virus/H.sapiens- |
| | | | | wt/GIN/2015/Makona-EM_004481, partial |
| | | | | genome |
| AKI82942 | Guinea | Jan. 14, 2015 | Jun. 12, 2015 | Zaire ebolavirus isolate Ebola virus/H.sapiens- |
| | | | | wt/GIN/2015/Makona-EM_004494, partial |
| | | | | genome |
| AKI82951 | Guinea | Jan. 15, 2015 | Jun. 12, 2015 | Zaire ebolavirus isolate Ebola virus/H.sapiens- |
| | | | | wt/GIN/2015/Makona-EM_004503, partial |
| | | | | genome |
| AKI82960 | Guinea | Jan. 22, 2015 | Jun. 12, 2015 | Zaire ebolavirus isolate Ebola virus/H.sapiens- |
| | | | | wt/GIN/2015/Makona-EM_004555, partial |
| | | | | genome |
| AKI82969 | Guinea | Jan. 25, 2015 | Jun. 12, 2015 | Zaire ebolavirus isolate Ebola virus/H.sapiens- |
| | | | | wt/GIN/2015/Makona-EM_004563, partial |
| | | | | genome |
| AKI82978 | Guinea | Jan. 27, 2015 | Jun. 12, 2015 | Zaire ebolavirus isolate Ebola virus/H.sapiens- |
| | | | | wt/GIN/2015/Makona-EM_004580, partial |
| | | | | genome |
| AKI82987 | Guinea | Jan. 31, 2015 | Jun. 12, 2015 | Zaire ebolavirus isolate Ebola virus/H.sapiens- |
| | | | | wt/GIN/2015/Makona-EM_004589, partial |
| | | | | genome |
| AJZ74730 | Liberia | Sep. 23, 2014 | Mar. 30, 2015 | Zaire ebolavirus isolate Ebola virus/H.sapiens- |
| | | | | wt/LBR/2014/Makona-10054, partial genome |
| AIY27577 | Liberia | Aug. 3, 2014 | Nov. 26, 2014 | Zaire ebolavirus isolate Ebola virus/H.sapiens- |
| | | | | wt/LBR/2014/Makona-201403007, complete |
| | | | | genome |
| AKI83005 | Liberia | Jul. 22, 2014 | Jun. 12, 2015 | Zaire ebolavirus isolate Ebola virus/H.sapiens- |
| | | | | wt/LBR/2014/Makona-EM_074349, partial |
| | | | | genome |
| AKI83014 | Liberia | Jul. 22, 2014 | Jun. 12, 2015 | Zaire ebolavirus isolate Ebola virus/H.sapiens- |
| | | | | wt/LBR/2014/Makona-EM_074350, partial |
| | | | | genome |
| AKI83023 | Liberia | Jul. 22, 2014 | Jun. 12, 2015 | Zaire ebolavirus isolate Ebola virus/H.sapiens- |
| | | | | wt/LBR/2014/Makona-EM_074351, partial |
| | | | | genome |
| AKI83032 | Liberia | Jul. 22, 2014 | Jun. 12, 2015 | Zaire ebolavirus isolate Ebola virus/H.sapiens- |
| | | | | wt/LBR/2014/Makona-EM_074352, partial |
| | | | | genome |
| AKI83041 | Liberia | Jul. 22, 2014 | Jun. 12, 2015 | Zaire ebolavirus isolate Ebola virus/H.sapiens- |
| | | | | wt/LBR/2014/Makona-EM_074353, partial |
| | | | | genome |
| AKI83059 | Liberia | Jul. 26, 2014 | Jun. 12, 2015 | Zaire ebolavirus isolate Ebola virus/H.sapiens- |
| | | | | wt/LBR/2014/Makona-EM_074391, partial |
| | | | | genome |
| AKI83068 | Liberia | Jul. 25, 2014 | Jun. 12, 2015 | Zaire ebolavirus isolate Ebola virus/H.sapiens- |
| | | | | wt/LBR/2014/Makona-EM_074392, partial |
| | | | | genome |
| AKI83140 | Liberia | Aug. 8, 2014 | Jun. 12, 2015 | Zaire ebolavirus isolate Ebola virus/H.sapiens- |
| | | | | wt/LBR/2014/Makona-EM_074548, partial |
| | | | | genome |
| AKI83158 | Liberia | Aug. 14, 2014 | Jun. 12, 2015 | Zaire ebolavirus isolate Ebola virus/H.sapiens- |
| | | | | wt/LBR/2014/Makona-EM_074720, partial |
| | | | | genome |
| AKI83176 | Liberia | Aug. 17, 2014 | Jun. 12, 2015 | Zaire ebolavirus isolate Ebola virus/H.sapiens- |
| | | | | wt/LBR/2014/Makona-EM_074821, partial |
| | | | | genome |
| AKI83185 | Liberia | Aug. 17, 2014 | Jun. 12, 2015 | Zaire ebolavirus isolate Ebola virus/H.sapiens- |
| | | | | wt/LBR/2014/Makona-EM_074822, partial |
| | | | | genome |
| AKI83194 | Liberia | Aug. 22, 2014 | Jun. 12, 2015 | Zaire ebolavirus isolate Ebola virus/H.sapiens- |
| | | | | wt/LBR/2014/Makona-EM_075043, partial |
| | | | | genome |
| AKI83779 | Liberia | Apr. 1, 2014 | Jun. 12, 2015 | Zaire ebolavirus isolate Ebola virus/H.sapiens- |
| | | | | wt/LBR/2014/Makona-EM_079450, partial |
| | | | | genome |
| AKI84112 | Liberia | Jun. 20, 2014 | Jun. 12, 2015 | Zaire ebolavirus isolate Ebola virus/H.sapiens- |
| | | | | wt/LBR/2014/Makona-EM_080064, partial |
| | | | | genome |
| AKI84121 | Liberia | Jun. 20, 2014 | Jun. 12, 2015 | Zaire ebolavirus isolate Ebola virus/H.sapiens- |
| | | | | wt/LBR/2014/Makona-EM_080065, partial |
| | | | | genome |
| AKI84130 | Liberia | Jun. 20, 2014 | Jun. 12, 2015 | Zaire ebolavirus isolate Ebola virus/H.sapiens- |
| | | | | wt/LBR/2014/Makona-EM_080066, partial |
| | | | | genome |
| AKI84139 | Liberia | Jun. 20, 2014 | Jun. 12, 2015 | Zaire ebolavirus isolate Ebola virus/H.sapiens- |
| | | | | wt/LBR/2014/Makona-EM_080067, partial |
| | | | | genome |
| AKI84184 | Liberia | Jun. 29, 2014 | Jun. 12, 2015 | Zaire ebolavirus isolate Ebola virus/H.sapiens- |
| | | | | wt/LBR/2014/Makona-EM_080193, partial |
| | | | | genome |
| AKI84193 | Liberia | Jul. 3, 2014 | Jun. 12, 2015 | Zaire ebolavirus isolate Ebola virus/H.sapiens- |
| | | | | wt/LBR/2014/Makona-EM_080213, partial |
| | | | | genome |
| AKI84202 | Liberia | Jul. 4, 2014 | Jun. 12, 2015 | Zaire ebolavirus isolate Ebola virus/H.sapiens- |
| | | | | wt/LBR/2014/Makona-EM_080223, partial |
| | | | | genome |
| AKI84220 | Liberia | Jul. 12, 2014 | Jun. 12, 2015 | Zaire ebolavirus isolate Ebola virus/H.sapiens- |
| | | | | wt/LBR/2014/Makona-EM_080261, partial |
| | | | | genome |
| AKI84238 | Liberia | Jul. 12, 2014 | Jun. 12, 2015 | Zaire ebolavirus isolate Ebola virus/H.sapiens- |
| | | | | wt/LBR/2014/Makona-EM_080269, partial |
| | | | | genome |
| AJZ74520 | Liberia | Nov. 5, 2014 | Mar. 30, 2015 | Zaire ebolavirus isolate Ebola virus/H.sapiens- |
| | | | | wt/LBR/2014/Makona-LIBR0058, partial genome |
| AJZ74547 | Liberia | Nov. 6, 2014 | Mar. 30, 2015 | Zaire ebolavirus isolate Ebola virus/H.sapiens- |
| | | | | wt/LBR/2014/Makona-LIBR0067, partial genome |
| AJZ74556 | Liberia | Nov. 6, 2014 | Mar. 30, 2015 | Zaire ebolavirus isolate Ebola virus/H.sapiens- |
| | | | | wt/LBR/2014/Makona-LIBR0073, partial genome |
| AJZ74565 | Liberia | Nov. 8, 2014 | Mar. 30, 2015 | Zaire ebolavirus isolate Ebola virus/H.sapiens- |
| | | | | wt/LBR/2014/Makona-LIBR0090, partial genome |
| AJZ74574 | Liberia | Nov. 8, 2014 | Mar. 30, 2015 | Zaire ebolavirus isolate Ebola virus/H.sapiens- |
| | | | | wt/LBR/2014/Makona-LIBR0092, partial genome |
| AJZ74583 | Liberia | Nov. 8, 2014 | Mar. 30, 2015 | Zaire ebolavirus isolate Ebola virus/H.sapiens- |
| | | | | wt/LBR/2014/Makona-LIBR0093, partial genome |
| AJZ74592 | Liberia | Nov. 10, 2014 | Mar. 30, 2015 | Zaire ebolavirus isolate Ebola virus/H.sapiens- |
| | | | | wt/LBR/2014/Makona-LIBR0116, partial genome |
| AJZ74601 | Liberia | Nov. 13, 2014 | Mar. 30, 2015 | Zaire ebolavirus isolate Ebola virus/H.sapiens- |
| | | | | wt/LBR/2014/Makona-LIBR0168, partial genome |
| AJZ74626 | Liberia | Nov. 22, 2014 | Mar. 30, 2015 | Zaire ebolavirus isolate Ebola virus/H.sapiens- |
| | | | | wt/LBR/2014/Makona-LIBR0286, partial genome |
| AJZ74635 | Liberia | Nov. 25, 2014 | Mar. 30, 2015 | Zaire ebolavirus isolate Ebola virus/H.sapiens- |
| | | | | wt/LBR/2014/Makona-LIBR0333, partial genome |
| AJZ74644 | Liberia | Dec. 3, 2014 | Mar. 30, 2015 | Zaire ebolavirus isolate Ebola virus/H.sapiens- |
| | | | | wt/LBR/2014/Makona-LIBR0423, partial genome |
| AJZ74660 | Liberia | Dec. 10, 2014 | Mar. 30, 2015 | Zaire ebolavirus isolate Ebola virus/H.sapiens- |
| | | | | wt/LBR/2014/Makona-LIBR0503, partial genome |
| AJZ74669 | Liberia | Dec. 10, 2014 | Mar. 30, 2015 | Zaire ebolavirus isolate Ebola virus/H.sapiens- |
| | | | | wt/LBR/2014/Makona-LIBR0505, partial genome |
| AJZ74721 | Liberia | Oct. 1, 2014 | Mar. 30, 2015 | Zaire ebolavirus isolate Ebola virus/H.sapiens- |
| | | | | wt/LBR/2014/Makona-LIBR10053, partial |
| | | | | genome |
| AJZ74695 | Liberia | Jan. 20, 2015 | Mar. 30, 2015 | Zaire ebolavirus isolate Ebola virus/H.sapiens- |
| | | | | wt/LBR/2015/Makona-LIBR0993, partial genome |
| AJZ74712 | Liberia | Feb. 14, 2015 | Mar. 30, 2015 | Zaire ebolavirus isolate Ebola virus/H.sapiens- |
| | | | | wt/LBR/2015/Makona-LIBR1413, partial genome |
| AIZ68608 | Mali | Oct. 23, 2014 | Dec. 12, 2014 | Zaire ebolavirus isolate Ebola virus/H.sapiens- |
| | | | | wt/MLI/2014/Makona-Mali-DPR1, complete |
| | | | | genome |
| AIZ68616 | Mali | Nov. 12, 2014 | Dec. 12, 2014 | Zaire ebolavirus isolate Ebola virus/H.sapiens- |
| | | | | wt/MLI/2014/Makona-Mali-DPR2, complete |
| | | | | genome |
| AIZ68624 | Mali | Nov. 21, 2014 | Dec. 12, 2014 | Zaire ebolavirus isolate Ebola virus/H.sapiens- |
| | | | | wt/MLI/2014/Makona-Mali-DPR3, complete |
| | | | | genome |
| AIZ68632 | Mali | Nov. 12, 2014 | Dec. 12, 2014 | Zaire ebolavirus isolate Ebola virus/H.sapiens- |
| | | | | wt/MLI/2014/Makona-Mali-DPR4, complete |
| | | | | genome |
| AKG95786 | Sierra | Aug. 22, 2014 | May 17, 2015 | Zaire ebolavirus isolate Ebola virus/H.sapiens- |
| | Leone | | | wt/SLE/2014/Makona-20140008, partial genome |
| AKG95795 | Sierra | Aug. 20, 2014 | May 17, 2015 | Zaire ebolavirus isolate Ebola virus/H.sapiens- |
| | Leone | | | wt/SLE/2014/Makona-20140024, partial genome |
| AKG95930 | Sierra | Aug. 23, 2014 | May 17, 2015 | Zaire ebolavirus isolate Ebola virus/H.sapiens- |
| | Leone | | | wt/SLE/2014/Makona-20140038, partial genome |
| AKG95678 | Sierra | Aug. 22, 2014 | May 17, 2015 | Zaire ebolavirus isolate Ebola virus/H.sapiens- |
| | Leone | | | wt/SLE/2014/Makona-20140091, partial genome |
| AKG95696 | Sierra | Aug. 24, 2014 | May 17, 2015 | Zaire ebolavirus isolate Ebola virus/H.sapiens- |
| | Leone | | | wt/SLE/2014/Makona-20140100, partial genome |
| AKG95570 | Sierra | Aug. 26, 2014 | May 17, 2015 | Zaire ebolavirus isolate Ebola virus/H.sapiens- |
| | Leone | | | wt/SLE/2014/Makona-20140134, partial genome |
| AKG95912 | Sierra | Aug. 27, 2014 | May 17, 2015 | Zaire ebolavirus isolate Ebola virus/H.sapiens- |
| | Leone | | | wt/SLE/2014/Makona-20140161, partial genome |
| AKG96173 | Sierra | Aug. 27, 2014 | May 17, 2015 | Zaire ebolavirus isolate Ebola virus/H.sapiens- |
| | Leone | | | wt/SLE/2014/Makona-20140174, partial genome |
| AKG96191 | Sierra | Aug. 29, 2014 | May 17, 2015 | Zaire ebolavirus isolate Ebola virus/H.sapiens- |
| | Leone | | | wt/SLE/2014/Makona-20140254, partial genome |
| AKG96038 | Sierra | Sep. 2, 2014 | May 17, 2015 | Zaire ebolavirus isolate Ebola virus/H.sapiens- |
| | Leone | | | wt/SLE/2014/Makona-20140395, partial genome |
| AKG95741 | Sierra | Sep. 3, 2014 | May 17, 2015 | Zaire ebolavirus isolate Ebola virus/H.sapiens- |
| | Leone | | | wt/SLE/2014/Makona-20140433, partial genome |
| AKG96110 | Sierra | Sep. 3, 2014 | May 17, 2015 | Zaire ebolavirus isolate Ebola virus/H.sapiens- |
| | Leone | | | wt/SLE/2014/Makona-20140436, partial genome |
| AKG95642 | Sierra | Sep. 4, 2014 | May 17, 2015 | Zaire ebolavirus isolate Ebola virus/H.sapiens- |
| | Leone | | | wt/SLE/2014/Makona-20140489, partial genome |
| AKG95894 | Sierra | Sep. 5, 2014 | May 17, 2015 | Zaire ebolavirus isolate Ebola virus/H.sapiens- |
| | Leone | | | wt/SLE/2014/Makona-20140517, partial genome |
| AKG96029 | Sierra | Sep. 7, 2014 | May 17, 2015 | Zaire ebolavirus isolate Ebola virus/H.sapiens- |
| | Leone | | | wt/SLE/2014/Makona-20140590, partial genome |
| AKG96101 | Sierra | Sep. 10, 2014 | May 17, 2015 | Zaire ebolavirus isolate Ebola virus/H.sapiens- |
| | Leone | | | wt/SLE/2014/Makona-20140729, partial genome |
| AKG96200 | Sierra | Sep. 15, 2014 | May 17, 2015 | Zaire ebolavirus isolate Ebola virus/H.sapiens- |
| | Leone | | | wt/SLE/2014/Makona-20140872, partial genome |
| AKG95948 | Sierra | Sep. 18, 2014 | May 17, 2015 | Zaire ebolavirus isolate Ebola virus/H.sapiens- |
| | Leone | | | wt/SLE/2014/Makona-20140910, partial genome |
| AKG96047 | Sierra | Sep. 16, 2014 | May 17, 2015 | Zaire ebolavirus isolate Ebola virus/H.sapiens- |
| | Leone | | | wt/SLE/2014/Makona-20140933, partial genome |
| AKG95723 | Sierra | Sep. 21, 2014 | May 17, 2015 | Zaire ebolavirus isolate Ebola virus/H.sapiens- |
| | Leone | | | wt/SLE/2014/Makona-20141012, partial genome |
| AKG96272 | Sierra | Sep. 21, 2014 | May 17, 2015 | Zaire ebolavirus isolate Ebola virus/H.sapiens- |
| | Leone | | | wt/SLE/2014/Makona-20141043, partial genome |
| AKG95777 | Sierra | Sep. 21, 2014 | May 17, 2015 | Zaire ebolavirus isolate Ebola virus/H.sapiens- |
| | Leone | | | wt/SLE/2014/Makona-20141061, partial genome |
| AKG96146 | Sierra | Sep. 22, 2014 | May 17, 2015 | Zaire ebolavirus isolate Ebola virus/H.sapiens- |
| | Leone | | | wt/SLE/2014/Makona-20141123, partial genome |
| AKG96254 | Sierra | Sep. 26, 2014 | May 17, 2015 | Zaire ebolavirus isolate Ebola virus/H.sapiens- |
| | Leone | | | wt/SLE/2014/Makona-20141227, partial genome |
| AKG95921 | Sierra | Sep. 25, 2014 | May 17, 2015 | Zaire ebolavirus isolate Ebola virus/H.sapiens- |
| | Leone | | | wt/SLE/2014/Makona-20141232, partial genome |
| AKG96227 | Sierra | Sep. 24, 2014 | May 17, 2015 | Zaire ebolavirus isolate Ebola virus/H.sapiens- |
| | Leone | | | wt/SLE/2014/Makona-20141241, partial genome |
| AKG95876 | Sierra | Sep. 24, 2014 | May 17, 2015 | Zaire ebolavirus isolate Ebola virus/H.sapiens- |
| | Leone | | | wt/SLE/2014/Makona-20141271, partial genome |
| AKG96011 | Sierra | Sep. 23, 2014 | May 17, 2015 | Zaire ebolavirus isolate Ebola virus/H.sapiens- |
| | Leone | | | wt/SLE/2014/Makona-20141280, partial genome |
| AKG95588 | Sierra | Sep. 23, 2014 | May 17, 2015 | Zaire ebolavirus isolate Ebola virus/H.sapiens- |
| | Leone | | | wt/SLE/2014/Makona-20141282, partial genome |
| AKG95615 | Sierra | Sep. 23, 2014 | May 17, 2015 | Zaire ebolavirus isolate Ebola virus/H.sapiens- |
| | Leone | | | wt/SLE/2014/Makona-20141288, partial genome |
| AKG96083 | Sierra | Sep. 26, 2014 | May 17, 2015 | Zaire ebolavirus isolate Ebola virus/H.sapiens- |
| | Leone | | | wt/SLE/2014/Makona-20141352, partial genome |
| AKG96119 | Sierra | Sep. 28, 2014 | May 17, 2015 | Zaire ebolavirus isolate Ebola virus/H.sapiens- |
| | Leone | | | wt/SLE/2014/Makona-20141397, partial genome |
| AKG95867 | Sierra | Sep. 28, 2014 | May 17, 2015 | Zaire ebolavirus isolate Ebola virus/H.sapiens- |
| | Leone | | | wt/SLE/2014/Makona-20141429, partial genome |
| AKG96128 | Sierra | Oct. 1, 2014 | May 17, 2015 | Zaire ebolavirus isolate Ebola virus/H.sapiens- |
| | Leone | | | wt/SLE/2014/Makona-20141491, partial genome |
| AKG95984 | Sierra | Oct. 1, 2014 | May 17, 2015 | Zaire ebolavirus isolate Ebola virus/H.sapiens- |
| | Leone | | | wt/SLE/2014/Makona-20141497, partial genome |
| AKG95597 | Sierra | Oct. 3, 2014 | May 17, 2015 | Zaire ebolavirus isolate Ebola virus/H.sapiens- |
| | Leone | | | wt/SLE/2014/Makona-20141582, partial genome |
| AKG95624 | Sierra | Oct. 4, 2014 | May 17, 2015 | Zaire ebolavirus isolate Ebola virus/H.sapiens- |
| | Leone | | | wt/SLE/2014/Makona-20141643, partial genome |
| AKG95552 | Sierra | Oct. 5, 2014 | May 17, 2015 | Zaire ebolavirus isolate Ebola virus/H.sapiens- |
| | Leone | | | wt/SLE/2014/Makona-20141650, partial genome |
| AKG96074 | Sierra | Oct. 9, 2014 | May 17, 2015 | Zaire ebolavirus isolate Ebola virus/H.sapiens- |
| | Leone | | | wt/SLE/2014/Makona-20141960, partial genome |
| AKG96164 | Sierra | Oct. 10, 2014 | May 17, 2015 | Zaire ebolavirus isolate Ebola virus/H.sapiens- |
| | Leone | | | wt/SLE/2014/Makona-20141997, partial genome |
| AKG95993 | Sierra | Oct. 11, 2014 | May 17, 2015 | Zaire ebolavirus isolate Ebola virus/H.sapiens- |
| | Leone | | | wt/SLE/2014/Makona-20142065, partial genome |
| AKG95633 | Sierra | Oct. 12, 2014 | May 17, 2015 | Zaire ebolavirus isolate Ebola virus/H.sapiens- |
| | Leone | | | wt/SLE/2014/Makona-20142127, partial genome |
| AKG95885 | Sierra | Oct. 14, 2014 | May 17, 2015 | Zaire ebolavirus isolate Ebola virus/H.sapiens- |
| | Leone | | | wt/SLE/2014/Makona-20142260, partial genome |
| AKG95768 | Sierra | Oct. 16, 2014 | May 17, 2015 | Zaire ebolavirus isolate Ebola virus/H.sapiens- |
| | Leone | | | wt/SLE/2014/Makona-20142407, partial genome |
| AKG95813 | Sierra | Oct. 18, 2014 | May 17, 2015 | Zaire ebolavirus isolate Ebola virus/H.sapiens- |
| | Leone | | | wt/SLE/2014/Makona-20142417, partial genome |
| AKG95822 | Sierra | Oct. 19, 2014 | May 17, 2015 | Zaire ebolavirus isolate Ebola virus/H.sapiens- |
| | Leone | | | wt/SLE/2014/Makona-20142477, partial genome |
| AKG95561 | Sierra | Oct. 18, 2014 | May 17, 2015 | Zaire ebolavirus isolate Ebola virus/H.sapiens- |
| | Leone | | | wt/SLE/2014/Makona-20142551, partial genome |
| AKG96236 | Sierra | Oct. 23, 2014 | May 17, 2015 | Zaire ebolavirus isolate Ebola virus/H.sapiens- |
| | Leone | | | wt/SLE/2014/Makona-20142843, partial genome |
| AKG96245 | Sierra | Oct. 23, 2014 | May 17, 2015 | Zaire ebolavirus isolate Ebola virus/H.sapiens- |
| | Leone | | | wt/SLE/2014/Makona-20142856, partial genome |
| AKG95939 | Sierra | Oct. 24, 2014 | May 17, 2015 | Zaire ebolavirus isolate Ebola virus/H.sapiens- |
| | Leone | | | wt/SLE/2014/Makona-20142895, partial genome |
| AKG95975 | Sierra | Oct. 26, 2014 | May 17, 2015 | Zaire ebolavirus isolate Ebola virus/H.sapiens- |
| | Leone | | | wt/SLE/2014/Makona-20143018, partial genome |
| AKG96002 | Sierra | Oct. 25, 2014 | May 17, 2015 | Zaire ebolavirus isolate Ebola virus/H.sapiens- |
| | Leone | | | wt/SLE/2014/Makona-20143031, partial genome |
| AKG96092 | Sierra | Oct. 24, 2014 | May 17, 2015 | Zaire ebolavirus isolate Ebola virus/H.sapiens- |
| | Leone | | | wt/SLE/2014/Makona-20143036, partial genome |
| AKG95750 | Sierra | Oct. 25, 2014 | May 17, 2015 | Zaire ebolavirus isolate Ebola virus/H.sapiens- |
| | Leone | | | wt/SLE/2014/Makona-20143107, partial genome |
| AKG96020 | Sierra | Oct. 27, 2014 | May 17, 2015 | Zaire ebolavirus isolate Ebola virus/H.sapiens- |
| | Leone | | | wt/SLE/2014/Makona-20143164, partial genome |
| AKG96209 | Sierra | Oct. 28, 2014 | May 17, 2015 | Zaire ebolavirus isolate Ebola virus/H.sapiens- |
| | Leone | | | wt/SLE/2014/Makona-20143187, partial genome |
| AKG96065 | Sierra | Oct. 29, 2014 | May 17, 2015 | Zaire ebolavirus isolate Ebola virus/H.sapiens- |
| | Leone | | | wt/SLE/2014/Makona-20143317, partial genome |
| AKG95849 | Sierra | Oct. 30, 2014 | May 17, 2015 | Zaire ebolavirus isolate Ebola virus/H.sapiens- |
| | Leone | | | wt/SLE/2014/Makona-20143360, partial genome |
| AKG95759 | Sierra | Oct. 31, 2014 | May 17, 2015 | Zaire ebolavirus isolate Ebola virus/H.sapiens- |
| | Leone | | | wt/SLE/2014/Makona-20143415, partial genome |
| AKG95732 | Sierra | Nov. 1, 2014 | May 17, 2015 | Zaire ebolavirus isolate Ebola virus/H.sapiens- |
| | Leone | | | wt/SLE/2014/Makona-20143458, partial genome |
| AKG96263 | Sierra | Nov. 1, 2014 | May 17, 2015 | Zaire ebolavirus isolate Ebola virus/H.sapiens- |
| | Leone | | | wt/SLE/2014/Makona-20143466, partial genome |
| AKG95579 | Sierra | Nov. 1, 2014 | May 17, 2015 | Zaire ebolavirus isolate Ebola virus/H.sapiens- |
| | Leone | | | wt/SLE/2014/Makona-20143550, partial genome |
| AKG95804 | Sierra | Nov. 3, 2014 | May 17, 2015 | Zaire ebolavirus isolate Ebola virus/H.sapiens- |
| | Leone | | | wt/SLE/2014/Makona-20143648, partial genome |
| AKG95831 | Sierra | Nov. 3, 2014 | May 17, 2015 | Zaire ebolavirus isolate Ebola virus/H.sapiens- |
| | Leone | | | wt/SLE/2014/Makona-20143659, partial genome |
| AKG96155 | Sierra | Nov. 4, 2014 | May 17, 2015 | Zaire ebolavirus isolate Ebola virus/H.sapiens- |
| | Leone | | | wt/SLE/2014/Makona-20143716, partial genome |
| AKG95651 | Sierra | Nov. 5, 2014 | May 17, 2015 | Zaire ebolavirus isolate Ebola virus/H.sapiens- |
| | Leone | | | wt/SLE/2014/Makona-20143753, partial genome |
| AKG95840 | Sierra | Nov. 5, 2014 | May 17, 2015 | Zaire ebolavirus isolate Ebola virus/H.sapiens- |
| | Leone | | | wt/SLE/2014/Makona-20143796, partial genome |
| AKG95687 | Sierra | Nov. 6, 2014 | May 17, 2015 | Zaire ebolavirus isolate Ebola virus/H.sapiens- |
| | Leone | | | wt/SLE/2014/Makona-20143918, partial genome |
| AKG95903 | Sierra | Nov. 7, 2014 | May 17, 2015 | Zaire ebolavirus isolate Ebola virus/H.sapiens- |
| | Leone | | | wt/SLE/2014/Makona-20143938, partial genome |
| AKG95705 | Sierra | Nov. 7, 2014 | May 17, 2015 | Zaire ebolavirus isolate Ebola virus/H.sapiens- |
| | Leone | | | wt/SLE/2014/Makona-20143964, partial genome |
| AKG95669 | Sierra | Nov. 8, 2014 | May 17, 2015 | Zaire ebolavirus isolate Ebola virus/H.sapiens- |
| | Leone | | | wt/SLE/2014/Makona-20144192, partial genome |
| AKG95966 | Sierra | Nov. 12, 2014 | May 17, 2015 | Zaire ebolavirus isolate Ebola virus/H.sapiens- |
| | Leone | | | wt/SLE/2014/Makona-20144521, partial genome |
| AKG95957 | Sierra | Nov. 12, 2014 | May 17, 2015 | Zaire ebolavirus isolate Ebola virus/H.sapiens- |
| | Leone | | | wt/SLE/2014/Makona-20144610, partial genome |
| AKG96218 | Sierra | Nov. 15, 2014 | May 17, 2015 | Zaire ebolavirus isolate Ebola virus/H.sapiens- |
| | Leone | | | wt/SLE/2014/Makona-20144819, partial genome |
| AKG95714 | Sierra | Nov. 15, 2014 | May 17, 2015 | Zaire ebolavirus isolate Ebola virus/H.sapiens- |
| | Leone | | | wt/SLE/2014/Makona-20144820, partial genome |
| AKG95858 | Sierra | Nov. 14, 2014 | May 17, 2015 | Zaire ebolavirus isolate Ebola virus/H.sapiens- |
| | Leone | | | wt/SLE/2014/Makona-20144837, partial genome |
| AKG95606 | Sierra | Nov. 13, 2014 | May 17, 2015 | Zaire ebolavirus isolate Ebola virus/H.sapiens- |
| | Leone | | | wt/SLE/2014/Makona-20144865, partial genome |
| AKG96182 | Sierra | Nov. 25, 2014 | May 17, 2015 | Zaire ebolavirus isolate Ebola virus/H.sapiens- |
| | Leone | | | wt/SLE/2014/Makona-20145835, partial genome |
| AKG96137 | Sierra | Nov. 26, 2014 | May 17, 2015 | Zaire ebolavirus isolate Ebola virus/H.sapiens- |
| | Leone | | | wt/SLE/2014/Makona-20145853, partial genome |
| AKG95660 | Sierra | Nov. 24, 2014 | May 17, 2015 | Zaire ebolavirus isolate Ebola virus/H.sapiens- |
| | Leone | | | wt/SLE/2014/Makona-20146001, partial genome |
| AKG95543 | Sierra | Dec. 22, 2014 | May 17, 2015 | Zaire ebolavirus isolate Ebola virus/H.sapiens- |
| | Leone | | | wt/SLE/2014/Makona-20146553, partial genome |
| AKG96056 | Sierra | Dec. 26, 2014 | May 17, 2015 | Zaire ebolavirus isolate Ebola virus/H.sapiens- |
| | Leone | | | wt/SLE/2014/Makona-20146578, partial genome |
| AIE11810 | Sierra | May 25, 2014 | Jun. 30, 2014 | Zaire ebolavirus isolate Ebola virus/H.sapiens- |
| | Leone | | | wt/SLE/2014/Makona-EM095, complete genome |
| AIE11801 | Sierra | May 25, 2014 | Jun. 30, 2014 | Zaire ebolavirus isolate Ebola virus/H.sapiens- |
| | Leone | | | wt/SLE/2014/Makona-EM095B, complete |
| | | | | genome |
| AIE11819 | Sierra | May 26, 2014 | Jun. 30, 2014 | Zaire ebolavirus isolate Ebola virus/H.sapiens- |
| | Leone | | | wt/SLE/2014/Makona-EM096, complete genome |
| AIE11828 | Sierra | May 26, 2014 | Jun. 30, 2014 | Zaire ebolavirus isolate Ebola virus/H.sapiens- |
| | Leone | | | wt/SLE/2014/Makona-EM098, complete genome |
| AIG95888 | Sierra | Jun. 2, 2014 | Jul. 25, 2014 | Zaire ebolavirus isolate Ebola virus/H.sapiens- |
| | Leone | | | wt/SLE/2014/Makona-EM104, complete genome |
| AIG95897 | Sierra | Jun. 2, 2014 | Jul. 25, 2014 | Zaire ebolavirus isolate Ebola virus/H.sapiens- |
| | Leone | | | wt/SLE/2014/Makona-EM106, complete genome |
| AIG95906 | Sierra | Jun. 3, 2014 | Jul. 25, 2014 | Zaire ebolavirus isolate Ebola virus/H.sapiens- |
| | Leone | | | wt/SLE/2014/Makona-EM110, complete genome |
| AIG95915 | Sierra | Jun. 3, 2014 | Jul. 25, 2014 | Zaire ebolavirus isolate Ebola virus/H.sapiens- |
| | Leone | | | wt/SLE/2014/Makona-EM111, complete genome |
| AIG95924 | Sierra | Jun. 3, 2014 | Jul. 25, 2014 | Zaire ebolavirus isolate Ebola virus/H.sapiens- |
| | Leone | | | wt/SLE/2014/Makona-EM112, complete genome |
| AIG95933 | Sierra | Jun. 3, 2014 | Jul. 25, 2014 | Zaire ebolavirus isolate Ebola virus/H.sapiens- |
| | Leone | | | wt/SLE/2014/Makona-EM113, complete genome |
| AIG95942 | Sierra | Jun. 3, 2014 | Jul. 25, 2014 | Zaire ebolavirus isolate Ebola virus/H.sapiens- |
| | Leone | | | wt/SLE/2014/Makona-EM115, complete genome |
| AIG95951 | Sierra | Jun. 3, 2014 | Jul. 25, 2014 | Zaire ebolavirus isolate Ebola virus/H.sapiens- |
| | Leone | | | wt/SLE/2014/Makona-EM119, complete genome |
| AIG95960 | Sierra | Jun. 3, 2014 | Jul. 25, 2014 | Zaire ebolavirus isolate Ebola virus/H.sapiens- |
| | Leone | | | wt/SLE/2014/Makona-EM120, complete genome |
| AIG95969 | Sierra | Jun. 4, 2014 | Jul. 25, 2014 | Zaire ebolavirus isolate Ebola virus/H.sapiens- |
| | Leone | | | wt/SLE/2014/Makona-EM121, complete genome |
| AIG95978 | Sierra | Jun. 4, 2014 | Jul. 25, 2014 | Zaire ebolavirus isolate Ebola virus/H.sapiens- |
| | Leone | | | wt/SLE/2014/Makona-EM124.1, complete |
| | | | | genome |
| AIG95987 | Sierra | Jun. 6, 2014 | Jul. 25, 2014 | Zaire ebolavirus isolate Ebola virus/H.sapiens- |
| | Leone | | | wt/SLE/2014/Makona-EM124.2, complete |
| | | | | genome |
| AIG95996 | Sierra | Jun. 8, 2014 | Jul. 25, 2014 | Zaire ebolavirus isolate Ebola virus/H.sapiens- |
| | Leone | | | wt/SLE/2014/Makona-EM124.3, complete |
| | | | | genome |
| AIG96005 | Sierra | Jun. 9, 2014 | Jul. 25, 2014 | Zaire ebolavirus isolate Ebola virus/H.sapiens- |
| | Leone | | | wt/SLE/2014/Makona-EM124.4, complete |
| | | | | genome |
| AKI84076 | Sierra | Jun. 13, 2014 | Jun. 12, 2015 | Zaire ebolavirus isolate Ebola virus/H.sapiens- |
| | Leone | | | wt/SLE/2014/Makona-EM_079983, partial |
| | | | | genome |
| AKI84085 | Sierra | Jun. 14, 2014 | Jun. 12, 2015 | Zaire ebolavirus isolate Ebola virus/H.sapiens- |
| | Leone | | | wt/SLE/2014/Makona-EM_080003, partial |
| | | | | genome |
| AKI84094 | Sierra | Jun. 15, 2014 | Jun. 12, 2015 | Zaire ebolavirus isolate Ebola virus/H.sapiens- |
| | Leone | | | wt/SLE/2014/Makona-EM_080011, partial |
| | | | | genome |
| AKI84157 | Sierra | Jun. 24, 2014 | Jun. 12, 2015 | Zaire ebolavirus isolate Ebola virus/H.sapiens- |
| | Leone | | | wt/SLE/2014/Makona-EM_080132, partial |
| | | | | genome |
| AKI84175 | Sierra | Jun. 26, 2014 | Jun. 12, 2015 | Zaire ebolavirus isolate Ebola virus/H.sapiens- |
| | Leone | | | wt/SLE/2014/Makona-EM_080165, partial |
| | | | | genome |
| AKI84229 | Sierra | Jul. 12, 2014 | Jun. 12, 2015 | Zaire ebolavirus isolate Ebola virus/H.sapiens- |
| | Leone | | | wt/SLE/2014/Makona-EM_080265, partial |
| | | | | genome |
| AIE11837 | Sierra | May 27, 2014 | Jun. 30, 2014 | Zaire ebolavirus isolate Ebola virus/H.sapiens- |
| | Leone | | | wt/SLE/2014/Makona-G3670.1, complete |
| | | | | genome |
| AIE11846 | Sierra | May 27, 2014 | Jun. 30, 2014 | Zaire ebolavirus isolate Ebola virus/H.sapiens- |
| | Leone | | | wt/SLE/2014/Makona-G3676.1, complete |
| | | | | genome |
| AIE11855 | Sierra | Jun. 6, 2014 | Jun. 30, 2014 | Zaire ebolavirus isolate Ebola virus/H.sapiens- |
| | Leone | | | wt/SLE/2014/Makona-G3676.2, complete |
| | | | | genome |
| AIE11864 | Sierra | May 26, 2014 | Jun. 30, 2014 | Zaire ebolavirus isolate Ebola virus/H.sapiens- |
| | Leone | | | wt/SLE/2014/Makona-G3677.1, complete |
| | | | | genome |
| AIE11873 | Sierra | May 27, 2014 | Jun. 30, 2014 | Zaire ebolavirus isolate Ebola virus/H.sapiens- |
| | Leone | | | wt/SLE/2014/Makona-G3677.2, complete |
| | | | | genome |
| AIE11882 | Sierra | May 28, 2014 | Jun. 30, 2014 | Zaire ebolavirus isolate Ebola virus/H.sapiens- |
| | Leone | | | wt/SLE/2014/Makona-G3679.1, complete |
| | | | | genome |
| AIE11891 | Sierra | May 28, 2014 | Jun. 30, 2014 | Zaire ebolavirus isolate Ebola virus/H.sapiens- |
| | Leone | | | wt/SLE/2014/Makona-G3680.1, complete |
| | | | | genome |
| AIE11900 | Sierra | May 28, 2014 | Jun. 30, 2014 | Zaire ebolavirus isolate Ebola virus/H.sapiens- |
| | Leone | | | wt/SLE/2014/Makona-G3682.1, complete |
| | | | | genome |
| AIE11909 | Sierra | May 28, 2014 | Jun. 30, 2014 | Zaire ebolavirus isolate Ebola virus/H.sapiens- |
| | Leone | | | wt/SLE/2014/Makona-G3683.1, complete |
| | | | | genome |
| AIE11918 | Sierra | May 28, 2014 | Jun. 30, 2014 | Zaire ebolavirus isolate Ebola virus/H.sapiens- |
| | Leone | | | wt/SLE/2014/Makona-G3686.1, complete |
| | | | | genome |
| AIE11927 | Sierra | May 28, 2014 | Jun. 30, 2014 | Zaire ebolavirus isolate Ebola virus/H.sapiens- |
| | Leone | | | wt/SLE/2014/Makona-G3687.1, complete |
| | | | | genome |
| AIG96014 | Sierra | May 31, 2014 | Jul. 25, 2014 | Zaire ebolavirus isolate Ebola virus/H.sapiens- |
| | Leone | | | wt/SLE/2014/Makona-G3707, complete genome |
| AIG96023 | Sierra | Jun. 9, 2014 | Jul. 25, 2014 | Zaire ebolavirus isolate Ebola virus/H.sapiens- |
| | Leone | | | wt/SLE/2014/Makona-G3713.2, complete |
| | | | | genome |
| AIG96032 | Sierra | Jun. 11, 2014 | Jul. 25, 2014 | Zaire ebolavirus isolate Ebola virus/H.sapiens- |
| | Leone | | | wt/SLE/2014/Makona-G3713.3, complete |
| | | | | genome |
| AIG96041 | Sierra | Jun. 13, 2014 | Jul. 25, 2014 | Zaire ebolavirus isolate Ebola virus/H.sapiens- |
| | Leone | | | wt/SLE/2014/Makona-G3713.4, complete |
| | | | | genome |
| AIG96050 | Sierra | Jun. 5, 2014 | Jul. 25, 2014 | Zaire ebolavirus isolate Ebola virus/H.sapiens- |
| | Leone | | | wt/SLE/2014/Makona-G3724, complete genome |
| AIG96059 | Sierra | Jun. 7, 2014 | Jul. 25, 2014 | Zaire ebolavirus isolate Ebola virus/H.sapiens- |
| | Leone | | | wt/SLE/2014/Makona-G3729, complete genome |
| AIG96068 | Sierra | Jun. 7, 2014 | Jul. 25, 2014 | Zaire ebolavirus isolate Ebola virus/H.sapiens- |
| | Leone | | | wt/SLE/2014/Makona-G3734.1, complete |
| | | | | genome |
| AIG96077 | Sierra | Jun. 7, 2014 | Jul. 25, 2014 | Zaire ebolavirus isolate Ebola virus/H.sapiens- |
| | Leone | | | wt/SLE/2014/Makona-G3735.1, complete |
| | | | | genome |
| AIG96086 | Sierra | Jun. 9, 2014 | Jul. 25, 2014 | Zaire ebolavirus isolate Ebola virus/H.sapiens- |
| | Leone | | | wt/SLE/2014/Makona-G3735.2, complete |
| | | | | genome |
| AIG96095 | Sierra | Jun. 10, 2014 | Jul. 25, 2014 | Zaire ebolavirus isolate Ebola virus/H.sapiens- |
| | Leone | | | wt/SLE/2014/Makona-G3750.1, complete |
| | | | | genome |
| AIG96104 | Sierra | Jun. 12, 2014 | Jul. 25, 2014 | Zaire ebolavirus isolate Ebola virus/H.sapiens- |
| | Leone | | | wt/SLE/2014/Makona-G3750.2, complete |
| | | | | genome |
| AIG96113 | Sierra | Jun. 14, 2014 | Jul. 25, 2014 | Zaire ebolavirus isolate Ebola virus/H.sapiens- |
| | Leone | | | wt/SLE/2014/Makona-G3750.3, complete |
| | | | | genome |
| AIG96122 | Sierra | Jun. 10, 2014 | Jul. 25, 2014 | Zaire ebolavirus isolate Ebola virus/H.sapiens- |
| | Leone | | | wt/SLE/2014/Makona-G3752, complete genome |
| AIG96131 | Sierra | Jun. 11, 2014 | Jul. 25, 2014 | Zaire ebolavirus isolate Ebola virus/H.sapiens- |
| | Leone | | | wt/SLE/2014/Makona-G3758, complete genome |
| AIG96140 | Sierra | Jun. 12, 2014 | Jul. 25, 2014 | Zaire ebolavirus isolate Ebola virus/H.sapiens- |
| | Leone | | | wt/SLE/2014/Makona-G3764, complete genome |
| AIG96149 | Sierra | Jun. 14, 2014 | Jul. 25, 2014 | Zaire ebolavirus isolate Ebola virus/H.sapiens- |
| | Leone | | | wt/SLE/2014/Makona-G3765.2, complete |
| | | | | genome |
| AIG96158 | Sierra | Jun. 12, 2014 | Jul. 25, 2014 | Zaire ebolavirus isolate Ebola virus/H.sapiens- |
| | Leone | | | wt/SLE/2014/Makona-G3769.1, complete |
| | | | | genome |
| AIG96167 | Sierra | Jun. 13, 2014 | Jul. 25, 2014 | Zaire ebolavirus isolate Ebola virus/H.sapiens- |
| | Leone | | | wt/SLE/2014/Makona-G3769.2, complete |
| | | | | genome |
| AIG96176 | Sierra | Jun. 15, 2014 | Jul. 25, 2014 | Zaire ebolavirus isolate Ebola virus/H.sapiens- |
| | Leone | | | wt/SLE/2014/Makona-G3769.3, complete |
| | | | | genome |
| AIG96185 | Sierra | Jun. 16, 2014 | Jul. 25, 2014 | Zaire ebolavirus isolate Ebola virus/H.sapiens- |
| | Leone | | | wt/SLE/2014/Makona-G3769.4, complete |
| | | | | genome |
| AIG96194 | Sierra | Jun. 12, 2014 | Jul. 25, 2014 | Zaire ebolavirus isolate Ebola virus/H.sapiens- |
| | Leone | | | wt/SLE/2014/Makona-G3770.1, complete |
| | | | | genome |
| AIG96203 | Sierra | Jun. 14, 2014 | Jul. 25, 2014 | Zaire ebolavirus isolate Ebola virus/H.sapiens- |
| | Leone | | | wt/SLE/2014/Makona-G3770.2, complete |
| | | | | genome |
| AIG96212 | Sierra | Jun. 12, 2014 | Jul. 25, 2014 | Zaire ebolavirus isolate Ebola virus/H.sapiens- |
| | Leone | | | wt/SLE/2014/Makona-G3771, complete genome |
| AIG96221 | Sierra | Jun. 14, 2014 | Jul. 25, 2014 | Zaire ebolavirus isolate Ebola virus/H.sapiens- |
| | Leone | | | wt/SLE/2014/Makona-G3782, complete genome |
| AIG96230 | Sierra | Jun. 14, 2014 | Jul. 25, 2014 | Zaire ebolavirus isolate Ebola virus/H.sapiens- |
| | Leone | | | wt/SLE/2014/Makona-G3786, complete genome |
| AIG96239 | Sierra | Jun. 14, 2014 | Jul. 25, 2014 | Zaire ebolavirus isolate Ebola virus/H.sapiens- |
| | Leone | | | wt/SLE/2014/Makona-G3787, complete genome |
| AIG96248 | Sierra | Jun. 14, 2014 | Jul. 25, 2014 | Zaire ebolavirus isolate Ebola virus/H.sapiens- |
| | Leone | | | wt/SLE/2014/Makona-G3788, complete genome |
| AIG96257 | Sierra | Jun. 14, 2014 | Jul. 25, 2014 | Zaire ebolavirus isolate Ebola virus/H.sapiens- |
| | Leone | | | wt/SLE/2014/Makona-G3789.1, complete |
| | | | | genome |
| AIG96266 | Sierra | Jun. 15, 2014 | Jul. 25, 2014 | Zaire ebolavirus isolate Ebola virus/H.sapiens- |
| | Leone | | | wt/SLE/2014/Makona-G3795, complete genome |
| AIG96275 | Sierra | Jun. 15, 2014 | Jul. 25, 2014 | Zaire ebolavirus isolate Ebola virus/H.sapiens- |
| | Leone | | | wt/SLE/2014/Makona-G3796, complete genome |
| AIG96284 | Sierra | Jun. 15, 2014 | Jul. 25, 2014 | Zaire ebolavirus isolate Ebola virus/H.sapiens- |
| | Leone | | | wt/SLE/2014/Makona-G3798, complete genome |
| AIG96293 | Sierra | Jun. 15, 2014 | Jul. 25, 2014 | Zaire ebolavirus isolate Ebola virus/H.sapiens- |
| | Leone | | | wt/SLE/2014/Makona-G3799, complete genome |
| AIG96302 | Sierra | Jun. 15, 2014 | Jul. 25, 2014 | Zaire ebolavirus isolate Ebola virus/H.sapiens- |
| | Leone | | | wt/SLE/2014/Makona-G3800, complete genome |
| AIG96311 | Sierra | Jun. 15, 2014 | Jul. 25, 2014 | Zaire ebolavirus isolate Ebola virus/H.sapiens- |
| | Leone | | | wt/SLE/2014/Makona-G3805.1, complete |
| | | | | genome |
| AIG96320 | Sierra | Jun. 20, 2014 | Jul. 25, 2014 | Zaire ebolavirus isolate Ebola virus/H.sapiens- |
| | Leone | | | wt/SLE/2014/Makona-G3805.2, complete |
| | | | | genome |
| AIG96329 | Sierra | Jun. 15, 2014 | Jul. 25, 2014 | Zaire ebolavirus isolate Ebola virus/H.sapiens- |
| | Leone | | | wt/SLE/2014/Makona-G3807, complete genome |
| AIG96338 | Sierra | Jun. 15, 2014 | Jul. 25, 2014 | Zaire ebolavirus isolate Ebola virus/H.sapiens- |
| | Leone | | | wt/SLE/2014/Makona-G3808, complete genome |
| AIG96347 | Sierra | Jun. 15, 2014 | Jul. 25, 2014 | Zaire ebolavirus isolate Ebola virus/H.sapiens- |
| | Leone | | | wt/SLE/2014/Makona-G3809, complete genome |
| AIG96356 | Sierra | Jun. 15, 2014 | Jul. 25, 2014 | Zaire ebolavirus isolate Ebola virus/H.sapiens- |
| | Leone | | | wt/SLE/2014/Makona-G3810.1, complete |
| | | | | genome |
| AIG96365 | Sierra | Jun. 17, 2014 | Jul. 25, 2014 | Zaire ebolavirus isolate Ebola virus/H.sapiens- |
| | Leone | | | wt/SLE/2014/Makona-G3810.2, complete |
| | | | | genome |
| AIG96374 | Sierra | Jun. 15, 2014 | Jul. 25, 2014 | Zaire ebolavirus isolate Ebola virus/H.sapiens- |
| | Leone | | | wt/SLE/2014/Makona-G3814, complete genome |
| AIG96383 | Sierra | Jun. 15, 2014 | Jul. 25, 2014 | Zaire ebolavirus isolate Ebola virus/H.sapiens- |
| | Leone | | | wt/SLE/2014/Makona-G3816, complete genome |
| AIG96392 | Sierra | Jun. 15, 2014 | Jul. 25, 2014 | Zaire ebolavirus isolate Ebola virus/H.sapiens- |
| | Leone | | | wt/SLE/2014/Makona-G3817, complete genome |
| AIG96401 | Sierra | Jun. 15, 2014 | Jul. 25, 2014 | Zaire ebolavirus isolate Ebola virus/H.sapiens- |
| | Leone | | | wt/SLE/2014/Makona-G3818, complete genome |
| AIG96410 | Sierra | Jun. 15, 2014 | Jul. 25, 2014 | Zaire ebolavirus isolate Ebola virus/H.sapiens- |
| | Leone | | | wt/SLE/2014/Makona-G3819, complete genome |
| AIG96419 | Sierra | Jun. 15, 2014 | Jul. 25, 2014 | Zaire ebolavirus isolate Ebola virus/H.sapiens- |
| | Leone | | | wt/SLE/2014/Makona-G3820, complete genome |
| AIG96428 | Sierra | Jun. 15, 2014 | Jul. 25, 2014 | Zaire ebolavirus isolate Ebola virus/H.sapiens- |
| | Leone | | | wt/SLE/2014/Makona-G3821, complete genome |
| AIG96437 | Sierra | Jun. 15, 2014 | Jul. 25, 2014 | Zaire ebolavirus isolate Ebola virus/H.sapiens- |
| | Leone | | | wt/SLE/2014/Makona-G3822, complete genome |
| AIG96446 | Sierra | Jun. 15, 2014 | Jul. 25, 2014 | Zaire ebolavirus isolate Ebola virus/H.sapiens- |
| | Leone | | | wt/SLE/2014/Makona-G3823, complete genome |
| AIG96455 | Sierra | Jun. 16, 2014 | Jul. 25, 2014 | Zaire ebolavirus isolate Ebola virus/H.sapiens- |
| | Leone | | | wt/SLE/2014/Makona-G3825.1, complete |
| | | | | genome |
| AIG96464 | Sierra | Jun. 17, 2014 | Jul. 25, 2014 | Zaire ebolavirus isolate Ebola virus/H.sapiens- |
| | Leone | | | wt/SLE/2014/Makona-G3825.2, complete |
| | | | | genome |
| AIG96473 | Sierra | Jun. 16, 2014 | Jul. 25, 2014 | Zaire ebolavirus isolate Ebola virus/H.sapiens- |
| | Leone | | | wt/SLE/2014/Makona-G3826, complete genome |
| AIG96482 | Sierra | Jun. 16, 2014 | Jul. 25, 2014 | Zaire ebolavirus isolate Ebola virus/H.sapiens- |
| | Leone | | | wt/SLE/2014/Makona-G3827, complete genome |
| AIG96491 | Sierra | Jun. 16, 2014 | Jul. 25, 2014 | Zaire ebolavirus isolate Ebola virus/H.sapiens- |
| | Leone | | | wt/SLE/2014/Makona-G3829, complete genome |
| AIG96500 | Sierra | Jun. 16, 2014 | Jul. 25, 2014 | Zaire ebolavirus isolate Ebola virus/H.sapiens- |
| | Leone | | | wt/SLE/2014/Makona-G3831, complete genome |
| AIG96509 | Sierra | Jun. 17, 2014 | Jul. 25, 2014 | Zaire ebolavirus isolate Ebola virus/H.sapiens- |
| | Leone | | | wt/SLE/2014/Makona-G3834, complete genome |
| AIG96518 | Sierra | Jun. 17, 2014 | Jul. 25, 2014 | Zaire ebolavirus isolate Ebola virus/H.sapiens- |
| | Leone | | | wt/SLE/2014/Makona-G3838, complete genome |
| AKC35925 | Sierra | Jun. 16, 2014 | Apr. 19, 2015 | Zaire ebolavirus isolate Ebola virus/H.sapiens- |
| | Leone | | | wt/SLE/2014/Makona-G3838.2, partial genome |
| AIG96527 | Sierra | Jun. 17, 2014 | Jul. 25, 2014 | Zaire ebolavirus isolate Ebola virus/H.sapiens- |
| | Leone | | | wt/SLE/2014/Makona-G3840, complete genome |
| AIG96536 | Sierra | Jun. 17, 2014 | Jul. 25, 2014 | Zaire ebolavirus isolate Ebola virus/H.sapiens- |
| | Leone | | | wt/SLE/2014/Makona-G3841, complete genome |
| AIG96545 | Sierra | Jun. 18, 2014 | Jul. 25, 2014 | Zaire ebolavirus isolate Ebola virus/H.sapiens- |
| | Leone | | | wt/SLE/2014/Makona-G3845, complete genome |
| AKC35934 | Sierra | Jun. 19, 2014 | Apr. 19, 2015 | Zaire ebolavirus isolate Ebola virus/H.sapiens- |
| | Leone | | | wt/SLE/2014/Makona-G3845.2, partial genome |
| AIG96554 | Sierra | Jun. 18, 2014 | Jul. 25, 2014 | Zaire ebolavirus isolate Ebola virus/H.sapiens- |
| | Leone | | | wt/SLE/2014/Makona-G3846, complete genome |
| AIG96563 | Sierra | Jun. 18, 2014 | Jul. 25, 2014 | Zaire ebolavirus isolate Ebola virus/H.sapiens- |
| | Leone | | | wt/SLE/2014/Makona-G3848, complete genome |
| AIG96572 | Sierra | Jun. 18, 2014 | Jul. 25, 2014 | Zaire ebolavirus isolate Ebola virus/H.sapiens- |
| | Leone | | | wt/SLE/2014/Makona-G3850, complete genome |
| AIG96581 | Sierra | Jun. 18, 2014 | Jul. 25, 2014 | Zaire ebolavirus isolate Ebola virus/H.sapiens- |
| | Leone | | | wt/SLE/2014/Makona-G3851, complete genome |
| AKC35943 | Sierra | Jun. 18, 2014 | Apr. 19, 2015 | Zaire ebolavirus isolate Ebola virus/H.sapiens- |
| | Leone | | | wt/SLE/2014/Makona-G3851.2, partial genome |
| AKC35952 | Sierra | Jun. 17, 2014 | Apr. 19, 2015 | Zaire ebolavirus isolate Ebola virus/H.sapiens- |
| | Leone | | | wt/SLE/2014/Makona-G3855.2, partial genome |
| AIG96590 | Sierra | Jun. 18, 2014 | Jul. 25, 2014 | Zaire ebolavirus isolate Ebola virus/H.sapiens- |
| | Leone | | | wt/SLE/2014/Makona-G3856.1, complete |
| | | | | genome |
| AIG96599 | Sierra | Jun. 20, 2014 | Jul. 25, 2014 | Zaire ebolavirus isolate Ebola virus/H.sapiens- |
| | Leone | | | wt/SLE/2014/Makona-G3856.3, complete |
| | | | | genome |
| AIG96608 | Sierra | Jun. 18, 2014 | Jul. 25, 2014 | Zaire ebolavirus isolate Ebola virus/H.sapiens- |
| | Leone | | | wt/SLE/2014/Makona-G3857, complete genome |
| AKA43781 | Sierra | | Apr. 1, 2015 | Zaire ebolavirus isolate Ebola virus/H.sapiens- |
| | Leone | | | wt/SLE/2014/Makona-G3864.1, complete |
| | | | | genome |
| AKC35961 | Sierra | Jun. 19, 2014 | Apr. 19, 2015 | Zaire ebolavirus isolate Ebola virus/H.sapiens- |
| | Leone | | | wt/SLE/2014/Makona-G3886.1, partial genome |
| AKC35970 | Sierra | Jun. 19, 2014 | Apr. 19, 2015 | Zaire ebolavirus isolate Ebola virus/H.sapiens- |
| | Leone | | | wt/SLE/2014/Makona-G3889.1, partial genome |
| AKC35979 | Sierra | Jun. 21, 2014 | Apr. 19, 2015 | Zaire ebolavirus isolate Ebola virus/H.sapiens- |
| | Leone | | | wt/SLE/2014/Makona-G3913.1, partial genome |
| AKC35988 | Sierra | Jun. 21, 2014 | Apr. 19, 2015 | Zaire ebolavirus isolate Ebola virus/H.sapiens- |
| | Leone | | | wt/SLE/2014/Makona-G3917.1, partial genome |
| AKC35997 | Sierra | Jun. 22, 2014 | Apr. 19, 2015 | Zaire ebolavirus isolate Ebola virus/H.sapiens- |
| | Leone | | | wt/SLE/2014/Makona-G3926.2, partial genome |
| AKC36006 | Sierra | Jun. 24, 2014 | Apr. 19, 2015 | Zaire ebolavirus isolate Ebola virus/H.sapiens- |
| | Leone | | | wt/SLE/2014/Makona-G3949.1, partial genome |
| AKC36015 | Sierra | Jun. 24, 2014 | Apr. 19, 2015 | Zaire ebolavirus isolate Ebola virus/H.sapiens- |
| | Leone | | | wt/SLE/2014/Makona-G3950.1, partial genome |
| AKC36024 | Sierra | Jun. 25, 2014 | Apr. 19, 2015 | Zaire ebolavirus isolate Ebola virus/H.sapiens- |
| | Leone | | | wt/SLE/2014/Makona-G3952.1, partial genome |
| AKC36033 | Sierra | Jun. 24, 2014 | Apr. 19, 2015 | Zaire ebolavirus isolate Ebola virus/H.sapiens- |
| | Leone | | | wt/SLE/2014/Makona-G3972.1, partial genome |
| AKC36042 | Sierra | Jul. 5, 2014 | Apr. 19, 2015 | Zaire ebolavirus isolate Ebola virus/H.sapiens- |
| | Leone | | | wt/SLE/2014/Makona-G4132.1, partial genome |
| AKC36051 | Sierra | Jul. 4, 2014 | Apr. 19, 2015 | Zaire ebolavirus isolate Ebola virus/H.sapiens- |
| | Leone | | | wt/SLE/2014/Makona-G4133.1, partial genome |
| AKC36060 | Sierra | Jul. 6, 2014 | Apr. 19, 2015 | Zaire ebolavirus isolate Ebola virus/H.sapiens- |
| | Leone | | | wt/SLE/2014/Makona-G4151.1, partial genome |
| AKC36069 | Sierra | Jul. 7, 2014 | Apr. 19, 2015 | Zaire ebolavirus isolate Ebola virus/H.sapiens- |
| | Leone | | | wt/SLE/2014/Makona-G4190.1, partial genome |
| AKC36078 | Sierra | Jul. 8, 2014 | Apr. 19, 2015 | Zaire ebolavirus isolate Ebola virus/H.sapiens- |
| | Leone | | | wt/SLE/2014/Makona-G4200.1, partial genome |
| AKC36087 | Sierra | Jul. 8, 2014 | Apr. 19, 2015 | Zaire ebolavirus isolate Ebola virus/H.sapiens- |
| | Leone | | | wt/SLE/2014/Makona-G4217.1, partial genome |
| AKC36096 | Sierra | Jul. 9, 2014 | Apr. 19, 2015 | Zaire ebolavirus isolate Ebola virus/H.sapiens- |
| | Leone | | | wt/SLE/2014/Makona-G4221.1, partial genome |
| AKC36105 | Sierra | Jul. 11, 2014 | Apr. 19, 2015 | Zaire ebolavirus isolate Ebola virus/H.sapiens- |
| | Leone | | | wt/SLE/2014/Makona-G4235.1, partial genome |
| AKC36114 | Sierra | Jul. 10, 2014 | Apr. 19, 2015 | Zaire ebolavirus isolate Ebola virus/H.sapiens- |
| | Leone | | | wt/SLE/2014/Makona-G4236.1, partial genome |
| AKC36123 | Sierra | Jul. 11, 2014 | Apr. 19, 2015 | Zaire ebolavirus isolate Ebola virus/H.sapiens- |
| | Leone | | | wt/SLE/2014/Makona-G4250.1, partial genome |
| AKC36132 | Sierra | Jul. 11, 2014 | Apr. 19, 2015 | Zaire ebolavirus isolate Ebola virus/H.sapiens- |
| | Leone | | | wt/SLE/2014/Makona-G4251.1, partial genome |
| AKC36141 | Sierra | Jul. 11, 2014 | Apr. 19, 2015 | Zaire ebolavirus isolate Ebola virus/H.sapiens- |
| | Leone | | | wt/SLE/2014/Makona-G4252.1, partial genome |
| AKC36150 | Sierra | Jul. 11, 2014 | Apr. 19, 2015 | Zaire ebolavirus isolate Ebola virus/H.sapiens- |
| | Leone | | | wt/SLE/2014/Makona-G4254.1, partial genome |
| AKC36159 | Sierra | Jul. 11, 2014 | Apr. 19, 2015 | Zaire ebolavirus isolate Ebola virus/H.sapiens- |
| | Leone | | | wt/SLE/2014/Makona-G4255.1, partial genome |
| AKC36168 | Sierra | Jul. 11, 2014 | Apr. 19, 2015 | Zaire ebolavirus isolate Ebola virus/H.sapiens- |
| | Leone | | | wt/SLE/2014/Makona-G4263.1, partial genome |
| AKC36177 | Sierra | Jul. 11, 2014 | Apr. 19, 2015 | Zaire ebolavirus isolate Ebola virus/H.sapiens- |
| | Leone | | | wt/SLE/2014/Makona-G4264.1, partial genome |
| AKC36186 | Sierra | Jul. 12, 2014 | Apr. 19, 2015 | Zaire ebolavirus isolate Ebola virus/H.sapiens- |
| | Leone | | | wt/SLE/2014/Makona-G4299.1, partial genome |
| AKC36195 | Sierra | Jul. 12, 2014 | Apr. 19, 2015 | Zaire ebolavirus isolate Ebola virus/H.sapiens- |
| | Leone | | | wt/SLE/2014/Makona-G4312.2, partial genome |
| AKC36204 | Sierra | Jul. 13, 2014 | Apr. 19, 2015 | Zaire ebolavirus isolate Ebola virus/H.sapiens- |
| | Leone | | | wt/SLE/2014/Makona-G4316.1, partial genome |
| AKC36213 | Sierra | Jul. 14, 2014 | Apr. 19, 2015 | Zaire ebolavirus isolate Ebola virus/H.sapiens- |
| | Leone | | | wt/SLE/2014/Makona-G4323.2, partial genome |
| AKC36222 | Sierra | Jul. 14, 2014 | Apr. 19, 2015 | Zaire ebolavirus isolate Ebola virus/H.sapiens- |
| | Leone | | | wt/SLE/2014/Makona-G4324.1, partial genome |
| AKC36231 | Sierra | Jul. 14, 2014 | Apr. 19, 2015 | Zaire ebolavirus isolate Ebola virus/H.sapiens- |
| | Leone | | | wt/SLE/2014/Makona-G4325.1, partial genome |
| AKC36240 | Sierra | Jul. 14, 2014 | Apr. 19, 2015 | Zaire ebolavirus isolate Ebola virus/H.sapiens- |
| | Leone | | | wt/SLE/2014/Makona-G4329.1, partial genome |
| AKC36249 | Sierra | Jul. 13, 2014 | Apr. 19, 2015 | Zaire ebolavirus isolate Ebola virus/H.sapiens- |
| | Leone | | | wt/SLE/2014/Makona-G4333.1, partial genome |
| AKC36258 | Sierra | Jul. 14, 2014 | Apr. 19, 2015 | Zaire ebolavirus isolate Ebola virus/H.sapiens- |
| | Leone | | | wt/SLE/2014/Makona-G4334.1, partial genome |
| AKC36267 | Sierra | Jul. 14, 2014 | Apr. 19, 2015 | Zaire ebolavirus isolate Ebola virus/H.sapiens- |
| | Leone | | | wt/SLE/2014/Makona-G4337.1, partial genome |
| AKC36276 | Sierra | Jul. 18, 2014 | Apr. 19, 2015 | Zaire ebolavirus isolate Ebola virus/H.sapiens- |
| | Leone | | | wt/SLE/2014/Makona-G4345.1, partial genome |
| AKC36285 | Sierra | Jul. 15, 2014 | Apr. 19, 2015 | Zaire ebolavirus isolate Ebola virus/H.sapiens- |
| | Leone | | | wt/SLE/2014/Makona-G4347.1, partial genome |
| AKC36294 | Sierra | Jul. 9, 2014 | Apr. 19, 2015 | Zaire ebolavirus isolate Ebola virus/H.sapiens- |
| | Leone | | | wt/SLE/2014/Makona-G4348.1, partial genome |
| AKC36303 | Sierra | Jul. 14, 2014 | Apr. 19, 2015 | Zaire ebolavirus isolate Ebola virus/H.sapiens- |
| | Leone | | | wt/SLE/2014/Makona-G4350.1, partial genome |
| AKC36312 | Sierra | Jul. 15, 2014 | Apr. 19, 2015 | Zaire ebolavirus isolate Ebola virus/H.sapiens- |
| | Leone | | | wt/SLE/2014/Makona-G4380.1, partial genome |
| AKC36321 | Sierra | Jul. 16, 2014 | Apr. 19, 2015 | Zaire ebolavirus isolate Ebola virus/H.sapiens- |
| | Leone | | | wt/SLE/2014/Makona-G4382.1, partial genome |
| AKC36330 | Sierra | Jul. 16, 2014 | Apr. 19, 2015 | Zaire ebolavirus isolate Ebola virus/H.sapiens- |
| | Leone | | | wt/SLE/2014/Makona-G4389.1, partial genome |
| AKC36339 | Sierra | Jul. 18, 2014 | Apr. 19, 2015 | Zaire ebolavirus isolate Ebola virus/H.sapiens- |
| | Leone | | | wt/SLE/2014/Makona-G4406.1, partial genome |
| AKC36348 | Sierra | Jul. 18, 2014 | Apr. 19, 2015 | Zaire ebolavirus isolate Ebola virus/H.sapiens- |
| | Leone | | | wt/SLE/2014/Makona-G4415.1, partial genome |
| AKC36357 | Sierra | Jul. 18, 2014 | Apr. 19, 2015 | Zaire ebolavirus isolate Ebola virus/H.sapiens- |
| | Leone | | | wt/SLE/2014/Makona-G4416.1, partial genome |
| AKC36366 | Sierra | Jul. 19, 2014 | Apr. 19, 2015 | Zaire ebolavirus isolate Ebola virus/H.sapiens- |
| | Leone | | | wt/SLE/2014/Makona-G4419.1, partial genome |
| AKC36375 | Sierra | Jul. 19, 2014 | Apr. 19, 2015 | Zaire ebolavirus isolate Ebola virus/H.sapiens- |
| | Leone | | | wt/SLE/2014/Makona-G4422.1, partial genome |
| AKC36384 | Sierra | Jul. 19, 2014 | Apr. 19, 2015 | Zaire ebolavirus isolate Ebola virus/H.sapiens- |
| | Leone | | | wt/SLE/2014/Makona-G4423.1, partial genome |
| AKC36393 | Sierra | Jul. 19, 2014 | Apr. 19, 2015 | Zaire ebolavirus isolate Ebola virus/H.sapiens- |
| | Leone | | | wt/SLE/2014/Makona-G4424.1, partial genome |
| AKC36402 | Sierra | Jul. 20, 2014 | Apr. 19, 2015 | Zaire ebolavirus isolate Ebola virus/H.sapiens- |
| | Leone | | | wt/SLE/2014/Makona-G4431.1, partial genome |
| AKC36411 | Sierra | Jul. 20, 2014 | Apr. 19, 2015 | Zaire ebolavirus isolate Ebola virus/H.sapiens- |
| | Leone | | | wt/SLE/2014/Makona-G4433.1, partial genome |
| AKC36420 | Sierra | Jul. 20, 2014 | Apr. 19, 2015 | Zaire ebolavirus isolate Ebola virus/H.sapiens- |
| | Leone | | | wt/SLE/2014/Makona-G4437.1, partial genome |
| AKC36429 | Sierra | Jul. 21, 2014 | Apr. 19, 2015 | Zaire ebolavirus isolate Ebola virus/H.sapiens- |
| | Leone | | | wt/SLE/2014/Makona-G4445.1, partial genome |
| AKC36438 | Sierra | Jul. 21, 2014 | Apr. 19, 2015 | Zaire ebolavirus isolate Ebola virus/H.sapiens- |
| | Leone | | | wt/SLE/2014/Makona-G4450.1, partial genome |
| AKC36447 | Sierra | Jul. 21, 2014 | Apr. 19, 2015 | Zaire ebolavirus isolate Ebola virus/H.sapiens- |
| | Leone | | | wt/SLE/2014/Makona-G4454.1, partial genome |
| AKC36456 | Sierra | Aug. 5, 2014 | Apr. 19, 2015 | Zaire ebolavirus isolate Ebola virus/H.sapiens- |
| | Leone | | | wt/SLE/2014/Makona-G4465.1, partial genome |
| AKC36465 | Sierra | Jul. 22, 2014 | Apr. 19, 2015 | Zaire ebolavirus isolate Ebola virus/H.sapiens- |
| | Leone | | | wt/SLE/2014/Makona-G4466.1, partial genome |
| AKC36474 | Sierra | Jul. 26, 2014 | Apr. 19, 2015 | Zaire ebolavirus isolate Ebola virus/H.sapiens- |
| | Leone | | | wt/SLE/2014/Makona-G4527.2, partial genome |
| AKC36483 | Sierra | Aug. 1, 2014 | Apr. 19, 2015 | Zaire ebolavirus isolate Ebola virus/H.sapiens- |
| | Leone | | | wt/SLE/2014/Makona-G4683.1, partial genome |
| AKC36492 | Sierra | Aug. 3, 2014 | Apr. 19, 2015 | Zaire ebolavirus isolate Ebola virus/H.sapiens- |
| | Leone | | | wt/SLE/2014/Makona-G4698.1, partial genome |
| AKC36501 | Sierra | Aug. 3, 2014 | Apr. 19, 2015 | Zaire ebolavirus isolate Ebola virus/H.sapiens- |
| | Leone | | | wt/SLE/2014/Makona-G4701.1, partial genome |
| AKC36510 | Sierra | Aug. 3, 2014 | Apr. 19, 2015 | Zaire ebolavirus isolate Ebola virus/H.sapiens- |
| | Leone | | | wt/SLE/2014/Makona-G4702.1, partial genome |
| AKC36519 | Sierra | Aug. 4, 2014 | Apr. 19, 2015 | Zaire ebolavirus isolate Ebola virus/H.sapiens- |
| | Leone | | | wt/SLE/2014/Makona-G4717.1, partial genome |
| AKC36528 | Sierra | Aug. 4, 2014 | Apr. 19, 2015 | Zaire ebolavirus isolate Ebola virus/H.sapiens- |
| | Leone | | | wt/SLE/2014/Makona-G4725.1, partial genome |
| AKC36537 | Sierra | Aug. 5, 2014 | Apr. 19, 2015 | Zaire ebolavirus isolate Ebola virus/H.sapiens- |
| | Leone | | | wt/SLE/2014/Makona-G4730.1, partial genome |
| AKC36546 | Sierra | Aug. 5, 2014 | Apr. 19, 2015 | Zaire ebolavirus isolate Ebola virus/H.sapiens- |
| | Leone | | | wt/SLE/2014/Makona-G4736.1, partial genome |
| AKC36555 | Sierra | Aug. 5, 2014 | Apr. 19, 2015 | Zaire ebolavirus isolate Ebola virus/H.sapiens- |
| | Leone | | | wt/SLE/2014/Makona-G4748.1, partial genome |
| AKC36564 | Sierra | Aug. 5, 2014 | Apr. 19, 2015 | Zaire ebolavirus isolate Ebola virus/H.sapiens- |
| | Leone | | | wt/SLE/2014/Makona-G4751.1, partial genome |
| AKC36572 | Sierra | Aug. 9, 2014 | Apr. 19, 2015 | Zaire ebolavirus isolate Ebola virus/H.sapiens- |
| | Leone | | | wt/SLE/2014/Makona-G4837.1, partial genome |
| AKC36581 | Sierra | Aug. 10, 2014 | Apr. 19, 2015 | Zaire ebolavirus isolate Ebola virus/H.sapiens- |
| | Leone | | | wt/SLE/2014/Makona-G4856.1, partial genome |
| AKC36590 | Sierra | Aug. 10, 2014 | Apr. 19, 2015 | Zaire ebolavirus isolate Ebola virus/H.sapiens- |
| | Leone | | | wt/SLE/2014/Makona-G4861.1, partial genome |
| AKC36599 | Sierra | Aug. 10, 2014 | Apr. 19, 2015 | Zaire ebolavirus isolate Ebola virus/H.sapiens- |
| | Leone | | | wt/SLE/2014/Makona-G4868.1, partial genome |
| AKC36608 | Sierra | Aug. 11, 2014 | Apr. 19, 2015 | Zaire ebolavirus isolate Ebola virus/H.sapiens- |
| | Leone | | | wt/SLE/2014/Makona-G4886.1, partial genome |
| AKC36617 | Sierra | Aug. 12, 2014 | Apr. 19, 2015 | Zaire ebolavirus isolate Ebola virus/H.sapiens- |
| | Leone | | | wt/SLE/2014/Makona-G4907.1, partial genome |
| AKC36626 | Sierra | Aug. 13, 2014 | Apr. 19, 2015 | Zaire ebolavirus isolate Ebola virus/H.sapiens- |
| | Leone | | | wt/SLE/2014/Makona-G4937.1, partial genome |
| AKC36635 | Sierra | Aug. 12, 2014 | Apr. 19, 2015 | Zaire ebolavirus isolate Ebola virus/H.sapiens- |
| | Leone | | | wt/SLE/2014/Makona-G4942.1, partial genome |
| AKC36644 | Sierra | Aug. 13, 2014 | Apr. 19, 2015 | Zaire ebolavirus isolate Ebola virus/H.sapiens- |
| | Leone | | | wt/SLE/2014/Makona-G4946.1, partial genome |
| AKC36653 | Sierra | Aug. 13, 2014 | Apr. 19, 2015 | Zaire ebolavirus isolate Ebola virus/H.sapiens- |
| | Leone | | | wt/SLE/2014/Makona-G4955.1, partial genome |
| AKC36662 | Sierra | Aug. 13, 2014 | Apr. 19, 2015 | Zaire ebolavirus isolate Ebola virus/H.sapiens- |
| | Leone | | | wt/SLE/2014/Makona-G4956.1, partial genome |
| AKC36671 | Sierra | Aug. 14, 2014 | Apr. 19, 2015 | Zaire ebolavirus isolate Ebola virus/H.sapiens- |
| | Leone | | | wt/SLE/2014/Makona-G4960.1, partial genome |
| AKC36680 | Sierra | Aug. 14, 2014 | Apr. 19, 2015 | Zaire ebolavirus isolate Ebola virus/H.sapiens- |
| | Leone | | | wt/SLE/2014/Makona-G4971.1, partial genome |
| AKC36689 | Sierra | Aug. 14, 2014 | Apr. 19, 2015 | Zaire ebolavirus isolate Ebola virus/H.sapiens- |
| | Leone | | | wt/SLE/2014/Makona-G4972.1, partial genome |
| AKC36698 | Sierra | Aug. 12, 2014 | Apr. 19, 2015 | Zaire ebolavirus isolate Ebola virus/H.sapiens- |
| | Leone | | | wt/SLE/2014/Makona-G4973.1, partial genome |
| AKC36707 | Sierra | Aug. 14, 2014 | Apr. 19, 2015 | Zaire ebolavirus isolate Ebola virus/H.sapiens- |
| | Leone | | | wt/SLE/2014/Makona-G4981.1, partial genome |
| AKC36716 | Sierra | Aug. 14, 2014 | Apr. 19, 2015 | Zaire ebolavirus isolate Ebola virus/H.sapiens- |
| | Leone | | | wt/SLE/2014/Makona-G4982.1, partial genome |
| AKC36725 | Sierra | Aug. 15, 2014 | Apr. 19, 2015 | Zaire ebolavirus isolate Ebola virus/H.sapiens- |
| | Leone | | | wt/SLE/2014/Makona-G4994.1, partial genome |
| AKC36734 | Sierra | Aug. 15, 2014 | Apr. 19, 2015 | Zaire ebolavirus isolate Ebola virus/H.sapiens- |
| | Leone | | | wt/SLE/2014/Makona-G4996.1, partial genome |
| AKC36743 | Sierra | Aug. 15, 2014 | Apr. 19, 2015 | Zaire ebolavirus isolate Ebola virus/H.sapiens- |
| | Leone | | | wt/SLE/2014/Makona-G4999.1, partial genome |
| AKC36752 | Sierra | Aug. 15, 2014 | Apr. 19, 2015 | Zaire ebolavirus isolate Ebola virus/H.sapiens- |
| | Leone | | | wt/SLE/2014/Makona-G5012.3, partial genome |
| AKC36761 | Sierra | Aug. 16, 2014 | Apr. 19, 2015 | Zaire ebolavirus isolate Ebola virus/H.sapiens- |
| | Leone | | | wt/SLE/2014/Makona-G5016.1, partial genome |
| AKC36770 | Sierra | Aug. 16, 2014 | Apr. 19, 2015 | Zaire ebolavirus isolate Ebola virus/H.sapiens- |
| | Leone | | | wt/SLE/2014/Makona-G5019.1, partial genome |
| AKC36779 | Sierra | Aug. 17, 2014 | Apr. 19, 2015 | Zaire ebolavirus isolate Ebola virus/H.sapiens- |
| | Leone | | | wt/SLE/2014/Makona-G5039.1, partial genome |
| AKC36788 | Sierra | Aug. 18, 2014 | Apr. 19, 2015 | Zaire ebolavirus isolate Ebola virus/H.sapiens- |
| | Leone | | | wt/SLE/2014/Makona-G5059.1, partial genome |
| AKC36797 | Sierra | Aug. 17, 2014 | Apr. 19, 2015 | Zaire ebolavirus isolate Ebola virus/H.sapiens- |
| | Leone | | | wt/SLE/2014/Makona-G5064.1, partial genome |
| AKC36806 | Sierra | Aug. 19, 2014 | Apr. 19, 2015 | Zaire ebolavirus isolate Ebola virus/H.sapiens- |
| | Leone | | | wt/SLE/2014/Makona-G5112.1, partial genome |
| AKC36815 | Sierra | Aug. 18, 2014 | Apr. 19, 2015 | Zaire ebolavirus isolate Ebola virus/H.sapiens- |
| | Leone | | | wt/SLE/2014/Makona-G5114.1, partial genome |
| AKC36824 | Sierra | Aug. 24, 2014 | Apr. 19, 2015 | Zaire ebolavirus isolate Ebola virus/H.sapiens- |
| | Leone | | | wt/SLE/2014/Makona-G5119.1, partial genome |
| AKC36833 | Sierra | Aug. 22, 2014 | Apr. 19, 2015 | Zaire ebolavirus isolate Ebola virus/H.sapiens- |
| | Leone | | | wt/SLE/2014/Makona-G5244.1, partial genome |
| AKC36842 | Sierra | Aug. 25, 2014 | Apr. 19, 2015 | Zaire ebolavirus isolate Ebola virus/H.sapiens- |
| | Leone | | | wt/SLE/2014/Makona-G5295.1, partial genome |
| AKC36851 | Sierra | Aug. 25, 2014 | Apr. 19, 2015 | Zaire ebolavirus isolate Ebola virus/H.sapiens- |
| | Leone | | | wt/SLE/2014/Makona-G5296.1, partial genome |
| AKC36860 | Sierra | Aug. 28, 2014 | Apr. 19, 2015 | Zaire ebolavirus isolate Ebola virus/H.sapiens- |
| | Leone | | | wt/SLE/2014/Makona-G5304.1, partial genome |
| AKC36869 | Sierra | Aug. 28, 2014 | Apr. 19, 2015 | Zaire ebolavirus isolate Ebola virus/H.sapiens- |
| | Leone | | | wt/SLE/2014/Makona-G5364.1, partial genome |
| AKC36878 | Sierra | Aug. 28, 2014 | Apr. 19, 2015 | Zaire ebolavirus isolate Ebola virus/H.sapiens- |
| | Leone | | | wt/SLE/2014/Makona-G5370.1, partial genome |
| AKC36887 | Sierra | Sep. 4, 2014 | Apr. 19, 2015 | Zaire ebolavirus isolate Ebola virus/H.sapiens- |
| | Leone | | | wt/SLE/2014/Makona-G5516.1, partial genome |
| AKC36896 | Sierra | Sep. 5, 2014 | Apr. 19, 2015 | Zaire ebolavirus isolate Ebola virus/H.sapiens- |
| | Leone | | | wt/SLE/2014/Makona-G5520.1, partial genome |
| AKC36905 | Sierra | Sep. 5, 2014 | Apr. 19, 2015 | Zaire ebolavirus isolate Ebola virus/H.sapiens- |
| | Leone | | | wt/SLE/2014/Makona-G5529.1, partial genome |
| AKC36914 | Sierra | Sep. 8, 2014 | Apr. 19, 2015 | Zaire ebolavirus isolate Ebola virus/H.sapiens- |
| | Leone | | | wt/SLE/2014/Makona-G5570.1, partial genome |
| AKC36923 | Sierra | Sep. 8, 2014 | Apr. 19, 2015 | Zaire ebolavirus isolate Ebola virus/H.sapiens- |
| | Leone | | | wt/SLE/2014/Makona-G5571.1, partial genome |
| AKC36932 | Sierra | Sep. 10, 2014 | Apr. 19, 2015 | Zaire ebolavirus isolate Ebola virus/H.sapiens- |
| | Leone | | | wt/SLE/2014/Makona-G5617.1, partial genome |
| AKC36941 | Sierra | Sep. 11, 2014 | Apr. 19, 2015 | Zaire ebolavirus isolate Ebola virus/H.sapiens- |
| | Leone | | | wt/SLE/2014/Makona-G5640.1, partial genome |
| AKC36950 | Sierra | Sep. 11, 2014 | Apr. 19, 2015 | Zaire ebolavirus isolate Ebola virus/H.sapiens- |
| | Leone | | | wt/SLE/2014/Makona-G5644.1, partial genome |
| AKC36959 | Sierra | Sep. 11, 2014 | Apr. 19, 2015 | Zaire ebolavirus isolate Ebola virus/H.sapiens- |
| | Leone | | | wt/SLE/2014/Makona-G5647.1, partial genome |
| AKC36968 | Sierra | Sep. 13, 2014 | Apr. 19, 2015 | Zaire ebolavirus isolate Ebola virus/H.sapiens- |
| | Leone | | | wt/SLE/2014/Makona-G5684.1, partial genome |
| AKC36977 | Sierra | Sep. 13, 2014 | Apr. 19, 2015 | Zaire ebolavirus isolate Ebola virus/H.sapiens- |
| | Leone | | | wt/SLE/2014/Makona-G5685.1, partial genome |
| AKC36986 | Sierra | Sep. 14, 2014 | Apr. 19, 2015 | Zaire ebolavirus isolate Ebola virus/H.sapiens- |
| | Leone | | | wt/SLE/2014/Makona-G5691.1, partial genome |
| AKC36995 | Sierra | Sep. 15, 2014 | Apr. 19, 2015 | Zaire ebolavirus isolate Ebola virus/H.sapiens- |
| | Leone | | | wt/SLE/2014/Makona-G5723.1, partial genome |
| AKC37004 | Sierra | Sep. 16, 2014 | Apr. 19, 2015 | Zaire ebolavirus isolate Ebola virus/H.sapiens- |
| | Leone | | | wt/SLE/2014/Makona-G5731.1, partial genome |
| AKC37013 | Sierra | Sep. 16, 2014 | Apr. 19, 2015 | Zaire ebolavirus isolate Ebola virus/H.sapiens- |
| | Leone | | | wt/SLE/2014/Makona-G5737.1, partial genome |
| AKC37022 | Sierra | Sep. 15, 2014 | Apr. 19, 2015 | Zaire ebolavirus isolate Ebola virus/H.sapiens- |
| | Leone | | | wt/SLE/2014/Makona-G5738.1, partial genome |
| AKC37031 | Sierra | Sep. 17, 2014 | Apr. 19, 2015 | Zaire ebolavirus isolate Ebola virus/H.sapiens- |
| | Leone | | | wt/SLE/2014/Makona-G5743.1, partial genome |
| AKC37040 | Sierra | Sep. 18, 2014 | Apr. 19, 2015 | Zaire ebolavirus isolate Ebola virus/H.sapiens- |
| | Leone | | | wt/SLE/2014/Makona-G5756.1, partial genome |
| AKC37049 | Sierra | Sep. 16, 2014 | Apr. 19, 2015 | Zaire ebolavirus isolate Ebola virus/H.sapiens- |
| | Leone | | | wt/SLE/2014/Makona-G5763.1, partial genome |
| AKC37058 | Sierra | Sep. 16, 2014 | Apr. 19, 2015 | Zaire ebolavirus isolate Ebola virus/H.sapiens- |
| | Leone | | | wt/SLE/2014/Makona-G5765.1, partial genome |
| AKC37067 | Sierra | Sep. 16, 2014 | Apr. 19, 2015 | Zaire ebolavirus isolate Ebola virus/H.sapiens- |
| | Leone | | | wt/SLE/2014/Makona-G5767.1, partial genome |
| AKC37076 | Sierra | Sep. 21, 2014 | Apr. 19, 2015 | Zaire ebolavirus isolate Ebola virus/H.sapiens- |
| | Leone | | | wt/SLE/2014/Makona-G5844.1, partial genome |
| AKC37085 | Sierra | Sep. 21, 2014 | Apr. 19, 2015 | Zaire ebolavirus isolate Ebola virus/H.sapiens- |
| | Leone | | | wt/SLE/2014/Makona-G5853.1, partial genome |
| AKC37094 | Sierra | Sep. 22, 2014 | Apr. 19, 2015 | Zaire ebolavirus isolate Ebola virus/H.sapiens- |
| | Leone | | | wt/SLE/2014/Makona-G5879.1, partial genome |
| AKC37103 | Sierra | Sep. 22, 2014 | Apr. 19, 2015 | Zaire ebolavirus isolate Ebola virus/H.sapiens- |
| | Leone | | | wt/SLE/2014/Makona-G5898.1, partial genome |
| AKC37112 | Sierra | Sep. 25, 2014 | Apr. 19, 2015 | Zaire ebolavirus isolate Ebola virus/H.sapiens- |
| | Leone | | | wt/SLE/2014/Makona-G5982.1, partial genome |
| AKC37121 | Sierra | Sep. 25, 2014 | Apr. 19, 2015 | Zaire ebolavirus isolate Ebola virus/H.sapiens- |
| | Leone | | | wt/SLE/2014/Makona-G5983.1, partial genome |
| AKC37139 | Sierra | Sep. 25, 2014 | Apr. 19, 2015 | Zaire ebolavirus isolate Ebola virus/H.sapiens- |
| | Leone | | | wt/SLE/2014/Makona-G5986.1, partial genome |
| AKC37148 | Sierra | Sep. 25, 2014 | Apr. 19, 2015 | Zaire ebolavirus isolate Ebola virus/H.sapiens- |
| | Leone | | | wt/SLE/2014/Makona-G5988.1, partial genome |
| AKC37157 | Sierra | Sep. 25, 2014 | Apr. 19, 2015 | Zaire ebolavirus isolate Ebola virus/H.sapiens- |
| | Leone | | | wt/SLE/2014/Makona-G5996.1, partial genome |
| AKC37166 | Sierra | Sep. 25, 2014 | Apr. 19, 2015 | Zaire ebolavirus isolate Ebola virus/H.sapiens- |
| | Leone | | | wt/SLE/2014/Makona-G5997.1, partial genome |
| AKC37175 | Sierra | Sep. 25, 2014 | Apr. 19, 2015 | Zaire ebolavirus isolate Ebola virus/H.sapiens- |
| | Leone | | | wt/SLE/2014/Makona-G5998.1, partial genome |
| AKC37184 | Sierra | Sep. 25, 2014 | Apr. 19, 2015 | Zaire ebolavirus isolate Ebola virus/H.sapiens- |
| | Leone | | | wt/SLE/2014/Makona-G6012.1, partial genome |
| AKC37193 | Sierra | Sep. 25, 2014 | Apr. 19, 2015 | Zaire ebolavirus isolate Ebola virus/H.sapiens- |
| | Leone | | | wt/SLE/2014/Makona-G6020.1, partial genome |
| AKC37202 | Sierra | Sep. 25, 2014 | Apr. 19, 2015 | Zaire ebolavirus isolate Ebola virus/H.sapiens- |
| | Leone | | | wt/SLE/2014/Makona-G6060.1, partial genome |
| AKC37211 | Sierra | Sep. 25, 2014 | Apr. 19, 2015 | Zaire ebolavirus isolate Ebola virus/H.sapiens- |
| | Leone | | | wt/SLE/2014/Makona-G6062.1, partial genome |
| AKC37220 | Sierra | Sep. 25, 2014 | Apr. 19, 2015 | Zaire ebolavirus isolate Ebola virus/H.sapiens- |
| | Leone | | | wt/SLE/2014/Makona-G6069.1, partial genome |
| AKC37229 | Sierra | Sep. 27, 2014 | Apr. 19, 2015 | Zaire ebolavirus isolate Ebola virus/H.sapiens- |
| | Leone | | | wt/SLE/2014/Makona-G6089.1, partial genome |
| AKC37238 | Sierra | Sep. 27, 2014 | Apr. 19, 2015 | Zaire ebolavirus isolate Ebola virus/H.sapiens- |
| | Leone | | | wt/SLE/2014/Makona-G6091.1, partial genome |
| AKC37247 | Sierra | Sep. 27, 2014 | Apr. 19, 2015 | Zaire ebolavirus isolate Ebola virus/H.sapiens- |
| | Leone | | | wt/SLE/2014/Makona-G6095.1, partial genome |
| AKC37256 | Sierra | Sep. 28, 2014 | Apr. 19, 2015 | Zaire ebolavirus isolate Ebola virus/H.sapiens- |
| | Leone | | | wt/SLE/2014/Makona-G6103.1, partial genome |
| AKC37265 | Sierra | Sep. 28, 2014 | Apr. 19, 2015 | Zaire ebolavirus isolate Ebola virus/H.sapiens- |
| | Leone | | | wt/SLE/2014/Makona-G6104.1, partial genome |
| AIG96617 | Sierra | Jun. 4, 2014 | Jul. 25, 2014 | Zaire ebolavirus isolate Ebola virus/H.sapiens- |
| | Leone | | | wt/SLE/2014/Makona-NM042.1, complete |
| | | | | genome |
| AIG96626 | Sierra | Jun. 9, 2014 | Jul. 25, 2014 | Zaire ebolavirus isolate Ebola virus/H.sapiens- |
| | Leone | | | wt/SLE/2014/Makona-NM042.2, complete |
| | | | | genome |
| AIG96635 | Sierra | Jun. 12, 2014 | Jul. 25, 2014 | Zaire ebolavirus isolate Ebola virus/H.sapiens- |
| | Leone | | | wt/SLE/2014/Makona-NM042.3, complete |
| | | | | genome |
| AJP14319 | Sierra | Sep. 27, 2014 | Mar. 1, 2015 | Zaire ebolavirus isolate Ebolavirus/H.sapiens- |
| | Leone | | | wt/SLE/2014/Makona-J0001, partial genome |
| AJP14355 | Sierra | Sep. 27, 2014 | Mar. 1, 2015 | Zaire ebolavirus isolate Ebolavirus/H.sapiens- |
| | Leone | | | wt/SLE/2014/Makona-J0002, partial genome |
| AJP14453 | Sierra | Sep. 28, 2014 | Mar. 1, 2015 | Zaire ebolavirus isolate Ebolavirus/H.sapiens- |
| | Leone | | | wt/SLE/2014/Makona-J0003, partial genome |
| AJP14606 | Sierra | Sep. 28, 2014 | Mar. 1, 2015 | Zaire ebolavirus isolate Ebolavirus/H.sapiens- |
| | Leone | | | wt/SLE/2014/Makona-J0004, partial genome |
| AJP14247 | Sierra | Sep. 26, 2014 | Mar. 1, 2015 | Zaire ebolavirus isolate Ebolavirus/H.sapiens- |
| | Leone | | | wt/SLE/2014/Makona-J0005, partial genome |
| AJP14265 | Sierra | Sep. 28, 2014 | Mar. 1, 2015 | Zaire ebolavirus isolate Ebolavirus/H.sapiens- |
| | Leone | | | wt/SLE/2014/Makona-J0006, partial genome |
| AJP14274 | Sierra | Sep. 27, 2014 | Mar. 1, 2015 | Zaire ebolavirus isolate Ebolavirus/H.sapiens- |
| | Leone | | | wt/SLE/2014/Makona-J0007, partial genome |
| AJP15056 | Sierra | Sep. 29, 2014 | Mar. 1, 2015 | Zaire ebolavirus isolate Ebolavirus/H.sapiens- |
| | Leone | | | wt/SLE/2014/Makona-J0008, partial genome |
| AJP15200 | Sierra | Sep. 28, 2014 | Mar. 1, 2015 | Zaire ebolavirus isolate Ebolavirus/H.sapiens- |
| | Leone | | | wt/SLE/2014/Makona-J0009, partial genome |
| AJP14346 | Sierra | Sep. 28, 2014 | Mar. 1, 2015 | Zaire ebolavirus isolate Ebolavirus/H.sapiens- |
| | Leone | | | wt/SLE/2014/Makona-J0010, partial genome |
| AJP14364 | Sierra | Sep. 29, 2014 | Mar. 1, 2015 | Zaire ebolavirus isolate Ebolavirus/H.sapiens- |
| | Leone | | | wt/SLE/2014/Makona-J0011, partial genome |
| AJP14373 | Sierra | Sep. 29, 2014 | Mar. 1, 2015 | Zaire ebolavirus isolate Ebolavirus/H.sapiens- |
| | Leone | | | wt/SLE/2014/Makona-J0012, partial genome |
| AJP14382 | Sierra | Sep. 28, 2014 | Mar. 1, 2015 | Zaire ebolavirus isolate Ebolavirus/H.sapiens- |
| | Leone | | | wt/SLE/2014/Makona-J0013, partial genome |
| AJP15254 | Sierra | Sep. 29, 2014 | Mar. 1, 2015 | Zaire ebolavirus isolate Ebolavirus/H.sapiens- |
| | Leone | | | wt/SLE/2014/Makona-J0014, partial genome |
| AJP15263 | Sierra | Sep. 26, 2014 | Mar. 1, 2015 | Zaire ebolavirus isolate Ebolavirus/H.sapiens- |
| | Leone | | | wt/SLE/2014/Makona-J0015, partial genome |
| AJP15272 | Sierra | Sep. 30, 2014 | Mar. 1, 2015 | Zaire ebolavirus isolate Ebolavirus/H.sapiens- |
| | Leone | | | wt/SLE/2014/Makona-J0016, partial genome |
| AJP15317 | Sierra | Sep. 26, 2014 | Mar. 1, 2015 | Zaire ebolavirus isolate Ebolavirus/H.sapiens- |
| | Leone | | | wt/SLE/2014/Makona-J0017, partial genome |
| AJP14391 | Sierra | Sep. 30, 2014 | Mar. 1, 2015 | Zaire ebolavirus isolate Ebolavirus/H.sapiens- |
| | Leone | | | wt/SLE/2014/Makona-J0018, partial genome |
| AJP15380 | Sierra | Sep. 25, 2014 | Mar. 1, 2015 | Zaire ebolavirus isolate Ebolavirus/H.sapiens- |
| | Leone | | | wt/SLE/2014/Makona-J0019, partial genome |
| AJP15389 | Sierra | Sep. 25, 2014 | Mar. 1, 2015 | Zaire ebolavirus isolate Ebolavirus/H.sapiens- |
| | Leone | | | wt/SLE/2014/Makona-J0020, partial genome |
| AJP15398 | Sierra | Sep. 29, 2014 | Mar. 1, 2015 | Zaire ebolavirus isolate Ebolavirus/H.sapiens- |
| | Leone | | | wt/SLE/2014/Makona-J0021, partial genome |
| AJP15407 | Sierra | Sep. 25, 2014 | Mar. 1, 2015 | Zaire ebolavirus isolate Ebolavirus/H.sapiens- |
| | Leone | | | wt/SLE/2014/Makona-J0022, partial genome |
| AJP14462 | Sierra | Oct. 1, 2014 | Mar. 1, 2015 | Zaire ebolavirus isolate Ebolavirus/H.sapiens- |
| | Leone | | | wt/SLE/2014/Makona-J0023, partial genome |
| AJP14561 | Sierra | Sep. 29, 2014 | Mar. 1, 2015 | Zaire ebolavirus isolate Ebolavirus/H.sapiens- |
| | Leone | | | wt/SLE/2014/Makona-J0024, partial genome |
| AJP14588 | Sierra | Oct. 2, 2014 | Mar. 1, 2015 | Zaire ebolavirus isolate Ebolavirus/H.sapiens- |
| | Leone | | | wt/SLE/2014/Makona-J0025, partial genome |
| AJP14624 | Sierra | Oct. 2, 2014 | Mar. 1, 2015 | Zaire ebolavirus isolate Ebolavirus/H.sapiens- |
| | Leone | | | wt/SLE/2014/Makona-J0026, partial genome |
| AJP14696 | Sierra | Oct. 2, 2014 | Mar. 1, 2015 | Zaire ebolavirus isolate Ebolavirus/H.sapiens- |
| | Leone | | | wt/SLE/2014/Makona-J0027, partial genome |
| AJP14741 | Sierra | Oct. 2, 2014 | Mar. 1, 2015 | Zaire ebolavirus isolate Ebolavirus/H.sapiens- |
| | Leone | | | wt/SLE/2014/Makona-J0028, partial genome |
| AJP14786 | Sierra | Sep. 29, 2014 | Mar. 1, 2015 | Zaire ebolavirus isolate Ebolavirus/H.sapiens- |
| | Leone | | | wt/SLE/2014/Makona-J0029, partial genome |
| AJP14049 | Sierra | Oct. 3, 2014 | Mar. 1, 2015 | Zaire ebolavirus isolate Ebolavirus/H.sapiens- |
| | Leone | | | wt/SLE/2014/Makona-J0030, partial genome |
| AJP14058 | Sierra | Oct. 3, 2014 | Mar. 1, 2015 | Zaire ebolavirus isolate Ebolavirus/H.sapiens- |
| | Leone | | | wt/SLE/2014/Makona-J0031, partial genome |
| AJP14813 | Sierra | Sep. 30, 2014 | Mar. 1, 2015 | Zaire ebolavirus isolate Ebolavirus/H.sapiens- |
| | Leone | | | wt/SLE/2014/Makona-J0032, partial genome |
| AJP14822 | Sierra | Oct. 3, 2014 | Mar. 1, 2015 | Zaire ebolavirus isolate Ebolavirus/H.sapiens- |
| | Leone | | | wt/SLE/2014/Makona-J0033, partial genome |
| AJP14067 | Sierra | Sep. 30, 2014 | Mar. 1, 2015 | Zaire ebolavirus isolate Ebolavirus/H.sapiens- |
| | Leone | | | wt/SLE/2014/Makona-J0034, partial genome |
| AJP14076 | Sierra | Oct. 1, 2014 | Mar. 1, 2015 | Zaire ebolavirus isolate Ebolavirus/H.sapiens- |
| | Leone | | | wt/SLE/2014/Makona-J0035, partial genome |
| AJP14840 | Sierra | Oct. 3, 2014 | Mar. 1, 2015 | Zaire ebolavirus isolate Ebolavirus/H.sapiens- |
| | Leone | | | wt/SLE/2014/Makona-J0036, partial genome |
| AJP14130 | Sierra | Oct. 3, 2014 | Mar. 1, 2015 | Zaire ebolavirus isolate Ebolavirus/H.sapiens- |
| | Leone | | | wt/SLE/2014/Makona-J0037, partial genome |
| AJP14157 | Sierra | Sep. 30, 2014 | Mar. 1, 2015 | Zaire ebolavirus isolate Ebolavirus/H.sapiens- |
| | Leone | | | wt/SLE/2014/Makona-J0038, partial genome |
| AJP14175 | Sierra | Oct. 3, 2014 | Mar. 1, 2015 | Zaire ebolavirus isolate Ebolavirus/H.sapiens- |
| | Leone | | | wt/SLE/2014/Makona-J0039, partial genome |
| AJP14256 | Sierra | Oct. 4, 2014 | Mar. 1, 2015 | Zaire ebolavirus isolate Ebolavirus/H.sapiens- |
| | Leone | | | wt/SLE/2014/Makona-J0040, partial genome |
| AJP14984 | Sierra | Oct. 4, 2014 | Mar. 1, 2015 | Zaire ebolavirus isolate Ebolavirus/H.sapiens- |
| | Leone | | | wt/SLE/2014/Makona-J0041, partial genome |
| AJP14993 | Sierra | Oct. 4, 2014 | Mar. 1, 2015 | Zaire ebolavirus isolate Ebolavirus/H.sapiens- |
| | Leone | | | wt/SLE/2014/Makona-J0042, partial genome |
| AJP15002 | Sierra | Oct. 4, 2014 | Mar. 1, 2015 | Zaire ebolavirus isolate Ebolavirus/H.sapiens- |
| | Leone | | | wt/SLE/2014/Makona-J0043, partial genome |
| AJP15011 | Sierra | Oct. 5, 2014 | Mar. 1, 2015 | Zaire ebolavirus isolate Ebolavirus/H.sapiens- |
| | Leone | | | wt/SLE/2014/Makona-J0044, partial genome |
| AJP15020 | Sierra | Oct. 5, 2014 | Mar. 1, 2015 | Zaire ebolavirus isolate Ebolavirus/H.sapiens- |
| | Leone | | | wt/SLE/2014/Makona-J0045, partial genome |
| AJP15029 | Sierra | Oct. 7, 2014 | Mar. 1, 2015 | Zaire ebolavirus isolate Ebolavirus/H.sapiens- |
| | Leone | | | wt/SLE/2014/Makona-J0046, partial genome |
| AJP14283 | Sierra | Oct. 5, 2014 | Mar. 1, 2015 | Zaire ebolavirus isolate Ebolavirus/H.sapiens- |
| | Leone | | | wt/SLE/2014/Makona-J0047, partial genome |
| AJP15038 | Sierra | Oct. 5, 2014 | Mar. 1, 2015 | Zaire ebolavirus isolate Ebolavirus/H.sapiens- |
| | Leone | | | wt/SLE/2014/Makona-J0048, partial genome |
| AJP15047 | Sierra | Oct. 7, 2014 | Mar. 1, 2015 | Zaire ebolavirus isolate Ebolavirus/H.sapiens- |
| | Leone | | | wt/SLE/2014/Makona-J0049, partial genome |
| AJP15065 | Sierra | Oct. 5, 2014 | Mar. 1, 2015 | Zaire ebolavirus isolate Ebolavirus/H.sapiens- |
| | Leone | | | wt/SLE/2014/Makona-J0050, partial genome |
| AJP14292 | Sierra | Oct. 7, 2014 | Mar. 1, 2015 | Zaire ebolavirus isolate Ebolavirus/H.sapiens- |
| | Leone | | | wt/SLE/2014/Makona-J0051, partial genome |
| AJP15074 | Sierra | Oct. 8, 2014 | Mar. 1, 2015 | Zaire ebolavirus isolate Ebolavirus/H.sapiens- |
| | Leone | | | wt/SLE/2014/Makona-J0052, partial genome |
| AJP15083 | Sierra | Oct. 6, 2014 | Mar. 1, 2015 | Zaire ebolavirus isolate Ebolavirus/H.sapiens- |
| | Leone | | | wt/SLE/2014/Makona-J0053, partial genome |
| AJP15092 | Sierra | Oct. 6, 2014 | Mar. 1, 2015 | Zaire ebolavirus isolate Ebolavirus/H.sapiens- |
| | Leone | | | wt/SLE/2014/Makona-J0054, partial genome |
| AJP15101 | Sierra | Oct. 6, 2014 | Mar. 1, 2015 | Zaire ebolavirus isolate Ebolavirus/H.sapiens- |
| | Leone | | | wt/SLE/2014/Makona-J0055, partial genome |
| AJP14301 | Sierra | Oct. 8, 2014 | Mar. 1, 2015 | Zaire ebolavirus isolate Ebolavirus/H.sapiens- |
| | Leone | | | wt/SLE/2014/Makona-J0056, partial genome |
| AJP14310 | Sierra | Oct. 5, 2014 | Mar. 1, 2015 | Zaire ebolavirus isolate Ebolavirus/H.sapiens- |
| | Leone | | | wt/SLE/2014/Makona-J0057, partial genome |
| AJP15110 | Sierra | Oct. 8, 2014 | Mar. 1, 2015 | Zaire ebolavirus isolate Ebolavirus/H.sapiens- |
| | Leone | | | wt/SLE/2014/Makona-J0058, partial genome |
| AJP15119 | Sierra | Oct. 8, 2014 | Mar. 1, 2015 | Zaire ebolavirus isolate Ebolavirus/H.sapiens- |
| | Leone | | | wt/SLE/2014/Makona-J0059, partial genome |
| AJP15128 | Sierra | Oct. 6, 2014 | Mar. 1, 2015 | Zaire ebolavirus isolate Ebolavirus/H.sapiens- |
| | Leone | | | wt/SLE/2014/Makona-J0060, partial genome |
| AJP14328 | Sierra | Oct. 10, 2014 | Mar. 1, 2015 | Zaire ebolavirus isolate Ebolavirus/H.sapiens- |
| | Leone | | | wt/SLE/2014/Makona-J0061, partial genome |
| AJP15137 | Sierra | Oct. 10, 2014 | Mar. 1, 2015 | Zaire ebolavirus isolate Ebolavirus/H.sapiens- |
| | Leone | | | wt/SLE/2014/Makona-J0062, partial genome |
| AJP14337 | Sierra | Oct. 10, 2014 | Mar. 1, 2015 | Zaire ebolavirus isolate Ebolavirus/H.sapiens- |
| | Leone | | | wt/SLE/2014/Makona-J0063, partial genome |
| AJP15146 | Sierra | Oct. 9, 2014 | Mar. 1, 2015 | Zaire ebolavirus isolate Ebolavirus/H.sapiens- |
| | Leone | | | wt/SLE/2014/Makona-J0064, partial genome |
| AJP15155 | Sierra | Oct. 9, 2014 | Mar. 1, 2015 | Zaire ebolavirus isolate Ebolavirus/H.sapiens- |
| | Leone | | | wt/SLE/2014/Makona-J0065, partial genome |
| AJP15164 | Sierra | Oct. 10, 2014 | Mar. 1, 2015 | Zaire ebolavirus isolate Ebolavirus/H.sapiens- |
| | Leone | | | wt/SLE/2014/Makona-J0066, partial genome |
| AJP15173 | Sierra | Oct. 9, 2014 | Mar. 1, 2015 | Zaire ebolavirus isolate Ebolavirus/H.sapiens- |
| | Leone | | | wt/SLE/2014/Makona-J0067, partial genome |
| AJP15182 | Sierra | Oct. 9, 2014 | Mar. 1, 2015 | Zaire ebolavirus isolate Ebolavirus/H.sapiens- |
| | Leone | | | wt/SLE/2014/Makona-J0068, partial genome |
| AJP15191 | Sierra | Oct. 9, 2014 | Mar. 1, 2015 | Zaire ebolavirus isolate Ebolavirus/H.sapiens- |
| | Leone | | | wt/SLE/2014/Makona-J0069, partial genome |
| AJP15209 | Sierra | Oct. 9, 2014 | Mar. 1, 2015 | Zaire ebolavirus isolate Ebolavirus/H.sapiens- |
| | Leone | | | wt/SLE/2014/Makona-J0070, partial genome |
| AJP15218 | Sierra | Oct. 9, 2014 | Mar. 1, 2015 | Zaire ebolavirus isolate Ebolavirus/H.sapiens- |
| | Leone | | | wt/SLE/2014/Makona-J0071, partial genome |
| AJP15227 | Sierra | Oct. 9, 2014 | Mar. 1, 2015 | Zaire ebolavirus isolate Ebolavirus/H.sapiens- |
| | Leone | | | wt/SLE/2014/Makona-J0072, partial genome |
| AJP15236 | Sierra | Oct. 12, 2014 | Mar. 1, 2015 | Zaire ebolavirus isolate Ebolavirus/H.sapiens- |
| | Leone | | | wt/SLE/2014/Makona-J0073, partial genome |
| AJP15245 | Sierra | Oct. 13, 2014 | Mar. 1, 2015 | Zaire ebolavirus isolate Ebolavirus/H.sapiens- |
| | Leone | | | wt/SLE/2014/Makona-J0074, partial genome |
| AJP15281 | Sierra | Oct. 17, 2014 | Mar. 1, 2015 | Zaire ebolavirus isolate Ebolavirus/H.sapiens- |
| | Leone | | | wt/SLE/2014/Makona-J0075, partial genome |
| AJP15290 | Sierra | Oct. 17, 2014 | Mar. 1, 2015 | Zaire ebolavirus isolate Ebolavirus/H.sapiens- |
| | Leone | | | wt/SLE/2014/Makona-J0076, partial genome |
| AJP15299 | Sierra | Oct. 17, 2014 | Mar. 1, 2015 | Zaire ebolavirus isolate Ebolavirus/H.sapiens- |
| | Leone | | | wt/SLE/2014/Makona-J0077, partial genome |
| AJP15308 | Sierra | Oct. 18, 2014 | Mar. 1, 2015 | Zaire ebolavirus isolate Ebolavirus/H.sapiens- |
| | Leone | | | wt/SLE/2014/Makona-J0078, partial genome |
| AJP15326 | Sierra | Oct. 16, 2014 | Mar. 1, 2015 | Zaire ebolavirus isolate Ebolavirus/H.sapiens- |
| | Leone | | | wt/SLE/2014/Makona-J0079, partial genome |
| AJP15335 | Sierra | Oct. 18, 2014 | Mar. 1, 2015 | Zaire ebolavirus isolate Ebolavirus/H.sapiens- |
| | Leone | | | wt/SLE/2014/Makona-J0080, partial genome |
| AJP15344 | Sierra | Oct. 16, 2014 | Mar. 1, 2015 | Zaire ebolavirus isolate Ebolavirus/H.sapiens- |
| | Leone | | | wt/SLE/2014/Makona-J0081, partial genome |
| AJP15353 | Sierra | Oct. 16, 2014 | Mar. 1, 2015 | Zaire ebolavirus isolate Ebolavirus/H.sapiens- |
| | Leone | | | wt/SLE/2014/Makona-J0082, partial genome |
| AJP15362 | Sierra | Oct. 18, 2014 | Mar. 1, 2015 | Zaire ebolavirus isolate Ebolavirus/H.sapiens- |
| | Leone | | | wt/SLE/2014/Makona-J0083, partial genome |
| AJP15371 | Sierra | Oct. 16, 2014 | Mar. 1, 2015 | Zaire ebolavirus isolate Ebolavirus/H.sapiens- |
| | Leone | | | wt/SLE/2014/Makona-J0084, partial genome |
| AJP14400 | Sierra | Oct. 17, 2014 | Mar. 1, 2015 | Zaire ebolavirus isolate Ebolavirus/H.sapiens- |
| | Leone | | | wt/SLE/2014/Makona-J0085, partial genome |
| AJP14417 | Sierra | Oct. 23, 2014 | Mar. 1, 2015 | Zaire ebolavirus isolate Ebolavirus/H.sapiens- |
| | Leone | | | wt/SLE/2014/Makona-J0087, partial genome |
| AJP14426 | Sierra | Oct. 20, 2014 | Mar. 1, 2015 | Zaire ebolavirus isolate Ebolavirus/H.sapiens- |
| | Leone | | | wt/SLE/2014/Makona-J0088, partial genome |
| AJP15416 | Sierra | Oct. 23, 2014 | Mar. 1, 2015 | Zaire ebolavirus isolate Ebolavirus/H.sapiens- |
| | Leone | | | wt/SLE/2014/Makona-J0089, partial genome |
| AJP14435 | Sierra | Oct. 23, 2014 | Mar. 1, 2015 | Zaire ebolavirus isolate Ebolavirus/H.sapiens- |
| | Leone | | | wt/SLE/2014/Makona-J0090, partial genome |
| AJP15425 | Sierra | Oct. 25, 2014 | Mar. 1, 2015 | Zaire ebolavirus isolate Ebolavirus/H.sapiens- |
| | Leone | | | wt/SLE/2014/Makona-J0091, partial genome |
| AJP14444 | Sierra | Oct. 25, 2014 | Mar. 1, 2015 | Zaire ebolavirus isolate Ebolavirus/H.sapiens- |
| | Leone | | | wt/SLE/2014/Makona-J0092, partial genome |
| AJP15434 | Sierra | Oct. 24, 2014 | Mar. 1, 2015 | Zaire ebolavirus isolate Ebolavirus/H.sapiens- |
| | Leone | | | wt/SLE/2014/Makona-J0093, partial genome |
| AJP15443 | Sierra | Oct. 27, 2014 | Mar. 1, 2015 | Zaire ebolavirus isolate Ebolavirus/H.sapiens- |
| | Leone | | | wt/SLE/2014/Makona-J0094, partial genome |
| AJP14471 | Sierra | Oct. 27, 2014 | Mar. 1, 2015 | Zaire ebolavirus isolate Ebolavirus/H.sapiens- |
| | Leone | | | wt/SLE/2014/Makona-J0095, partial genome |
| AJP14480 | Sierra | Oct. 27, 2014 | Mar. 1, 2015 | Zaire ebolavirus isolate Ebolavirus/H.sapiens- |
| | Leone | | | wt/SLE/2014/Makona-J0096, partial genome |
| AJP15460 | Sierra | Oct. 28, 2014 | Mar. 1, 2015 | Zaire ebolavirus isolate Ebolavirus/H.sapiens- |
| | Leone | | | wt/SLE/2014/Makona-J0098, partial genome |
| AJP15469 | Sierra | Oct. 28, 2014 | Mar. 1, 2015 | Zaire ebolavirus isolate Ebolavirus/H.sapiens- |
| | Leone | | | wt/SLE/2014/Makona-J0099, partial genome |
| AJP14489 | Sierra | Oct. 26, 2014 | Mar. 1, 2015 | Zaire ebolavirus isolate Ebolavirus/H.sapiens- |
| | Leone | | | wt/SLE/2014/Makona-J0100, partial genome |
| AJP14498 | Sierra | Oct. 29, 2014 | Mar. 1, 2015 | Zaire ebolavirus isolate Ebolavirus/H.sapiens- |
| | Leone | | | wt/SLE/2014/Makona-J0101, partial genome |
| AJP14507 | Sierra | Oct. 27, 2014 | Mar. 1, 2015 | Zaire ebolavirus isolate Ebolavirus/H.sapiens- |
| | Leone | | | wt/SLE/2014/Makona-J0102, partial genome |
| AJP14516 | Sierra | Oct. 29, 2014 | Mar. 1, 2015 | Zaire ebolavirus isolate Ebolavirus/H.sapiens- |
| | Leone | | | wt/SLE/2014/Makona-J0103, partial genome |
| AJP15478 | Sierra | Oct. 29, 2014 | Mar. 1, 2015 | Zaire ebolavirus isolate Ebolavirus/H.sapiens- |
| | Leone | | | wt/SLE/2014/Makona-J0104, partial genome |
| AJP15487 | Sierra | Oct. 29, 2014 | Mar. 1, 2015 | Zaire ebolavirus isolate Ebolavirus/H.sapiens- |
| | Leone | | | wt/SLE/2014/Makona-J0105, partial genome |
| AJP15496 | Sierra | Oct. 28, 2014 | Mar. 1, 2015 | Zaire ebolavirus isolate Ebolavirus/H.sapiens- |
| | Leone | | | wt/SLE/2014/Makona-J0106, partial genome |
| AJP15505 | Sierra | Oct. 29, 2014 | Mar. 1, 2015 | Zaire ebolavirus isolate Ebolavirus/H.sapiens- |
| | Leone | | | wt/SLE/2014/Makona-J0107, partial genome |
| AJP14525 | Sierra | Oct. 28, 2014 | Mar. 1, 2015 | Zaire ebolavirus isolate Ebolavirus/H.sapiens- |
| | Leone | | | wt/SLE/2014/Makona-J0108, partial genome |
| AJP13941 | Sierra | Oct. 31, 2014 | Mar. 1, 2015 | Zaire ebolavirus isolate Ebolavirus/H.sapiens- |
| | Leone | | | wt/SLE/2014/Makona-J0109, partial genome |
| AJP14534 | Sierra | Oct. 31, 2014 | Mar. 1, 2015 | Zaire ebolavirus isolate Ebolavirus/H.sapiens- |
| | Leone | | | wt/SLE/2014/Makona-J0110, partial genome |
| AJP13950 | Sierra | Oct. 30, 2014 | Mar. 1, 2015 | Zaire ebolavirus isolate Ebolavirus/H.sapiens- |
| | Leone | | | wt/SLE/2014/Makona-J0111, partial genome |
| AJP14543 | Sierra | Oct. 31, 2014 | Mar. 1, 2015 | Zaire ebolavirus isolate Ebolavirus/H.sapiens- |
| | Leone | | | wt/SLE/2014/Makona-J0112, partial genome |
| AJP14552 | Sierra | Oct. 30, 2014 | Mar. 1, 2015 | Zaire ebolavirus isolate Ebolavirus/H.sapiens- |
| | Leone | | | wt/SLE/2014/Makona-J0113, partial genome |
| AJP13959 | Sierra | Oct. 29, 2014 | Mar. 1, 2015 | Zaire ebolavirus isolate Ebolavirus/H.sapiens- |
| | Leone | | | wt/SLE/2014/Makona-J0114, partial genome |
| AJP13968 | Sierra | Oct. 29, 2014 | Mar. 1, 2015 | Zaire ebolavirus isolate Ebolavirus/H.sapiens- |
| | Leone | | | wt/SLE/2014/Makona-J0115, partial genome |
| AJP14570 | Sierra | Oct. 30, 2014 | Mar. 1, 2015 | Zaire ebolavirus isolate Ebolavirus/H.sapiens- |
| | Leone | | | wt/SLE/2014/Makona-J0116, partial genome |
| AJP14579 | Sierra | Oct. 31, 2014 | Mar. 1, 2015 | Zaire ebolavirus isolate Ebolavirus/H.sapiens- |
| | Leone | | | wt/SLE/2014/Makona-J0117, partial genome |
| AJP13977 | Sierra | Oct. 31, 2014 | Mar. 1, 2015 | Zaire ebolavirus isolate Ebolavirus/H.sapiens- |
| | Leone | | | wt/SLE/2014/Makona-J0118, partial genome |
| AJP14597 | Sierra | Oct. 30, 2014 | Mar. 1, 2015 | Zaire ebolavirus isolate Ebolavirus/H.sapiens- |
| | Leone | | | wt/SLE/2014/Makona-J0119, partial genome |
| AJP13986 | Sierra | Oct. 30, 2014 | Mar. 1, 2015 | Zaire ebolavirus isolate Ebolavirus/H.sapiens- |
| | Leone | | | wt/SLE/2014/Makona-J0120, partial genome |
| AJP14615 | Sierra | Oct. 29, 2014 | Mar. 1, 2015 | Zaire ebolavirus isolate Ebolavirus/H.sapiens- |
| | Leone | | | wt/SLE/2014/Makona-J0121, partial genome |
| AJP14633 | Sierra | Oct. 29, 2014 | Mar. 1, 2015 | Zaire ebolavirus isolate Ebolavirus/H.sapiens- |
| | Leone | | | wt/SLE/2014/Makona-J0122, partial genome |
| AJP14642 | Sierra | Nov. 1, 2014 | Mar. 1, 2015 | Zaire ebolavirus isolate Ebolavirus/H.sapiens- |
| | Leone | | | wt/SLE/2014/Makona-J0123, partial genome |
| AJP14651 | Sierra | Nov. 2, 2014 | Mar. 1, 2015 | Zaire ebolavirus isolate Ebolavirus/H.sapiens- |
| | Leone | | | wt/SLE/2014/Makona-J0124, partial genome |
| AJP14660 | Sierra | Oct. 31, 2014 | Mar. 1, 2015 | Zaire ebolavirus isolate Ebolavirus/H.sapiens- |
| | Leone | | | wt/SLE/2014/Makona-J0125, partial genome |
| AJP14669 | Sierra | Oct. 31, 2014 | Mar. 1, 2015 | Zaire ebolavirus isolate Ebolavirus/H.sapiens- |
| | Leone | | | wt/SLE/2014/Makona-J0126, partial genome |
| AJP14678 | Sierra | Oct. 30, 2014 | Mar. 1, 2015 | Zaire ebolavirus isolate Ebolavirus/H.sapiens- |
| | Leone | | | wt/SLE/2014/Makona-J0127, partial genome |
| AJP14687 | Sierra | Oct. 31, 2014 | Mar. 1, 2015 | Zaire ebolavirus isolate Ebolavirus/H.sapiens- |
| | Leone | | | wt/SLE/2014/Makona-J0128, partial genome |
| AJP13995 | Sierra | Nov. 2, 2014 | Mar. 1, 2015 | Zaire ebolavirus isolate Ebolavirus/H.sapiens- |
| | Leone | | | wt/SLE/2014/Makona-J0129, partial genome |
| AJP14705 | Sierra | Nov. 2, 2014 | Mar. 1, 2015 | Zaire ebolavirus isolate Ebolavirus/H.sapiens- |
| | Leone | | | wt/SLE/2014/Makona-J0130, partial genome |
| AJP14714 | Sierra | Oct. 30, 2014 | Mar. 1, 2015 | Zaire ebolavirus isolate Ebolavirus/H.sapiens- |
| | Leone | | | wt/SLE/2014/Makona-J0131, partial genome |
| AJP14004 | Sierra | Nov. 2, 2014 | Mar. 1, 2015 | Zaire ebolavirus isolate Ebolavirus/H.sapiens- |
| | Leone | | | wt/SLE/2014/Makona-J0132, partial genome |
| AJP14723 | Sierra | Oct. 31, 2014 | Mar. 1, 2015 | Zaire ebolavirus isolate Ebolavirus/H.sapiens- |
| | Leone | | | wt/SLE/2014/Makona-J0133, partial genome |
| AJP14732 | Sierra | Oct. 31, 2014 | Mar. 1, 2015 | Zaire ebolavirus isolate Ebolavirus/H.sapiens- |
| | Leone | | | wt/SLE/2014/Makona-J0134, partial genome |
| AJP14013 | Sierra | Nov. 3, 2014 | Mar. 1, 2015 | Zaire ebolavirus isolate Ebolavirus/H.sapiens- |
| | Leone | | | wt/SLE/2014/Makona-J0135, partial genome |
| AJP14750 | Sierra | Nov. 1, 2014 | Mar. 1, 2015 | Zaire ebolavirus isolate Ebolavirus/H.sapiens- |
| | Leone | | | wt/SLE/2014/Makona-J0136, partial genome |
| AJP14759 | Sierra | Nov. 1, 2014 | Mar. 1, 2015 | Zaire ebolavirus isolate Ebolavirus/H.sapiens- |
| | Leone | | | wt/SLE/2014/Makona-J0137, partial genome |
| AJP14768 | Sierra | Nov. 1, 2014 | Mar. 1, 2015 | Zaire ebolavirus isolate Ebolavirus/H.sapiens- |
| | Leone | | | wt/SLE/2014/Makona-J0138, partial genome |
| AJP14777 | Sierra | Oct. 30, 2014 | Mar. 1, 2015 | Zaire ebolavirus isolate Ebolavirus/H.sapiens- |
| | Leone | | | wt/SLE/2014/Makona-J0139, partial genome |
| AJP14022 | Sierra | Oct. 30, 2014 | Mar. 1, 2015 | Zaire ebolavirus isolate Ebolavirus/H.sapiens- |
| | Leone | | | wt/SLE/2014/Makona-J0140, partial genome |
| AJP14795 | Sierra | Nov. 4, 2014 | Mar. 1, 2015 | Zaire ebolavirus isolate Ebolavirus/H.sapiens- |
| | Leone | | | wt/SLE/2014/Makona-J0141, partial genome |
| AJP14804 | Sierra | Nov. 3, 2014 | Mar. 1, 2015 | Zaire ebolavirus isolate Ebolavirus/H.sapiens- |
| | Leone | | | wt/SLE/2014/Makona-J0142, partial genome |
| AJP14031 | Sierra | Nov. 4, 2014 | Mar. 1, 2015 | Zaire ebolavirus isolate Ebolavirus/H.sapiens- |
| | Leone | | | wt/SLE/2014/Makona-J0143, partial genome |
| AJP14040 | Sierra | Nov. 4, 2014 | Mar. 1, 2015 | Zaire ebolavirus isolate Ebolavirus/H.sapiens- |
| | Leone | | | wt/SLE/2014/Makona-J0144, partial genome |
| AJP14831 | Sierra | Nov. 6, 2014 | Mar. 1, 2015 | Zaire ebolavirus isolate Ebolavirus/H.sapiens- |
| | Leone | | | wt/SLE/2014/Makona-J0145, partial genome |
| AJP14085 | Sierra | Nov. 7, 2014 | Mar. 1, 2015 | Zaire ebolavirus isolate Ebolavirus/H.sapiens- |
| | Leone | | | wt/SLE/2014/Makona-J0146, partial genome |
| AJP14094 | Sierra | Nov. 7, 2014 | Mar. 1, 2015 | Zaire ebolavirus isolate Ebolavirus/H.sapiens- |
| | Leone | | | wt/SLE/2014/Makona-J0147, partial genome |
| AJP14103 | Sierra | Nov. 6, 2014 | Mar. 1, 2015 | Zaire ebolavirus isolate Ebolavirus/H.sapiens- |
| | Leone | | | wt/SLE/2014/Makona-J0148, partial genome |
| AJP14849 | Sierra | Nov. 7, 2014 | Mar. 1, 2015 | Zaire ebolavirus isolate Ebolavirus/H.sapiens- |
| | Leone | | | wt/SLE/2014/Makona-J0149, partial genome |
| AJP14858 | Sierra | Nov. 6, 2014 | Mar. 1, 2015 | Zaire ebolavirus isolate Ebolavirus/H.sapiens- |
| | Leone | | | wt/SLE/2014/Makona-J0150, partial genome |
| AJP14112 | Sierra | Nov. 7, 2014 | Mar. 1, 2015 | Zaire ebolavirus isolate Ebolavirus/H.sapiens- |
| | Leone | | | wt/SLE/2014/Makona-J0151, partial genome |
| AJP14121 | Sierra | Nov. 7, 2014 | Mar. 1, 2015 | Zaire ebolavirus isolate Ebolavirus/H.sapiens- |
| | Leone | | | wt/SLE/2014/Makona-J0152, partial genome |
| AJP14139 | Sierra | Nov. 7, 2014 | Mar. 1, 2015 | Zaire ebolavirus isolate Ebolavirus/H.sapiens- |
| | Leone | | | wt/SLE/2014/Makona-J0153, partial genome |
| AJP14148 | Sierra | Nov. 7, 2014 | Mar. 1, 2015 | Zaire ebolavirus isolate Ebolavirus/H.sapiens- |
| | Leone | | | wt/SLE/2014/Makona-J0154, partial genome |
| AJP14867 | Sierra | Nov. 7, 2014 | Mar. 1, 2015 | Zaire ebolavirus isolate Ebolavirus/H.sapiens- |
| | Leone | | | wt/SLE/2014/Makona-J0155, partial genome |
| AJP14876 | Sierra | Nov. 7, 2014 | Mar. 1, 2015 | Zaire ebolavirus isolate Ebolavirus/H.sapiens- |
| | Leone | | | wt/SLE/2014/Makona-J0156, partial genome |
| AJP14885 | Sierra | Nov. 6, 2014 | Mar. 1, 2015 | Zaire ebolavirus isolate Ebolavirus/H.sapiens- |
| | Leone | | | wt/SLE/2014/Makona-J0157, partial genome |
| AJP14894 | Sierra | Nov. 8, 2014 | Mar. 1, 2015 | Zaire ebolavirus isolate Ebolavirus/H.sapiens- |
| | Leone | | | wt/SLE/2014/Makona-J0158, partial genome |
| AJP14166 | Sierra | Nov. 5, 2014 | Mar. 1, 2015 | Zaire ebolavirus isolate Ebolavirus/H.sapiens- |
| | Leone | | | wt/SLE/2014/Makona-J0159, partial genome |
| AJP14184 | Sierra | Nov. 7, 2014 | Mar. 1, 2015 | Zaire ebolavirus isolate Ebolavirus/H.sapiens- |
| | Leone | | | wt/SLE/2014/Makona-J0160, partial genome |
| AJP14903 | Sierra | Nov. 8, 2014 | Mar. 1, 2015 | Zaire ebolavirus isolate Ebolavirus/H.sapiens- |
| | Leone | | | wt/SLE/2014/Makona-J0161, partial genome |
| AJP14193 | Sierra | Nov. 9, 2014 | Mar. 1, 2015 | Zaire ebolavirus isolate Ebolavirus/H.sapiens- |
| | Leone | | | wt/SLE/2014/Makona-J0162, partial genome |
| AJP14912 | Sierra | Nov. 8, 2014 | Mar. 1, 2015 | Zaire ebolavirus isolate Ebolavirus/H.sapiens- |
| | Leone | | | wt/SLE/2014/Makona-J0163, partial genome |
| AJP14202 | Sierra | Nov. 9, 2014 | Mar. 1, 2015 | Zaire ebolavirus isolate Ebolavirus/H.sapiens- |
| | Leone | | | wt/SLE/2014/Makona-J0164, partial genome |
| AJP14921 | Sierra | Nov. 8, 2014 | Mar. 1, 2015 | Zaire ebolavirus isolate Ebolavirus/H.sapiens- |
| | Leone | | | wt/SLE/2014/Makona-J0165, partial genome |
| AJP14930 | Sierra | Nov. 8, 2014 | Mar. 1, 2015 | Zaire ebolavirus isolate Ebolavirus/H.sapiens- |
| | Leone | | | wt/SLE/2014/Makona-J0166, partial genome |
| AJP14211 | Sierra | Nov. 8, 2014 | Mar. 1, 2015 | Zaire ebolavirus isolate Ebolavirus/H.sapiens- |
| | Leone | | | wt/SLE/2014/Makona-J0167, partial genome |
| AJP14939 | Sierra | Nov. 11, 2014 | Mar. 1, 2015 | Zaire ebolavirus isolate Ebolavirus/H.sapiens- |
| | Leone | | | wt/SLE/2014/Makona-J0168, partial genome |
| AJP14948 | Sierra | Nov. 9, 2014 | Mar. 1, 2015 | Zaire ebolavirus isolate Ebolavirus/H.sapiens- |
| | Leone | | | wt/SLE/2014/Makona-J0169, partial genome |
| AJP14220 | Sierra | Nov. 10, 2014 | Mar. 1, 2015 | Zaire ebolavirus isolate Ebolavirus/H.sapiens- |
| | Leone | | | wt/SLE/2014/Makona-J0170, partial genome |
| AJP14957 | Sierra | Nov. 10, 2014 | Mar. 1, 2015 | Zaire ebolavirus isolate Ebolavirus/H.sapiens- |
| | Leone | | | wt/SLE/2014/Makona-J0171, partial genome |
| AJP14229 | Sierra | Nov. 11, 2014 | Mar. 1, 2015 | Zaire ebolavirus isolate Ebolavirus/H.sapiens- |
| | Leone | | | wt/SLE/2014/Makona-J0172, partial genome |
| AJP14238 | Sierra | Nov. 11, 2014 | Mar. 1, 2015 | Zaire ebolavirus isolate Ebolavirus/H.sapiens- |
| | Leone | | | wt/SLE/2014/Makona-J0173, partial genome |
| AJP14966 | Sierra | Nov. 11, 2014 | Mar. 1, 2015 | Zaire ebolavirus isolate Ebolavirus/H.sapiens- |
| | Leone | | | wt/SLE/2014/Makona-J0174, partial genome |
| AJP14975 | Sierra | Nov. 11, 2014 | Mar. 1, 2015 | Zaire ebolavirus isolate Ebolavirus/H.sapiens- |
| | Leone | | | wt/SLE/2014/Makona-J0175, partial genome |
| AIW47453 | Guinea | 2014 March | Nov. 10, 2014 | Zaire ebolavirus isolate H.sapiens- |
| | | | | tc/GIN/14/WPG-C05, complete genome |
| AIW47461 | Guinea | 2014 March | Nov. 10, 2014 | Zaire ebolavirus isolate H.sapiens- |
| | | | | tc/GIN/14/WPG-C07, complete genome |
| AIW47469 | Guinea | 2014 March | Nov. 10, 2014 | Zaire ebolavirus isolate H.sapiens- |
| | | | | tc/GIN/14/WPG-C15, complete genome |
| ALH21455 | Guinea | 2014 | Oct. 10, 2015 | Zaire ebolavirus isolate H.sapiens- |
| | | | | wt/GIN/2014/Makona-Conakry-CREMS-1022, |
| | | | | complete genome |
| ALH21464 | Guinea | 2014 | Oct. 10, 2015 | Zaire ebolavirus isolate H.sapiens- |
| | | | | wt/GIN/2014/Makona-Conakry-CREMS-2214, |
| | | | | complete genome |
| AHX24668 | Guinea | 2014 | Apr. 18, 2014 | Zaire ebolavirus isolate H.sapiens- |
| | | | | wt/GIN/2014/Makona-Gueckedou-C05, complete |
| | | | | genome |
| AHX24659 | Guinea | 2014 | Apr. 18, 2014 | Zaire ebolavirus isolate H.sapiens- |
| | | | | wt/GIN/2014/Makona-Gueckedou-C07, complete |
| | | | | genome |
| AHX24650 | Guinea | 2014 | Apr. 18, 2014 | Zaire ebolavirus isolate H.sapiens- |
| | | | | wt/GIN/2014/Makona-Kissidougou-C15, |
| | | | | complete genome |
| AJT59735 | Democratic | 2003 | Mar. 17, 2015 | Zaire ebolavirus isolate Kelle 1 NP protein (NP), |
| | Republic of | | | VP35 protein (VP35), VP40 protein (VP40), GP |
| | the Congo | | | protein (GP), VP30 protein (VP30), VP24 protein |
| | | | | (VP24), and L protein (L) genes, complete cds |
| AER59717 | Democratic | Aug. 31, 2007 | Nov. 7, 2011 | Zaire ebolavirus isolate M-M, partial genome |
| | Republic of | | | |
| | the Congo | | | |
| AKI84266 | Democratic | 1995 | Jun. 2, 2015 | Zaire ebolavirus isolate Zaire ebolavirus H. |
| | Republic of | | | sapiens-tc/ZAI/1995/Zaire-199510621, partial |
| | the Congo | | | genome |
| AKI84257 | Sierra | 2014 | Jun. 2, 2015 | Zaire ebolavirus isolate Zaire |
| | Leone | | | ebolavirus/H.sapiens-tc/SL/2014/Makona- |
| | | | | SL3864.1, partial genome |

TABLE 10
Marburg Amino Acid Sequences

| SEQ | |
|---|---|
| ID | |
| NO. | Description |
| 57 | Marburg_GP12_Musoke1980_HuIgGk |
| 58 | Marburg_GP12_Musoke1980_HuIgGk without signal sequence |
| 59 | Marburg_GP12_Ravn1987_HuIgGk |
| 60 | Marburg_GP12_Ravn1987_HuIgGk without signal sequence |
| 61 | Marburg_GP12_Uganda2007_HuIgGk |
| 62 | Marburg_GP12_Uganda2007_HuIgGk without signal sequence |
| 63 | Musoke\|NA |
| 64 | M/S.Africa/Johannesburg/1975/Ozolin\|NA |
| 65 | pp3_guinea_pig_lethal_variant\|NA |
| 66 | pp4_guinea_pig_nonlethal_variant\|NA |
| 67 | Popp\|NA |
| 68 | Musoke\|NA_1 |
| 69 | M/S.Africa/Johannesburg/1975/Ozolin\|NA_1 |
| 70 | M/Germany/Marburg/1967/Ratayczak\|NA |
| 71 | M/Kenya/Kitum_Cave/1987/Ravn\|NA |
| 72 | Popp\|NA_1 |
| 73 | Marburg_virus/H.sapiens_tc/KEN/1980/Mt._Elgon_Musoke\|NA |
| 74 | Germany_{——}Marburg\|1967 |
| 75 | Ci67\|1967 |
| 76 | Ci67\|1967_1 |
| 77 | Ci67\|1967_2 |
| 78 | Kenya\|1987 |
| 79 | Ravn\|1987 |
| 80 | R2\|1987 |
| 81 | R3\|1987 |
| 82 | R1\|1987 |
| 83 | Ravn_virus/H.sapiens_tc/KEN/1987/Kitum_Cave_810040\|1987 |
| 84 | Ravn_virus/H._sapiens_tc/KEN/1987/Ravn_CDC811103\|08/19/ |
| | 1987 |
| 85 | MARV/H.sapiens_tc/COD/1999/03_DRC\|1999 |
| 86 | MARV/H.sapiens_tc/COD/1999/01_DRC\|1999 |
| 87 | MARV/H.sapiens_tc/COD/1999/06_DRC\|1999 |
| 88 | MARV/H.sapiens_tc/COD/1999/02_DRC\|1999 |
| 89 | MARV/H.sapiens_tc/COD/1999/03_DRC\|1999 |
| 90 | MARV/H.sapiens_tc/COD/1999/05_DRC\|1999 |
| 91 | MARV/H.sapiens_tc/COD/2000/14_DRC\|2000 |
| 92 | 09DRC99\|05/1999 |
| 93 | MARV/H.sapiens_tc/COD/2000/25_DRC\|2000 |
| 94 | MARV/H.sapiens_tc/COD/2000/28_DRC\|2000 |
| 95 | 05DRC99\|05/1999 |
| 96 | MARV/H.sapiens_tc/COD/2000/19_DRC\|2000 |
| 97 | 07DRC99\|05/1999 |
| 98 | MARV/H.sapiens_tc/COD/2000/18_DRC\|2000 |
| 99 | MARV/H.sapiens_tc/COD/2000/17_DRC\|2000 |
| 100 | MARV/H.sapiens_tc/COD/2000/16_DRC\|2000 |
| 101 | MARV/H.sapiens_tc/COD/2000/29_DRC\|2000 |
| 102 | MARV/H.sapiens_tc/COD/2000/30_DRC\|2000 |
| 103 | MARV/H.sapiens_tc/COD/2000/32_DRC\|2000 |
| 104 | MARV/H.sapiens_tc/COD/2000/21_DRC\|2000 |
| 105 | MARV/H.sapiens_tc/COD/2000/20_DRC\|2000 |
| 106 | MARV/H.sapiens_tc/COD/2000/24_DRC\|2000 |
| 107 | MARV/H.sapiens_tc/COD/2000/13_DRC\|2000 |
| 108 | MARV/H.sapiens_tc/COD/2000/15_DRC\|2000 |
| 109 | MARV/H.sapiens_tc/COD/2000/26_DRC\|2000 |
| 110 | MARV/H.sapiens_tc/COD/2000/34_DRC\|2000 |
| 111 | MARV/H.sapiens_tc/COD/2000/22_DRC\|2000 |
| 112 | MARV/H.sapiens_tc/COD/2000/33_DRC\|2000 |
| 113 | MARV/H.sapiens_tc/COD/2000/27_DRC\|2000 |
| 114 | MARV/H.sapiens_tc/COD/2000/23_DRC\|2000 |
| 115 | Ang1386\|03/2005 |
| 116 | Ang1381\|03/2005 |
| 117 | Marburg_virus/H.sapiens_tc/AGO/2005/Angola_200501379\|03/ |
| | 13/2005 |
| 118 | Marburg_virus/H.sapiens_tc/AGO/2005/Angola_810820\|03/13/ |
| | 2005 |
| 119 | Ang0215\|04/2005 |
| 120 | Ang0214\|04/2005 |
| 121 | Ang0126\|04/2005 |
| 122 | Ang0754\|04/2005 |
| 123 | Ang0998\|05/2005 |
| 124 | Uganda_02Uga07\|2007 |
| 125 | Uganda_01Uga07\|2007 |
| 126 | MARV/R.aegyptiacus_tc/UGA/2008/164_Qbat\|2008 |
| 127 | MARV/R.aegyptiacus_tc/UGA/2008/53_Qbat\|2008 |
| 128 | Leiden\|2008 |
| 129 | RAVV/R.aegyptiacus_tc/UGA/2009/1304_Qbat\|2009 |
| 130 | MARV/R.aegyptiacus_tc/UGA/2009/1328_Qbat\|2009 |
| 131 | MARV/R.aegyptiacus_tc/UGA/2009/843_Qbat\|2009 |
| 132 | MARV/R.aegyptiacus_tc/UGA/2009/1175_Qbat\|2009 |
| 133 | MARV/R.aegyptiacus_tc/UGA/2009/914_Qbat\|2009 |
| 134 | NML/M.musculus_lab/AGO/2005/Ang_MA_P2\|01/15/2014 |

TABLE 11
Marburg DNA Sequences

| SEQ | |
|---|---|
| ID | |
| NO. | Description |
| 135 | Marburg_GP12_Musoke1980_HuIgGk - ORF |
| 136 | Marburg_GP12_Ravn1987_HuIgGk - ORF |
| 137 | Marburg_GP12_Uganda2007_HuIgGk - ORF |
| 166 | From 5′ end to 3′ end: 5′ UTR SEQ ID NO: 142, DNA ORF SEQ |
| | ID NO: 135, and 3′ UTR SEQ ID NO: 143 |
| 167 | From 5′ end to 3′ end: 5′ UTR SEQ ID NO: 144, DNA ORF SEQ |
| | ID NO: 135, and 3′ UTR SEQ ID NO: 143 |
| 168 | From 5′ end to 3′ end: 5′ UTR SEQ ID NO: 142, DNA ORF SEQ |
| | ID NO: 136, and 3′ UTR SEQ ID NO: 143 |
| 169 | From 5′ end to 3′ end: 5′ UTR SEQ ID NO: 144, DNA ORF SEQ |
| | ID NO: 136, and 3′ UTR SEQ ID NO: 143 |
| 170 | From 5′ end to 3′ end: 5′ UTR SEQ ID NO: 142, DNA ORF SEQ |
| | ID NO: 137, and 3′ UTR SEQ ID NO: 143 |
| 171 | From 5′ end to 3′ end: 5′ UTR SEQ ID NO: 144, DNA ORF SEQ |
| | ID NO: 137, and 3′ UTR SEQ ID NO: 143 |

TABLE 12
Marburg RNA Sequences

| SEQ | |
|---|---|
| ID | |
| NO. | Description |
| 138 | Marburg_GP12_Musoke1980_HuIgGk - ORF |
| 139 | Marburg_GP12_Ravn1987_HuIgGk - ORF |
| 140 | Marburg_GP12_Uganda2007_HuIgGk - ORF |
| 172 | From 5′ end to 3′ end: 5′ UTR SEQ ID NO: 163, RNA ORF SEQ |
| | ID NO: 138, and 3′ UTR SEQ ID NO: 164 |
| 173 | From 5′ end to 3′ end: 5′ UTR SEQ ID NO: 165, RNA ORF SEQ |
| | ID NO: 138, and 3′ UTR SEQ ID NO: 164 |
| 174 | From 5′ end to 3′ end: 5′ UTR SEQ ID NO: 163, RNA ORF SEQ |
| | ID NO: 139, and 3′ UTR SEQ ID NO: 164 |
| 175 | From 5′ end to 3′ end: 5′ UTR SEQ ID NO: 165, RNA ORF SEQ |
| | ID NO: 139, and 3′ UTR SEQ ID NO: 164 |
| 176 | From 5′ end to 3′ end: 5′ UTR SEQ ID NO: 163, RNA ORF SEQ |
| | ID NO: 140, and 3′ UTR SEQ ID NO: 164 |
| 177 | From 5′ end to 3′ end: 5′ UTR SEQ ID NO: 165, RNA ORF SEQ |
| | ID NO: 140, and 3′ UTR SEQ ID NO: 164 |

### EQUIVALENTS

Those skilled in the art will recognize, or be able to ascertain using no more than routine experimentation, many equivalents to the specific embodiments of the disclosure described herein. Such equivalents are intended to be encompassed by the following claims.

All references, including patent documents, disclosed herein are incorporated by reference in their entirety.

1.-2. (canceled)

3. An Ebola virus (EBOV) vaccine, comprising:
at least one ribonucleic acid (RNA) polynucleotide having an open reading frame encoding at least one EBOV antigenic polypeptide or an immunogenic fragment thereof.

4. The vaccine of claim 3, wherein the at least one antigenic polypeptide is selected from EBOV glycoprotein (GP), surface EBOV GP, wild type EBOV GP, mature EBOV GP, secreted wild type EBOV GP, secreted mature EBOV GP, sGP, delta peptide (Δ-peptide), GP1, GP1,2Δ, nucleoprotein NP, viral polymerase L, the polymerase cofactor VP35, the transcriptional activator VP30, VP24, and the matrix protein VP40.

5. The vaccine of claim 3, wherein the vaccine comprises at least one RNA polynucleotide having an open reading frame encoding at least two antigenic polypeptides or immunogenic fragments thereof selected from EBOV glycoprotein (GP), surface EBOV GP, wild type EBOV GP, mature EBOV GP, secreted wild type EBOV GP, secreted mature EBOV GP, sGP, delta peptide (Δ-peptide), GP1, GP1,2Δ, nucleoprotein NP, viral polymerase L, the polymerase cofactor VP35, the transcriptional activator VP30, VP24, and the matrix protein VP40.

6.-8. (canceled)

9. The vaccine of claim 4, wherein the vaccine comprises at least two RNA polynucleotides, each having an open reading frame encoding at least one antigenic polypeptide or an immunogenic fragment thereof selected from EBOV glycoprotein (GP), surface EBOV GP, wild type EBOV GP, sGP, delta peptide (Δ-peptide), GP1, and GP1,2A, wherein the EBOV antigenic polypeptide encoded by one of the open reading frames differs from the EBOV antigenic polypeptide encoded by another of the open reading frames.

10. The vaccine of claim 9, wherein the at least one antigenic polypeptide comprises an amino acid sequence identified by any one of SEQ ID NO: 9-16, 26, 46, 47, 50, 51, 54, 55.

11. The vaccine of claim 10, wherein the at least one RNA polynucleotide is encoded by a nucleic acid sequence identified by any one of SEQ ID NO: 1-8, 17-24, 27, 36, 37, 45, 49, 53, and/or wherein the at least one RNA polynucleotide comprises a nucleic acid sequence identified by any one of SEQ ID NO: 28-44, 48, 52, 56.

12. The vaccine of claim 10, wherein the at least one antigenic polypeptide has an amino acid sequence that has at least 95% identity to an amino acid sequence identified by any one of SEQ ID NO: 9-16, 26, 46, 47, 50, 51, 54, 55.

13. (canceled)

14. The vaccine of claim 10, wherein the at least one antigenic polypeptide has an amino acid sequence that has at least 90% identity to an amino acid sequence of SEQ ID NO: 9-16, 26, 46, 47, 50, 51, 54, 55 and wherein the antigenic polypeptide or immunogenic fragment thereof has membrane fusion activity, attaches to cell receptors, causes fusion of viral and cellular membranes, and/or is responsible for binding of the virus to a cell being infected.

15.-43. (canceled)

44. The vaccine of claim 3, wherein the at least one RNA polynucleotide comprises at least one chemical modification.

45. The vaccine of claim 44, wherein the chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

46. The vaccine of claim 45, wherein the chemical modification is in the 5-position of the uracil.

47. The vaccine of claim 46, wherein the chemical modification is a N1-methylpseudouridine or N1-ethylpseudouridine.

48. The vaccine of claim 46, wherein at least 80% of the uracil in the open reading frame have a chemical modification.

49. The vaccine of claim 46, wherein at least 90% of the uracil in the open reading frame have a chemical modification.

50. The vaccine of claim 46, wherein 100% of the uracil in the open reading frame have a chemical modification.

51. The vaccine of claim 3, wherein at least one RNA polynucleotide further encodes at least one 5′ terminal cap.

52. The vaccine of claim 51, wherein the 5′ terminal cap is 7mG(5′)ppp(5′)NlmpNp.

53. The vaccine of claim 3, wherein at least one antigenic polypeptide or immunogenic fragment thereof is fused to a signal peptide selected from: a HuIgGk signal peptide (METPAQLLFLLLLWLPDTTG; SEQ ID NO: 178); IgE heavy chain epsilon-1 signal peptide (MDWTWILFLVAAATRVHS; SEQ ID NO: 179); Japanese encephalitis PRM signal sequence (MLGSNSGQRVVFTILLLLVAPAYS; SEQ ID NO: 180), VSVg protein signal sequence (MKCLLYLAFLFIGVNCA; SEQ ID NO: 181) and Japanese encephalitis JEV signal sequence (MWLVSLAIVTACAGA; SEQ ID NO: 182).

54. The vaccine of claim 53, wherein the signal peptide is fused to the N-terminus of at least one antigenic polypeptide.

55. The vaccine of claim 53, wherein the signal peptide is fused to the C-terminus of at least one antigenic polypeptide.

56. The vaccine of claim 3, wherein the antigenic polypeptide or immunogenic fragment thereof comprises a mutated N-linked glycosylation site.

57. The vaccine of claim 3 formulated in a nanoparticle.

58. The vaccine of claim 57, wherein the nanoparticle is a lipid nanoparticle.

59. The vaccine of claim 58, wherein the nanoparticle has a mean diameter of 50-200 nm.

60. The vaccine of claim 58, wherein the lipid nanoparticle comprises a cationic lipid, a PEG-modified lipid, a sterol and a non-cationic lipid.

61. The vaccine of claim 60, wherein the lipid nanoparticle carrier comprises a molar ratio of about 20-60% cationic lipid, 0.5-15% PEG-modified lipid, 25-55% sterol, and 25% non-cationic lipid.

62. The vaccine of claim 61, wherein the cationic lipid is an ionizable cationic lipid and the non-cationic lipid is a neutral lipid, and the sterol is a cholesterol.

63. The vaccine of claim 61, wherein the cationic lipid is selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

64. The vaccine of claim 1, wherein the lipid nanoparticle comprises a compound of Formula (I), optionally Compound 3, 18, 20, 25, 26, 29, 30, 60, 108-112, or 122.

65.-67. (canceled)

68. The vaccine of claim 1, further comprising an adjuvant.

69.-73. (canceled)

74. A method of inducing an immune response in a subject, the method comprising administering to the subject the vaccine of claim 3 in an amount effective to produce an antigen-specific immune response in the subject.

75.-99. (canceled)

100. An engineered nucleic acid encoding at least one RNA polynucleotide of a vaccine of claim 3.

101. A pharmaceutical composition for use in vaccination of a subject comprising an effective dose of mRNA encoding an Ebola virus antigen, wherein the effective dose is sufficient to produce detectable levels of antigen as measured in serum of the subject at 1-72 hours post administration.

102. A pharmaceutical composition for use in vaccination of a subject comprising an effective dose of mRNA encoding an Ebola virus antigen, wherein the effective dose is sufficient to produce a 1,000-10,000 neutralization titer produced by neutralizing antibody against said antigen as measured in serum of the subject at 1-72 hours post administration.

103. A vaccine comprising an mRNA encoding an Ebola virus antigen formulated in a lipid nanoparticle comprising compounds of Formula (I): 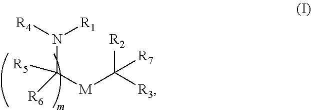
or a salt or isomer thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

104.-130. (canceled)
